(19) United States
(12) Patent Application Publication
(10) Pub. No.: US 2025/0359575 A1
(43) Pub. Date: Nov. 27, 2025

(54) METHOD FOR IMPROVING FLAVOR OF FOOD

(71) Applicant: Ajinomoto Co., Inc., Tokyo (JP)

(72) Inventors: Motonaka KURODA, Kanagawa (JP); Masaki MIZUNO, Kanagawa (JP); Kuniaki KATAYAMA, Kanagawa (JP); Morito SHIMOMURA, Kanagawa (JP); Sachise KARAKAWA, Kanagawa (JP)

(73) Assignee: Ajinomoto Co., Inc., Tokyo (JP)

(21) Appl. No.: 19/288,539

(22) Filed: Aug. 1, 2025

Related U.S. Application Data
(63) Continuation of application No. PCT/JP2024/003375, filed on Feb. 1, 2024.

(30) Foreign Application Priority Data
Feb. 1, 2023 (JP) 2023-013898

(51) Int. Cl. A23L 27/24; A23L 27/00; A23L 27/10; A23L 27/22

(52) U.S. Cl. CPC A23L 27/24; A23L 27/10; A23L 27/22; A23L 27/88; C12Y 302/01017

(57) ABSTRACT

Provided is a technology for improving the flavor of food products. The flavor of food products is improved by the following ingredients (A): (A) Lysozyme-treated product of cell walls of gram-positive bacterium.

### CROSS REFERENCE TO RELATED APPLICATIONS

This application is a Continuation of PCT International Application No. PCT/JP2024/003375, filed on Feb. 1, 2024, which is claiming priority of Japanese Patent Application No. 2023-013898, filed on Feb. 1, 2023, the entire contents of which are incorporated herein by reference.

### TECHNICAL FIELD

The invention relates to techniques for improving the flavor of food products.

### BACKGROUND ARTS

Various techniques to improve food flavor have been reported.

Patent document 1 discloses taste-enhancing flavor bases obtained by fermentation of bacterium such as Corynebacterium glutamicum, Corynebacterium ammoniagenes, Corynebacterium casei, Corynebacterium efficiens, Brevibacterium lactofermentum, Bacillus subtilis and the like.

Patent document 2 discloses a flavor enhancer containing yeast cells after removal of the yeast contents, with examples such as pungency as a flavor.

Patent document 3 discloses a yeast extract containing peptides and nucleic acid-based flavor components that can impart or enhance the kokumi of food products by simultaneously imparting a persistent sense of umami and richness.

Patent document 4 discloses an agent for improving the flavor of food products containing yeast-derived substances such as yeast extracts, with examples such as spice flavor as a flavor.

Patent document 5 discloses a spice sensation enhancer containing a product obtained by a heat reaction between sugar and amino acids in the presence of alcohol.

Patent document 6 discloses a fermented seasoning composition containing a fermented product by lactic acid bacterium and yeast, which can be used for enhancing the stimulating effect of spices, etc.

### PRIOR ART DOCUMENTS

### Patent Documents

Patent document 1: Japanese Patent Laid-open No. 2012-521205
Patent document 2: Japanese Patent Laid-open No. 2018-050562
Patent document 3: Japanese Patent Laid-open No. 2007-049988
Patent document 4: Japanese Patent Laid-open No. 2010-166886
Patent document 5: Japanese Patent Laid-open No. 2016-158507
Patent document 6: WO2017-014253.

### SUMMARY OF INVENTION

### Problem to be Solved by the Invention

The objective of the present invention is to provide a technique for improving the flavor of food products.

### Means to Solve the Problem

The inventors found that the flavor of food products can be significantly improved by blending lysozyme-treated cell walls of gram-positive bacterium (e.g. gram-positive bacterial cells) and completed the present invention.

The present invention can be exemplified as follows.
[1]

A composition for improving a flavor of food product, comprising the following component (A):
(A) lysozyme-treated product of cell walls of a gram-positive bacterium.
[2]

The composition described above (specifically according to [1]), wherein the improvement of flavor is an enhancement of spiciness and/or an impartment of kokumi.
[3]

The composition described above (specifically according to [1] or [2]), wherein the improvement of flavor is an enhancement of pungency of spices and/or an impartment of thickness.
[4]

The composition described above (specifically according to any of [1] to [3]), further comprising a spice.
[5]

A composition, comprising the following component (A) and a spice:
(A) lysozyme-treated product of cell walls of a gram-positive bacterium.
[6]

The composition described above (specifically according to any of [1] to [5]) wherein the component (A) is a lysozyme-treated product of cells of the gram-positive bacterium or fragments thereof.
[7]

The composition described above (specifically according to any of [1] to [6]), wherein the gram-positive bacterium is a bacterium belonging to the phylum Actinobacteria or Firmicutes.
[8]

The composition described above (specifically according to any of [1] to [7]), wherein the gram-positive bacterium is a bacterium belonging to the phylum Actinobacteria.
[9]

The composition described above (specifically according to any of [1] to [8]), wherein the gram-positive bacterium is a coryneform bacterium, a bacterium belonging to the family Bifidobacteriumceae, a bacterium belonging to the family Dermabacteraceae, a bacterium belonging to the family Bacillaceae, a bacterium belonging to the family Enterococcaceae, or a bacterium belonging to the family Lactobacilluseae.
[10]

The composition described above (specifically according to any of [1] to [9]), wherein the gram-positive bacterium is a bacterium of the genus Corynebacterium, a bacterium of the genus Brevibacterium, a bacterium of the genus Bifidobacterium, a bacterium of the genus Brachybacterium, a bacterium of the genus Bacillus, a bacterium of the genus Enterococcus, or a bacterium of the genus Lactobacillus.
[11]

The composition described above (specifically according to any of [1] to [10]), wherein the gram-positive bacterium is Corynebacterium casei, Corynebacterium flavescens, Corynebacterium ammoniagenes (Corynebacterium stationis), Corynebacterium glutamicum, Brevibacterium casei, Bifidobacterium longum, Brachybacterium alimentarium, Bacillus subtilis, Enterococcus faecalis, Lactobacillus mali, Lactobacillus hilgardii or Lactobacillus brevis.
[12]

The composition described above (specifically according to any of [1] to [11]), wherein a content of the component (A) is 0.1% (w/w) or more in terms of dry weight of original cells of the gram-positive bacterium.
[13]

The composition described above (specifically according to any of [1] to [12]), wherein a fraction of the component (A), which has a fractional molecular weight of 10000 Da or less, contains bound diaminopimeric acid.
[14]

The composition described above (specifically according to [13]), wherein a content of the bound diaminopimeric acid in the fraction of the component (A), which has the fractional molecular weight of 10000 Da or less, per 100 g dry weight of the composition, excluding the dry weight of an excipient if the composition contains the excipient, is 20.0 mg or more.
[15]

The composition described above (specifically according to any of [1] to [14]), further comprising the following component (B):
(B) one or more components selected from the group consisting of an L-amino acid, a nucleic acid and an organic acid.
[16]

The composition described above (specifically according to [15]), at least comprising the L-amino acid, wherein the L-amino acid is L-glutamic acid.
[17]

The composition described above (specifically according to [16]), wherein a content of the L-glutamic acid is from 0.1 to 20 parts by weight of the component (A) in terms of dry weight of original cells of the gram-positive bacterium.
[18]

The composition described above (specifically according to any of [4] to [17]), wherein a content of the spice is from 0.2 to 500 parts by weight of the component (A) in terms of dry weight of the original cells of the gram-positive bacterium.
[19]

The composition described above (specifically according to any of [4] to [18]), wherein the spice is one or more spices selected from the group consisting of a spice of the family Lauraceae, a spice of the family Piperaceae, a spice of the family Lamiaceae, a spice of the family Apiaceae, a spice of the family Solanaceae, a spice of the family Myristicaceae, a spice of the family Alliaceae, a spice of the family Myrtaceae, a spice of the family Schisandraceae, a spice of the family Fabaceae, a spice of the family Polygonaceae, a spice of the family Brassicaceae, a spice of the family Zingiberaceae, and a spice of the family Rutaceae.
[20]

The composition described above (specifically according to any of [4] to [19]), wherein the spice is a pungent spice.
[21]

The composition described above (specifically according to any of [1] to [20]), which is a seasoning.
[22]

The composition described above (specifically according to any of [1] to [21]), wherein the gram-positive bacterium is a bacterium having an L-glutamic acid-producing ability and has one or more mutations selected from the mutations listed in Table 1 below.
[23]

The composition described above (specifically according to any of [1] to [22]), further comprising a culture of gram-positive bacterium.
[24]

A method for improving a flavor of food product, comprising a step of adding the following component (A) to an ingredient of a food product:
(A) lysozyme-treated product of cell walls of a gram-positive bacterium.
[25]

A method for producing a food product with improved flavor, comprising a step of adding the following component (A) to an ingredient of a food product:
(A) lysozyme-treated product of cell walls of a gram-positive bacterium.
[26]

The method described above (specifically according to [24] or [25]), wherein the improvement of flavor is an enhancement of spiciness and/or an impartment of kokumi.
[27]

The method described above (specifically according to any of [24] to [26]), wherein the improvement of flavor is an enhancement of pungency of spices and/or an impartment of thickness.
[28]

The method described above (specifically according to any of [24] to [27]), wherein the component (A) is a lysozyme-treated product of cells of the gram-positive bacterium or fragments thereof.
[29]

The method described above (specifically according to any of [24] to [28]), wherein the gram-positive bacterium is a bacterium belonging to the phylum Actinobacteria or Firmicutes.
[30]

The method described above (specifically according to any of [24] to [29]), wherein the gram-positive bacterium is a bacterium belonging to the phylum Actinobacteria.
[31]

The method described above (specifically according to any of [24] to [30]), wherein the gram-positive bacterium is a coryneform bacterium, a bacterium belonging to the family Bifidobacteriumceae, a bacterium belonging to the family Dermabacteraceae, a bacterium belonging to the family Bacillaceae, a bacterium belonging to the family Enterococcaceae, or a bacterium belonging to the family Lactobacilliae.
[32]

The method described above (specifically according to any of [24] to [31]), wherein the gram-positive bacterium is a bacterium of the genus Corynebacterium, a bacterium of the genus Brevibacterium, a bacterium of the genus Bifidobacterium, a bacterium of the genus Brachybacterium, a bacterium of the genus Bacillus, a bacterium of the genus Enterococcus, or a bacterium of the genus Lactobacillus.
[33]

The method described above (specifically according to any of [24] to [32]) wherein the gram-positive bacterium is Corynebacterium casei, Corynebacterium flavescens, Corynebacterium ammoniagenes (Corynebacterium stationis), Corynebacterium glutamicum, Brevibacterium casei, Bifidobacterium longum, Brachybacterium alimentarium, Bacillus subtilis, Enterococcus faecalis, Lactobacillus mali, Lactobacillus hilgardii or Lactobacillus brevis.
[34]

The method described above (specifically according to any of [24] to [33]), wherein the component (A) is added such that a concentration thereof at the time of eating or drinking is from 0.005 to 2% (w/w) in terms of dry weight of the original cells of the gram-positive bacterium.
[35]

The method described above (specifically according to any of [24] to [34]), further comprising
a step of adding the following component (B) to the ingredient of the food product:
(B) one or more components selected from the group consisting of an L-amino acid, a nucleic acid and an organic acid.
[36]

The method described above (specifically according to [35]) wherein at least the L-amino acid is added and wherein the L-amino acid is L-glutamic acid.
[37]

The method described above (specifically according to [36]), wherein the L-glutamic acid is added such that a concentration thereof at the time of eating or drinking is from 0.01 to 2% (w/w).
[38]

The method described above (specifically according to [36] or [37]), which is carried out such that a content of the L-glutamic acid in the food product is from 0.1 to 20 parts by weight of the component (A) in terms of dry weight of original cells of the gram-positive bacterium.
[39]

The method described above (specifically according to any of [24] to [38]), wherein the food product is a food product containing a spice.
[40]

The method described above (specifically according to [39]), wherein a content of the spice in the food product is from 0.01 to 2% (w/w) as a concentration thereof at the time of eating or drinking.
[41]

The method described above (specifically according to [39] or [40]), which is carried out such that the content of the spice in the food product is from 0.2 to 500 parts by weight of the component (A) in terms of dry weight of original cells of the gram-positive bacterium.
[42]

The method described above (specifically in any of [39] to [40]), wherein the spice is one or more spices selected from the group consisting of a spice of the family Lauraceae, a spice of the family Piperaceae, a spice of the family Lamiaceae, a spice of the family Apiaceae, a spice of the family Solanaceae, a spice of the family Myristicaceae, a spice of the family Alliaceae, a spice of the family Myrtaceae, a spice of the family Schisandraceae, a spice of the family Fabaceae, a spice of the family Polygonaceae, a spice of the family Brassicaceae, a spice of the family Zingiberaceae, and a spice of the family Rutaceae.
[43]

The method described above (specifically according to any of [39] to [42]), wherein the spice is a pungent spice.
[44]

The method described above (specifically according to any of [24] to [43]), wherein the gram-positive bacterium is a bacterium having an L-glutamic acid-producing ability and has one or more mutations selected from the mutations listed in Table 1 below.
[45]

The method described above (specifically according to any of [24] to [44]), further comprising a step of adding a culture of gram-positive bacterium to the ingredient of the food product.
[46]

A seasoning, comprising the following component (A):
(A) lysozyme-treated product of cell walls of a gram-positive bacterium.
[47]

The seasoning described above (specifically according to [46]), wherein the component (A) is a lysozyme-treated product of cells of the gram-positive bacterium or fragments thereof.
[48]

The seasoning described above (specifically according to [46] or [47]), further comprising the following component (B):
(B) one or more components selected from the group consisting of an L-amino acid, a nucleic acid and an organic acid.
[49]

The seasoning described above (specifically according to [48]), at least comprising the L-amino acid, wherein the L-amino acid is L-glutamic acid.
[50]

The seasoning described above (specifically according to any of [46] to [49]), further comprising a spice.
[51]

The seasoning described above (specifically according to any of [46] to [50]), wherein the gram-positive bacterium is a bacterium having an L-glutamic acid-producing ability and has one or more mutations selected from the mutations listed in Table 1 below.
[52]

The seasoning described above (specifically according to any of [46] to [51]), further comprising a culture of gram-positive bacterium.
[53]

A method of producing a composition for improving a flavor of food product, wherein the composition includes the following component (A):
(A) lysozyme-treated product of cell walls of a gram-positive bacterium;
   the method comprising
   a step of subjecting the cell walls of the gram-positive bacterium to lysozyme treatment to obtain the component (A).
[54]

The method described above (specifically according to [53]), wherein the improvement of flavor is an enhancement of spiciness and/or an impartment of kokumi.
[55]

The method described above (specifically according to [53] or [54]), wherein the improvement of flavor is an enhancement of pungency of spices and/or an impartment of thickness.
[56]

The method described above (specifically according to any of [53] to [55]), wherein the composition further comprises a spice.
[57]

The method described above (specifically according to any of [53] to [56]), wherein the cell walls of the gram-positive bacterium is subjected to the lysozyme treatment in the form of cells of the gram-positive bacterium or fragments thereof, and wherein the component (A) is a lysozyme-treated product of cells of the gram-positive bacterium or fragments thereof.
[58]

The method described above (specifically according to any of [53] to [57]), wherein a fraction of the component (A), which has a fractional molecular weight of 10000 Da or less, contains bound diaminopimeric acid.
[59]

The method described above (specifically according to [58]), wherein a content of the bound diaminopimeric acid in the fraction of the component (A), which has the fractional molecular weight of 10000 Da or less, per 100 g dry weight of the composition, excluding the dry weight of an excipient if the composition contains the excipient, is 20.0 mg or more.
[60]

The method described above (specifically according to any of [53] to [57]), wherein the composition further comprises the following component (B):
(B) one or more components selected from the group consisting of an L-amino acid, a nucleic acid and an organic acid.
[61]

The method described above (specifically according to [60]), wherein the composition comprises at least the L-amino acid, and wherein the L-amino acid is L-glutamic acid.
[62]

The method described above (specifically according to any of [53] to [61]), wherein the gram-positive bacterium is a bacterium having an L-glutamic acid-producing ability and has one or more mutations selected from the mutations listed in Table 1 below.
[63]

The method described above (specifically according to any of [53] to [62]) wherein the composition further comprises a culture of gram-positive bacterium.
[64]

The method described above (specifically according to any of [53] to [63]), wherein an amount of lysozyme used in the lysozyme treatment is from 1,950 to 39,000,000 FIP units for 1 g of dried cells of gram-positive bacterium.
[65]

A composition for improving a flavor of food product, comprising the following component (A):
(A) chitinase-treated product of cell walls of a gram-positive bacterium.
[66]

The composition described above (specifically according to [65]), wherein the improvement of flavor is an enhancement of spiciness and/or an impartment of kokumi.
[67]

The composition described above (specifically according to [65] or [66]), wherein the improvement of flavor is an enhancement of pungency of spices and/or an impartment of thickness.
[68]

The composition described above (specifically according to any of [65] to [67]), further comprising a spice.

A composition, comprising the following component (A) and a spice:
(A) chitinase-treated product of cell walls of a gram-positive bacterium.
[70]

The composition described above (specifically according to any of [65] to [69]), wherein the component (A) is a chitinase-treated product of cells of the gram-positive bacterium or fragments thereof.
[71]

The composition described above (specifically according to any of [65] to [70]), wherein the gram-positive bacterium is a bacterium belonging to the phylum Actinobacteria or Firmicutes.
[72]

The composition described above (specifically according to any of [65] to [71]), wherein the gram-positive bacterium is a bacterium belonging to the phylum Actinobacteria.
[73]

The composition described above (specifically according to any of [65] to [72]), wherein the gram-positive bacterium is a coryneform bacterium, a bacterium belonging to the family Bifidobacteriumceae, a bacterium belonging to the family Dermabacteraceae, a bacterium belonging to the family Bacillaceae, a bacterium belonging to the family Enterococcaceae, or a bacterium belonging to the family Lactobacilluseae.
[74]

The composition described above (specifically according to any of [65] to [73]), wherein the gram-positive bacterium is a bacterium of the genus Corynebacterium, a bacterium of the genus Brevibacterium, a bacterium of the genus Bifidobacterium, a bacterium of the genus Brachybacterium, a bacterium of the genus Bacillus, a bacterium of the genus Enterococcus, or a bacterium of the genus Lactobacillus.
[75]

The composition described above (specifically according to any of [65] to [74]), wherein the gram-positive bacterium is Corynebacterium casei, Corynebacterium flavescens, Corynebacterium ammoniagenes (Corynebacterium stationis), Corynebacterium glutamicum, Brevibacterium casei, Bifidobacterium longum, Brachybacterium alimentarium, Bacillus subtilis, Enterococcus faecalis, Lactobacillus mali, Lactobacillus hilgardii, or Lactobacillus brevis.
[76]

The composition described above (specifically according to any of [65] to [75]), wherein a content of the component (A) is 0.1% (w/w) or more in terms of dry weight of original cells of the gram-positive bacterium.
[77]

The composition described above (specifically according to any of [65] to [76]), wherein a fraction of the component (A), which has a fractional molecular weight of 10000 Da or less, contains bound diaminopimeric acid.
[78]

The composition described above (according to [77]), wherein a content of the bound diaminopimeric acid in the fraction of the component (A), which has the fractional molecular weight of 10000 Da or less, per 100 g dry weight of the composition, excluding the dry weight of an excipient if the composition contains the excipient, is 20.0 mg or more.
[79]

The composition described above (specifically according to any of [65] to [78]), further comprising the following component (B):
(B) one or more components selected from the group consisting of an L-amino acid, a nucleic acid and an organic acid.
[80]

The composition described above (specifically according to [79]), comprising at least the L-amino acid, wherein the L-amino acid is L-glutamic acid.
[81]

The composition described above (specifically according to [80]), wherein a content of the L-glutamic acid is from 0.1 to 20 parts by weight of the component (A) in terms of dry weight of original cells of the gram-positive bacterium.
[82]

The composition described above (specifically according to any of [68] to [81]), wherein a content of the spice is from 0.2 to 500 parts by weight of the component (A) in terms of dry weight of original cells of the gram-positive bacterium.
[83]

The composition described above (specifically according to any of [68] to [82]), wherein the spice is one or more spices selected from the group consisting of a spice of the family Lauraceae, a spice of the family Piperaceae, a spice of the family Lamiaceae, a spice of the family Apiaceae, a spice of the family Solanaceae, a spice of the family Myristicaceae, a spice of the family Alliaceae, a spice of the family Myrtaceae, a spice of the family Schisandraceae, a spice of the family Fabaceae, a spice of the family Polygonaceae, a spice of the family Brassicaceae, a spice of the family Zingiberaceae, and a spice of the family Rutaceae.
[84]

The composition described above (specifically according to any of [68] to [83]), wherein the spice is a pungent spice.
[85]

The composition described above (specifically according to any of [65] to [84]), which is a seasoning.
[86]

The composition described above (specifically according to any of [65] to [85]), wherein the gram-positive bacterium is a bacterium having an L-glutamic acid-producing ability and has one or more mutations selected from the mutations listed in Table 1 below.
[87]

The composition described above (specifically according to any of [65] to [86]), further comprising a culture of gram-positive bacterium.
[88]

A method for improving a flavor of food product, comprising
a step of adding the following component (A) to an ingredient of a food product:
(A) chitinase-treated product of cell walls of a gram-positive bacterium.
[89]

A method for producing a food product with improved flavor, comprising
a step of adding the following component (A) to an ingredient of a food product:
(A) chitinase-treated product of cell walls of a gram-positive bacterium.
[90]

The method described above (specifically according to [88] or [89]), wherein the improvement of flavor is an enhancement of spiciness and/or an impartment of kokumi.
[91]

The method described above (specifically according to any of [88] to [90]), wherein the improvement of flavor is an enhancement of pungency of spices and/or an impartment of thickness.
[92]

The method described above (specifically according to any of [88] to [91]), wherein the component (A) is a chitinase-treated product of cells of the gram-positive bacterium or fragments thereof.
[93]

The method described above (specifically according to any of [88] to [92]), wherein the gram-positive bacterium is a bacterium belonging to the phylum Actinobacteria or Firmicutes.
[94]

The method described above (specifically according to any of [88] to [93]), wherein the gram-positive bacterium is a bacterium belonging to the phylum Actinobacteria.
[95]

The method described above (specifically according to any of [88] to [94]), wherein the gram-positive bacterium is a coryneform bacterium, a bacterium belonging to the family Bifidobacteriumceae, a bacterium belonging to the family Dermabacteraceae, a bacterium belonging to the family Bacillaceae, a bacterium belonging to the family Enterococcaceae, or a bacterium belonging to the family Lactobacilluseae.
[96]

The method described above (specifically according to any of [88] to [95]), wherein the gram-positive bacterium is a bacterium of the genus Corynebacterium, a bacterium of the genus Brevibacterium, a bacterium of the genus Bifidobacterium, a bacterium of the genus Brachybacterium, a bacterium of the genus Bacillus, a bacterium of the genus Enterococcus, or a bacterium of the genus Lactobacillus.
[97]

The method described above (specifically according to any of [88] to [96]), wherein the gram-positive bacterium is Corynebacterium casei, Corynebacterium flavescens, Corynebacterium ammoniagenes (Corynebacterium stationis), Corynebacterium glutamicum, Brevibacterium casei, Bifidobacterium longum, Brachybacterium alimentarium, Bacillus subtilis, Enterococcus faecalis, Lactobacillus mali, Lactobacillus hilgardii, or Lactobacillus brevis.
[98]

The method described above (specifically according to any of [88] to [97]), wherein the component (A) is added such that a concentration thereof at the time of eating or drinking is from 0.005 to 2% (w/w) in terms of dry weight of original cells of the gram-positive bacterium.
[99]

The method described above (specifically according to any of [88] to [98]), further comprising a step of adding the following component (B) to the ingredient of the food product:
(B) one or more components selected from the group consisting of an L-amino acid, a nucleic acid and an organic acid.
[100]

The method described above (specifically according to [99]), wherein at least the L-amino acid is added, and wherein the L-amino acid is L-glutamic acid.
[101]

The method described above (specifically according to [100]), wherein the L-glutamic acid is added such that a concentration thereof at the time of eating or drinking is from 0.01 to 2% (w/w).
[102]

The method described above (specifically according to [100] or [101]), which is carried out such that a content of the L-glutamic acid in the food product is from 0.1 to 20 parts by weight of the component (A) in terms of dry weight of original cells of the gram-positive bacterium.
[103]

The method described above (specifically according to any of [88] to [102]), wherein the food product is a food product containing a spice.
[104]

The method described above (specifically according to [103]), wherein a content of the spice in the food product is from 0.01 to 2% (w/w) as a concentration thereof at the time of eating or drinking.
[105]

The method described above (specifically according to [103] or [104]), which is carried out such that the content of the spice in the food product is from 0.2 to 500 parts by weight of the component (A) in terms of dry weight of original cells of the gram-positive bacterium.
[106]

The method described above (specifically according to any of [103] to [105]), wherein the spice is one or more spices selected from the group consisting of a spice of the family Lauraceae, a spice of the family Piperaceae, a spice of the family Lamiaceae, a spice of the family Apiaceae, a spice of the family Solanaceae, a spice of the family Myristicaceae, a spice of the family Alliaceae, a spice of the family Myrtaceae, a spice of the family Schisandraceae, a spice of the family Fabaceae, a spice of the family Polygonaceae, a spice of the family Brassicaceae, a spice of the family Zingiberaceae, and a spice of the family Rutaceae.
[107]

The method described above (specifically according to any of [103] to [106]), wherein the spice is a pungent spice.
[108]

The method described above (specifically according to any of [88] to [107]), wherein the gram-positive bacterium is a bacterium having an L-glutamic acid-producing ability and has one or more mutations selected from the mutations listed in Table 1 below.
[109]

The method described above (specifically according to any of [88] to [108]), further comprising a step of adding a culture of gram-positive bacterium to the ingredient of the food product.
[110]

A seasoning, comprising the following component (A):
(A) chitinase-treated product of cell walls of a gram-positive bacterium.
[111]

The seasoning described above (specifically according to [110]), wherein the component (A) is a chitinase-treated product of cells of the gram-positive bacterium or fragments thereof.
[112]

The seasoning described above (specifically according to [110] or [111]), further comprising the following component (B):
(B) one or more components selected from the group consisting of an L-amino acid, a nucleic acid and an organic acid.
[113]

The seasoning described above (specifically according to [112]), at least comprising the L-amino acid, wherein the L-amino acid is L-glutamic acid.
[114]

The seasoning described above (specifically according to any of [110] to [113]), further comprising a spice.
[115]

The seasoning described above (specifically according to any of [110] to [114]), wherein the gram-positive bacterium is a bacterium having an L-glutamic acid-producing ability and has one or more mutations selected from the mutations listed in Table 1 below.
[116]

The seasoning described above (specifically according to any of [110] to [115]), further comprising a culture of gram-positive bacterium.
[117]

A method of producing a composition for improving a flavor of food product, wherein the composition includes the following component (A):
(A) chitinase-treated product of cell walls of a gram-positive bacterium;
   the method comprising
   a step of subjecting the cell walls of the gram-positive bacterium to chitinase treatment to obtain the component (A).
[118]

The method described above (specifically according to [117]), wherein the improvement of flavor is an enhancement of spiciness and/or an impartment of kokumi.
[119]

The method described above (specifically according to [117] or [118]), wherein the improvement of flavor is an enhancement of pungency of spices and/or an impartment of thickness.
[120]

The method described above (specifically according to any of [117] to [119]), wherein the composition further comprises a spice.
[121]

The method described above (specifically according to any of [117] to [120]), wherein the cell walls of the gram-positive bacterium are subjected to the chitinase treatment in the form of cells of the gram-positive bacterium or fragments thereof, and wherein the component (A) is a chitinase-treated product of cells of the gram-positive bacterium or fragments thereof.
[122]

The method described above (specifically according to any of [117] to [121]), wherein a fraction of the component (A), which has a fractional molecular weight of 10000 Da or less, contains bound diaminopimeric acid.
[123]

The method described above (specifically according to [122]), wherein a content of the bound diaminopimeric acid in the fraction of the component (A), which has the fractional molecular weight of 10000 Da or less, per 100 g dry weight of the composition, excluding the dry weight of an excipient if the composition contains the excipient, is 20.0 mg or more.
[124]

The method described above (specifically according to any of [117] to [123]), wherein the composition further comprises the following component (B):
(B) one or more components selected from the group consisting of an L-amino acid, a nucleic acid and an organic acid.
[125]

The method described above (specifically according to [124]), wherein the composition comprises at least the L-amino acid, and wherein the L-amino acid is L-glutamic acid.
[126]

The method described above (specifically according to any of [117] to [125]), wherein the gram-positive bacterium is a bacterium having an L-glutamic acid-producing ability and has one or more mutations selected from the mutations listed in Table 1 below.
[127]

The method described above (specifically according to any of [117] to [126]), wherein the composition further comprises a culture of gram-positive bacterium.
[128]

The method described above (specifically according to any of [117] to [129]), wherein an amount of chitinase used in the chitinase treatment is from 1,950 to 39,000,000 FIP units for 1 g of dried cells of the gram-positive bacterium.

### Advantageous Effects of the Invention

According to the present invention, the flavor of food products can be improved.

### EMBODIMENTS FOR CARRYING OUT THE INVENTION

### <1> Active Ingredients

In the present invention, the following component (A) is used as an active ingredient:
(A) lysozyme-treated product of cell walls of gram-positive bacterium.

Component (A) above is also referred to as the ‘active ingredient’.

The use of active ingredients can improve the flavor of food product, i.e. the effect of improving the flavor of food product can be obtained. The effect is also referred to as “flavor-improving effect”. In other words, the active ingredient has the function of improving the flavor of the food product. This function is also referred to as the “flavor-improving function”. The active ingredient may have a higher flavor-improving function compared to the cell walls of gram-positive bacterium. In other words, a flavor-improving function improved by the lysozyme treatment of the gram-positive bacterial cell wall may provided. ‘The active ingredient has a higher flavor-improving function compared to the cell walls of gram-positive bacterium’ means that the flavor-improving effect obtained by utilising a certain amount of the active ingredient (which may be, for example, the amount of the active ingredient added in the method of the present invention, as described below) is higher than that obtained by utilising the cell walls of gram-positive bacterium at the amount which is used in the preparation of that amount of active ingredient. The improvement of the flavor of the food product is also referred to simply as “flavor improvement”. Specifically, the utilisation of the active ingredient improves the flavor of the food product compared to the case where the active ingredient is not utilised. Thus, the active ingredient may be utilised to improve the flavor of the food product.

The active ingredient may also be utilised to produce a food product with an improved flavor. Thus, the active ingredient may be utilised in the production of a food product (specifically, in the production of a food product with an improved flavor).

Flavor includes taste and aroma. In other words, the utilisation of the active ingredient may improve the taste and/or the aroma. The term “aroma” here may mean the aroma felt from the throat to the nasal cavity when eating or drinking the food product (i.e. retronasal aroma) and/or the aroma directly smelled through the nose (i.e. orthonasal aroma). “Aroma” herein may mean, in particular, the aroma felt from the throat to the nasal cavity when the food product is consumed (i.e. retronasal aroma).

Flavor improvements include an enhancement of spiciness and an impartment of kokumi. Flavor improvements include, in particular, the enhancement of spiciness. By utilising the active ingredient, one flavor may be improved alone, or two or more flavors may be improved in combination. In other words, by utilising an active ingredient, for example, an enhancement of spiciness and/or an impartment of kokumi may be achieved. The active ingredient may have a higher flavor-improving function with respect to one or more flavors compared to the cell walls of gram-positive bacterium. The active ingredient may, for example, have a higher flavor-improving function compared to the cell walls of gram-positive bacterium for some or all of the flavors improved by utilising the active ingredient. The active ingredient may, for example, have a higher spiciness-enhancing and/or kokumi-imparting function compared to the cell walls of gram-positive bacterium.

“Spiciness” may mean the sensation felt due to the presence of spice. Spiciness may also be referred to as Spice Flavor, Spice aroma, Pungency, Hotness or Burningness. “Spiciness” may specifically mean the sensation felt due to the presence of spice when eating or drinking a food product containing a spice. “Spiciness” may be used interchangeably with “spice flavor”. Examples of spiciness include spice taste and spice aroma. Examples of spiciness specifically include the pungency of spice. “Enhancement of the spiciness” is not limited to enhancing the spiciness of a food product with a spiciness (e.g. a food product containing a spice), but may also include imparting a spiciness to a food product without a spiciness (e.g. a food product that does not contain a spice). For example, the active ingredient and the spice can be used together to impart a spiciness (specifically, an enhanced spiciness compared to a food product that does not utilise the active ingredient) to a food product that does not have a spiciness.

The term “kokumi” refers to sensation that cannot be expressed by the five basic tastes (sweet taste, salty taste, sour taste, bitter taste and umami), and specifically includes not only the basic tastes, but also the tastes around the basic tastes (marginal tastes/marginal flavor) such as thickness, growth (mouthfulness), continuity and harmony. The term “impartment of kokumi” may mean enhancement of basic taste and/or enhancement or impartment of the marginal tastes and flavors around the basic taste, such as thickness, growth, continuity, harmony. Examples of the “impartment of kokumi” may also include imparting or enhancing flavors such as complexity, maturity, richness, meatiness, milkiness, juiceiness, body (e.g. sugar-like body, wine body), etc. The “impartment of kokumi” includes, in particular, the impartment of thickness.

Flavor (e.g. spiciness or kokumi) may be classified, for example, into initial taste, middle taste and aftertaste. For flavor, “initial taste”, “middle taste” and “aftertaste” mean, in the case of liquids (for liquid foods), the flavor perceived from 0 to 1 second, from 1 to 3 seconds and from 3 to 5 seconds, respectively, after consumption (after the food is put into the mouth). In addition, the terms “initial taste”, “middle taste” and “aftertaste” for flavor mean, in the case of solids (in the case of solid foods), the flavor perceived from 0 to 4 seconds, from 4 to 10 seconds and from 10 to 15 seconds after consumption (after the food is put into the mouth), respectively. In the present invention, “solid” refers to a form other than liquid and includes pastes, gels, and the like. By utilising the active ingredient, for example, the flavor of the initial taste, the middle taste, the aftertaste or a combination thereof may be improved. That is, by utilising the active ingredient, specifically, for example, the spiciness of the initial taste, the spiciness of the middle taste, the spiciness of the aftertaste, or a combination thereof, may be enhanced. Also, by utilising the active ingredient, specifically, for example, the kokumi of the initial taste, the kokumi of the middle taste, the kokumi of the aftertaste, or a combination thereof, may be imparted. By utilising the active ingredient, in particular, kokumi of the initial taste (e.g. thickness of the initial taste) may be imparted. The active ingredient may, for example, have the function of imparting a high kokumi (e.g. thickness of the initial taste) compared to the cell walls of gram-positive bacterium.

Flavor (e.g. spiciness or kokumi) can be measured and compared, e.g. by sensory evaluation by expert panelists.

The active ingredient may be used to improve flavor or to produce a food product in the manner described in the below-described methods of the present invention.

The active ingredient can be produced by subjecting the cell walls of gram-positive bacterium to lysozyme treatment.

In other words, the present description discloses a method of producing an active ingredient, comprising a step of subjecting the cell walls of gram-positive bacterium to lysozyme treatment. The step is also referred to as the “lysozyme treatment step”. The lysozyme treatment step may specifically be a step wherein the cell walls of gram-positive bacterium are subjected to lysozyme treatment to obtain the active ingredient.

The cell walls of gram-positive bacterium may be subjected to lysozyme treatment in any form. The cell walls of gram-positive bacterium may, for example, be subjected to lysozyme treatment in the form of cells of the gram-positive bacterium or fragments thereof. In other words, the cell walls of gram-positive bacterium that are subjected to lysozyme treatment are specifically the cells of gram-positive bacterium or fragments thereof. In other words, the cell walls of gram-positive bacterium that are subjected to lysozyme treatment are specifically the cell walls that comprise (i.e. are contained in) the cells of gram-positive bacterium or the cell walls that comprise (i.e. are contained in) the fragments of the bacterium. In other words, the active ingredient is specifically a lysozyme-treated product of cells of a gram-positive bacterium or a lysozyme-treated product of a fragment of the cells. In other words, the active ingredient is specifically a lysozyme-treated product of the cell wall comprising (i.e. contained in) the cells of a gram-positive bacterium or a lysozyme-treated product of the cell wall comprising (i.e. contained in) a fragment of the cells. The cell walls of gram-positive bacterium that are subjected to lysozyme treatment are, in particular, the cells of gram-positive bacterium. This means that the active ingredient is, in particular, a lysozyme-treated product of the cells of the gram-positive bacterium.

The gram-positive bacterium is not limited. Examples of the gram-positive bacterium include a bacterium belonging to the phylum Actinobacteria and the phylum Firmicutes. Examples of the gram-positive bacterium include, in particular, a bacterium belonging to the phylum Actinobacteria.

Examples of a bacterium belonging to the phylum Actinobacteria include a coryneform bacterium, a bacterium belonging to the family Bifidobacteriumceae and a bacterium belonging to the family Dermabacteraceae. Examples of a bacterium belonging to the phylum Actinobacteria include, in particular, a coryneform bacterium.

Examples of the coryneform bacterium include a bacterium of the genus Corynebacterium, a bacterium of the genus Brevibacterium and a bacterium of the genus Microbacterium.

Specifically, examples of the coryneform bacterium include the following species.
Corynebacterium acetoacidophilum
Corynebacterium acetoglutamicum
Corynebacterium alkanolyticum
Corynebacterium callunae
Corynebacterium casei
Corynebacterium crenatum
Corynebacterium flavescens
Corynebacterium glutamicum
Corynebacterium lilium
Corynebacterium melassecola
Corynebacterium thermoaminogenes (Corynebacterium efficiens)
Corynebacterium herculis
Brevibacterium casei
Brevibacterium divaricatum (Corynebacterium glutamicum)
Brevibacterium flavum (Corynebacterium glutamicum)
Brevibacterium immariophilum
Brevibacterium lactofermentum (Corynebacterium glutamicum)
Brevibacterium roseum
Brevibacterium saccharolyticum
Brevibacterium thiogenitalis
Corynebacterium ammoniagenes (Corynebacterium stationis)
Brevibacterium album
Brevibacterium cerinum
Microbacterium ammoniaphilum

More specifically, examples of the coryneform bacterium include the following strains.
Corynebacterium acetoacidophilum ATCC 13870
Corynebacterium acetoglutamicum ATCC 15806
Corynebacterium alkanolyticum ATCC 21511
Corynebacterium callunae ATCC 15991
Corynebacterium casei JCM 12072
Corynebacterium crenatum AS1.542
Corynebacterium flavescens ATCC 10340 (NBRC 14136)
Corynebacterium glutamicum ATCC 13020, ATCC 13032, ATCC 13060, ATCC 13869, FERM BP-734
Corynebacterium lilium ATCC 15990
Corynebacterium melassecola ATCC 17965
Corynebacterium efficiens (Corynebacterium thermoaminogenes) AJ12340 (FERM BP-1539)
Corynebacterium herculis ATCC 13868
Brevibacterium casei ATCC 35513 (DSM 20657)
Brevibacterium divaricatum (Corynebacterium glutamicum) ATCC 14020
Brevibacterium flavum (Corynebacterium glutamicum) ATCC 13826, ATCC 14067, AJ12418 (FERM BP-2205)
Brevibacterium immariophilum ATCC 14068
Brevibacterium lactofermentum (Corynebacterium glutamicum) ATCC 13869
Brevibacterium roseum ATCC 13825.
Brevibacterium saccharolyticum ATCC 14066
Brevibacterium thiogenitalis ATCC 19240
Corynebacterium ammoniagenes (Corynebacterium stationis) ATCC 6871, ATCC 6872
Brevibacterium album ATCC 15111
Brevibacterium cerinum ATCC 15112
Microbacterium ammoniaphilum ATCC 15354

Examples of the coryneform bacterium include, in particular, a bacterium of the genus Corynebacterium and a bacterium of the genus Brevibacterium. Examples of the bacterium of the genus Corynebacterium include, in particular, Corynebacterium casei such as Corynebacterium casei JCM 12072 and Corynebacterium flavescens such as Corynebacterium flavescens ATCC 10340. Examples of the bacterium of the genus Brevibacterium, in particular, include Brevibacterium casei such as Brevibacterium casei ATCC 35513.

Note that the bacterium of the genus Corynebacterium also includes a bacterium previously classified in the genus Brevibacterium, but now integrated into the genus Corynebacterium (Int. J. Syst. Bacteriol., 41, 255 (1991)). Corynebacterium stationis also includes bacterium previously classified as Corynebacterium ammoniagenes but reclassified to Corynebacterium stationis through 16S rRNA sequencing and other analyses (Int. J. Syst. Evol. Microbiol. 60, 874-879 (2010)).

Examples of the bacterium belonging to the family Bifidobacteriumceae include a bacterium of the genus Bifidobacterium. Examples of the bacterium of the genus Bifidobacterium include Bifidobacterium longum, Bifidobacterium breve, Bifidobacterium bifidum, Bifidobacterium adolescentis, Bifidobacterium angulatum, Bifidobacterium dentium, Bifidobacterium pseudocatenulatum, Bifidobacterium animalis, Bifidobacterium pseudolongum, Bifidobacterium thermophilum. Examples of the bacterium of the genus Bifidobacterium include, in particular, Bifidobacterium longum. Examples of Bifidobacterium longum specifically include ATCC 15697, ATCC 15707, ATCC 25962, ATCC 15702, ATCC 27533, BG7, DSM 24736, SBT 2928, NCC 490 (CNCM I-2170), NCC 2705 (CNCM I-2618). Examples of Bifidobacterium breve specifically include ATCC 15700, B632 (DSM 24706), Bb99 (DSM 13692), ATCC 15698, DSM 24732, UCC 2003, YIT 4010, YIT 4064, BBG-001, BR-03, C50 and R0070. Examples of Bifidobacterium bifidum specifically include ATCC 29521, OLB6378 and BF-1. Examples of Bifidobacterium adolescentis specifically include ATCC 15703. Examples of Bifidobacterium dentium specifically include DSM 20436. Examples of Bifidobacterium pseudocatenulatum specifically include ATCC 27919. Examples of Bifidobacterium animalis specifically include DSM 10140, Bb-12, DN-173 010, GCL 2505 and CNCM I-3446. Examples of Bifidobacterium pseudolongum specifically include JCM 5820 and ATCC 25526. Examples of Bifidobacterium thermophilum specifically include ATCC 25525.

Examples of the bacterium belonging to the family Dermabacteraceae include a bacterium of the genus Brachybacterium. Examples of the bacterium of the genus Brachybacterium include Brachybacterium alimentarium and Brachybacterium tyrofermentans. Examples of Brachybacterium alimentarium specifically include ATCC 700067 (NBRC 16118). Examples of Brachybacterium tyrofermentans specifically include DSM 10673.

Examples of the bacterium belonging to the phylum Firmicutes include a bacterium belonging to the family Bacillaceae, a bacterium belonging to the family Enterococcaceae and a bacterium belonging to the family Lactobacilluseae.

Examples of the bacterium belonging to the family Bacillaceae include a bacterium of the genus Bacillus. Examples of the bacterium of the genus Bacillus include Bacillus subtilis, Bacillus amyloliquefaciens, Bacillus pumilus, Bacillus licheniformis, Bacillus megaterium, Bacillus brevis, Bacillus polymixa, Bacillus stearothermophilus, and Bacillus velezensis. Examples of the bacterium of the genus Bacillus include, in particular, Bacillus subtilis. Examples of Bacillus subtilis specifically include strains 168 Marburg (ATCC 6051, JCM 1465) and PY79 (Plasmid, 1984, 12, 1-9). Examples of Bacillus amyloliquefaciens specifically include strains T (ATCC 23842), N (ATCC 23845), AJ11708 (NITE BP-02609) and FZB42 (DSM 23117).

Examples of the bacterium belonging to the family Enterococcaceae include a bacterium of the genus Enterococcus. Examples of the bacterium of the genus Enterococcus include Enterococcus faecalis and Enterococcus faecium. Examples of the bacterium of the genus Enterococcus include, in particular, Enterococcus faecalis. Examples of Enterococcus faecalis specifically include ATCC 19433. Examples of Enterococcus faecium specifically include ATCC 19434.

Examples of the bacterium belonging to the family Lactobacilliae include a bacterium of the genus Lactobacillus. Examples of the bacterium of the genus Lactobacillus include Lactobacillus mali, Lactobacillus hilgardii, Lactobacillus brevis, Lactobacillus delbrueckii, Lactobacillus casei, Lactobacillus paracasei, Lactobacillus gasseri and Lactobacillus acidophilus. Examples of the bacterium of the genus Lactobacillus include, in particular, Lactobacillus mali, Lactobacillus hilgardii and Lactobacillus brevis. Examples of Lactobacillus mali specifically include NBRC 102159 (ATCC 27053). Examples of Lactobacillus hilgardii specifically include NBRC 15886 (ATCC 8290) Examples of Lactobacillus brevis specifically include JCM 1102 (ATCC 27305).

These strains are available, for example, from the American Type Culture Collection, address 12301 Parklawn Drive, Rockville, Maryland 20852 P.O. Box 1549, Manassas, VA 20108, United States of America. The registration number corresponding to each strain has been assigned, which can be used to access the distribution (http://www.atcc.org/). The registration number corresponding to each strain can be found in the catalogue of the American Type Culture Collection. These strains can also be obtained, for example, from the depository where each strain was deposited.

The gram-positive bacterium may be modified as appropriate. In other words, the gram-positive bacterium may be a modified strain derived from any of the strains exemplified above. Examples of the modified strain specifically include a modified strain derived from Corynebacterium casei JCM 12072. The purpose of the modification is not restricted. Modifications include modifications to impart or enhance target substance-producing ability. Modified strains may, for example, be bred by artificial modification. The Artificial modification may be modification by genetic engineering or modification by mutation treatment. Examples of the mutation treatment include X-ray irradiation, UV irradiation and treatment with mutagens such as N-methyl-N′-nitro-N-nitrosoguanidine (MNNG), ethyl methane sulphonate (EMS), and methyl methane sulphonate (MMS). The modified strain may also be naturally occurring, for example, during the use of gram-positive bacterium. Examples of such modified strains include, for example, a mutant strain that arises naturally during the culture of gram-positive bacterium. The modified strain may be constructed by a single modification or by a combination of two or more modifications.

The gram-positive bacterium may have target substance-producing ability.

“Target substance-producing ability” means the ability to produce a target substance. In other words, “bacterium having target substance-producing ability” means a bacterium having the ability to produce the target substance. “Bacterium having target substance-producing ability” may specifically mean a bacterium having the ability to accumulate the target substance, when cultured in a culture medium, in the culture medium and/or within the cells to the extent that the target substance can be produced and recovered. If the bacterium having target substance-producing ability is a modified strain, the bacterium having target substance-producing ability may be a bacterium that is able to accumulate the target substance in the culture medium and/or within the cells in greater quantities than a non-modified strain. The non-modified strain may be a wild strain and a parental strain. The bacterium having target substance-producing ability may in particular be a bacterium that is able to accumulate the target substance in the culture medium. The bacterium having target substance-producing ability may be a bacterium that is able to accumulate the target substance in the culture medium in amounts of preferably 0.5 g/L or more, more preferably 1.0 g/L or more. The bacterium having target substance-producing ability may have an ability to produce only one target substance, or may have an ability to produce two or more target substances.

The target substance is not restricted to those that can be produced by the culture of gram-positive bacterium. Examples of the target substances include an ingredient used in food products. Examples of the target substance include an L-amino acid, a nucleic acid and an organic acid.

Examples of the L-amino acid include a basic amino acids such as L-lysine, L-ornithine, L-arginine, L-histidine, and L-citrulline; an aliphatic amino acid such as L-isoleucine, L-alanine, L-valine, L-leucine and glycine; a hydroxymonoaminocarboxylic acid such as L-threonine and L-serine; a cyclic amino acid such as L-proline; an aromatic amino acid such as L-phenylalanine, L-tyrosine, L-tryptophan; a sulphur-containing amino acid such as L-cysteine, L-cystine and L-methionine; an acidic amino acid such as L-glutamic acid and L-aspartic acid; an amino acid such as amino acids with amide groups in the side chain of L-glutamine, L-aspartic acid, and the like.

Examples of a nucleic acid include a purine substance. Examples of the purine substance include a purine nucleoside and a purine nucleotide. Examples of the purine nucleoside include inosine, guanosine, xanthosine and adenosine. Examples of the purine nucleotide include 5′-phosphate esters of purine nucleosides. Examples of the 5′-phosphate esters of purine nucleosides include inosinic acid (inosine-5′-phosphate; IMP), guanylic acid (guanosine-5′-phosphate; GMP), xanthate (xanthosine-5′-phosphate; XMP) and adenylic acid (adenosine-5′-phosphate; AMP). Examples of the purine substance include, in particular, inosine and guanosine. Examples of the purine substance include, more particularly, inosine.

Examples of the organic acid include a carboxylic acid. Examples of the carboxylic acid include a monocarboxylic acid and a dicarboxylic acid. Examples of the monocarboxylic acid include a monocarboxylic acid with three to eight carbons (C₃-C₈ monocarboxylic acids). A specific example of a monocarboxylic acid is pyruvic acid. Examples of the dicarboxylic acid include dicarboxylic acids (C₃-C₈ dicarboxylic acids) with three to eight carbons. Examples of the dicarboxylic acid specifically include α-ketoglutaric acid (a-KG; also known as 2-oxoglutaric acid), malic acid, fumaric acid, succinic acid, itaconic acid, malonic acid, adipic acid, glutaric acid, pimelic acid, and suberic acid.

Where the target substance can form salts, the target substance may be produced and/or used as a free form, produced and/or used as a salt, or produced and/or used as a combination thereof. In other words, the term “target substance” may mean the target substance in a free form, or a salt thereof, or a combination thereof, unless otherwise stated. If the target substance can form hydrates, the target substance may be produced and/or used as an unhydrate, produced and/or used as a hydrate, or produced and/or used as a combination thereof. In other words, the term “target substance” (e.g. “target substance in a free from” or “salt of target substance”) may encompass non-hydrated and hydrated substances, unless otherwise stated. The target substance may be in any form, such as ionic, at the time of use. The amount of the target substance (e.g. content (concentration) or amount used) shall be calculated based on the mass of the salt or hydrate converted to the mass of the free body in equal molarity, in the case where the target substance forms a salt or hydrate.

The salt can be selected according to various conditions, such as the intended use of the target substance. For example, if the target substance is to be used in oral intake applications, a salt that can be taken orally can be selected. Examples of the salt for acidic groups such as carboxyl groups include an ammonium salt, a salt with an alkali metal such as sodium and potassium, a salt with an alkaline earth metal such as calcium and magnesium, an aluminium salts, a zinc salt, a salt with an organic amine such as triethylamine, ethanolamine, morpholine, pyrrolidine, piperidine, piperazine and dicyclohexylamine, and a salt with a basic amino acid such as arginine and lysine. Examples of the salt for basic groups such as amino groups include a salt with an inorganic acid such as hydrochloric acid, sulphuric acid, phosphoric acid, nitric acid, and hydrobromic acid, a salt with an organic carboxylic acid such as acetic acid, citric acid, benzoic acid, maleic acid, fumaric acid, tartaric acid, succinic acid, tannic acid, butyric acid, hibenzic acid, pamoic acid, enanthic acid, decanoic acid, theocuric acid, salicylic acid, lactic acid, oxalic acid, mandelic acid, malic acid, methylmalonic acid and adipic acid, and a salt with an organic sulphonic acid such as methanesulphonic acid, benzenesulphonic acid and p-toluenesulphonic acid. For example, examples of a salt of L-glutamic acid include, in particular, sodium L-glutamate (e.g. monosodium L-glutamate; MSG) and ammonium L-glutamate (e.g. monoammonium L-glutamate). One salt may be used, or a combination of two or more salts may be used.

The gram-positive bacterium may inherently have target substance-producing ability or may be modified to have target substance-producing ability. The gram-positive bacterium having target substance-producing ability can be obtained, for example, by imparting target substance-producing ability to the gram-positive bacterium as described above, or by enhancing the target substance-producing ability of the gram-positive bacterium as described above.

Methods for imparting or enhancing target substance-producing ability are not particularly restricted. The methods for imparting or enhancing target substance-producing ability can, for example, utilise known methods. The method for imparting or enhancing target substance-producing ability can be performed, for example, by mutation or genetic engineering methods. Methods for imparting or enhancing L-amino acid—(e.g. L-glutamic acid) producing ability are disclosed, for example, in WO 2006/070944, WO 2015/060391 and WO2018/030507. Methods for imparting or enhancing nucleic acid-producing ability are disclosed, for example, in WO 2015/060391. Also, for example, the imparting or enhancing of L-glutamic acid-producing ability can be performed according to the procedures described in the Examples.

Bacterium having L-glutamic acid-producing ability (also referred to as “L-glutamic acid-producing bacterium”) include bacterium with “specific mutations”.

“Specific mutations” include the mutations listed in Table 1. The mutations shown in Table 1 comprise 135 mutations, from mutations A-1 to A-135, and 92 mutations, from mutations B-1 to B-92. Mutations A-1 to A-135 are also referred to as “Group A mutations”. Mutations B-1 to B-92 are also referred to as “Group B mutations”.

TABLE 1

| | | Pre- | Post- |
|---|---|---|---|
| | Genomic | mutation | mutation |
| No. | position | base | base |
| A-1 | 78,486 | C | T |
| A-2 | 83,592 | G | A |
| A-3 | 87,955 | C | T |
| A-4 | 90,041 | C | T |
| 4-5 | 186,221 | C | T |
| A-6 | 193,010 | C | T |
| A-7 | 196,531 | C | T |
| A-8 | 225,429 | C | T |
| A-9 | 297,920 | G | A |
| A-10 | 320,354 | C | T |
| A-11 | 335,878 | C | T |
| A-12 | 341,763 | C | T |
| A-13 | 346,969 | C | T |
| A-14 | 349,856 | C | T |
| A-15 | 356,232 | C | T |
| A-16 | 357,008 | C | T |
| A-17 | 366,674 | G | A |
| A-18 | 369,871 | G | A |
| A-19 | 377,420 | G | A |
| A-20 | 378,652 | G | A |
| A-21 | 432,252 | C | A |
| A-22 | 439,021 | G | A |
| A-23 | 440,764 | G | A |
| A-24 | 454,682 | G | A |
| A-25 | 458,729 | G | A |
| A-26 | 470,562 | G | A |
| A-27 | 471,288 | G | A |
| A-28 | 472,023 | G | A |
| A-29 | 504,885 | G | A |
| A-30 | 505,785 | G | A |
| A-31 | 514,371 | G | A |
| A-32 | 518,684 | G | A |
| A-33 | 521,126 | G | A |
| A-34 | 524,551 | G | A |
| A-35 | 660,841 | C | T |
| A-36 | 732,121 | C | T |
| A-37 | 787,055 | C | T |
| A-38 | 806,047 | C | T |
| A-39 | 872,482 | G | A |
| A-40 | 878,069 | C | T |
| A-41 | 903,037 | C | T |
| A-42 | 922,802 | C | T |
| A-43 | 948,145 | C | T |
| A-44 | 955,819 | C | T |
| A-45 | 968,915 | C | T |
| A-46 | 973,013 | C | T |
| A-47 | 974,797 | C | T |
| A-48 | 994,815 | C | T |
| A-49 | 1,000,498 | C | T |
| A-50 | 1,019,704 | C | T |
| A-51 | 1,049,052 | C | T |
| A-52 | 1,069,322 | C | T |
| A-53 | 1,070,554 | C | T |
| A-54 | 1,131,016 | C | T |
| A-55 | 1,138,639 | C | T |
| A-56 | 1,162,588 | C | T |
| A-57 | 1,193,273 | C | T |
| A-58 | 1,203,146 | C | T |
| A-59 | 1,222,633 | C | T |
| A-60 | 1,226,969 | G | A |
| A-61 | 1,264,895 | G | A |
| A-62 | 1,268,790 | G | A |
| A-63 | 1,279,676 | G | A |
| A-64 | 1,363,909 | T | C |
| A-65 | 1,387,476 | G | A |
| A-66 | 1,401,171 | G | A |
| A-67 | 1,416,228 | C | T |
| A-68 | 1,420,034 | C | T |
| A-69 | 1,447,494 | C | T |
| A-70 | 1,448,318 | C | T |
| A-71 | 1,448,776 | C | T |
| A-72 | 1,451,922 | C | T |
| A-73 | 1,466,961 | C | T |
| A-74 | 1,503,736 | C | T |
| A-75 | 1,504,207 | C | T |
| A-76 | 1,505,998 | C | T |
| A-77 | 1,507,027 | C | T |
| A-78 | 1,544,310 | C | T |
| A-79 | 1,554,973 | C | T |
| A-80 | 1,558,509 | C | T |
| A-81 | 1,562,459 | C | T |
| A-82 | 1,572,716 | C | T |
| A-83 | 1,594,314 | C | T |
| A-84 | 1,602,545 | C | T |
| A-85 | 1,659,808 | C | T |
| A-86 | 1,682,132 | C | T |
| A-87 | 1,689,863 | C | T |
| A-88 | 1,744,963 | C | T |
| A-89 | 1,784,642 | C | T |
| A-90 | 1,814,866 | C | T |
| A-91 | 1,829,145 | C | T |
| A-92 | 1,852,511 | G | A |
| A-93 | 1,861,170 | G | A |
| A-94 | 1,902,133 | G | A |
| A-95 | 1,916,048 | C | T |
| A-96 | 1,917,434 | C | T |
| A-97 | 1,938,271 | C | T |
| A-98 | 1,949,357 | G | T |
| A-99 | 1,954,368 | C | T |
| A-100 | 1,967,997 | C | T |
| A-101 | 1,975,599 | C | T |
| A-102 | 2,141,466 | C | T |
| A-103 | 2,308,064 | C | T |
| A-104 | 2,310,428 | C | T |
| A-105 | 2,354,420 | C | T |
| A-106 | 2,449,270 | T | C |
| A-107 | 2,449,278 | C | A |
| A-108 | 2,449,291 | G | C |
| A-109 | 2,449,318 | G | A |
| A-110 | 2,496,945 | C | T |
| A-111 | 2,505,022 | C | T |
| A-112 | 2,505,285 | C | T |
| A-113 | 2,525,513 | G | A |
| A-114 | 2,565,856 | C | T |
| A-115 | 2,601,306 | G | A |
| A-116 | 2,615,688 | G | A |
| A-117 | 2,650,740 | G | A |
| A-118 | 2,653,259 | G | A |
| A-119 | 2,663,827 | G | A |
| A-120 | 2,667,322 | G | A |
| A-121 | 2,674,077 | G | A |
| A-122 | 2,679,915 | G | A |
| A-123 | 2,686,979 | G | A |
| A-124 | 2,693,950 | C | T |
| A-125 | 2,696,737 | C | T |
| A-126 | 2,706,442 | C | T |
| A-127 | 2,709,469 | C | T |
| A-128 | 2,711,214 | C | T |
| A-129 | 2,714,651 | C | T |
| A-130 | 2,721,339 | G | A |
| A-131 | 2,731,030 | G | A |
| A-132 | 2,746,202 | G | A |
| A-133 | 2,805,389 | C | T |
| A-134 | 2,816,733 | G | A |
| A-135 | 2,827,114 | G | A |
| B-1 | 29,724 | G | A |
| B-2 | 92,869 | G | A |
| B-3 | 116,733 | G | A |
| B-4 | 131,184 | G | A |
| B-5 | 156,247 | G | A |
| B-6 | 177,083 | G | A |
| B-7 | 184,379 | G | A |
| B-8 | 212,586 | G | A |
| B-9 | 282,162 | G | A |
| B-10 | 309,483 | G | A |
| B-11 | 376,164 | C | T |
| B-12 | 440,885 | C | T |
| B-13 | 479,120 | G | A |
| B-14 | 722,430 | G | A |
| B-15 | 745,504 | G | A |
| B-16 | 809,993 | G | A |
| B-17 | 859,643 | G | A |
| B-18 | 923,209 | G | A |
| B-19 | 924,973 | G | A |
| B-20 | 998,893 | C | T |
| B-21 | 1,062,144 | C | T |
| B-22 | 1,095,062 | C | T |
| B-23 | 1,102,484 | C | T |
| B-24 | 1,103,812 | C | T |
| B-25 | 1,105,749 | C | T |
| B-26 | 1,107,561 | C | T |
| B-27 | 1,205,722 | C | T |
| B-28 | 1,233,449 | C | T |
| B-29 | 1,242,484 | C | T |
| B-30 | 1,248,388 | C | T |
| B-31 | 1,249,270 | C | T |
| B-32 | 1,291,377 | C | T |
| B-33 | 1,308,597 | C | T |
| B-34 | 1,329,535 | C | T |
| B-35 | 1,367,486 | C | T |
| B-36 | 1,382,065 | C | T |
| B-37 | 1,403,043 | C | T |
| B-38 | 1,433,914 | C | T |
| B-39 | 1,442,447 | C | T |
| B-40 | 1,501,903 | G | A |
| B-41 | 1,504,744 | C | T |
| B-42 | 1,651,403 | G | A |
| B-43 | 1,695,473 | G | A |
| B-44 | 1,779,939 | G | A |
| B-45 | 1,797,452 | G | A |
| B-46 | 1,801,284 | G | A |
| B-47 | 1,816,679 | G | A |
| B-48 | 1,832,252 | G | A |
| B-49 | 1,843,841 | G | A |
| B-50 | 1,868,285 | G | A |
| B-51 | 1,879,922 | G | A |
| B-52 | 1,892,007 | G | A |
| B-53 | 1,916,016 | G | A |
| B-54 | 1,937,604 | G | A |
| B-55 | 1,947,044 | G | A |
| B-56 | 1,948,411 | G | A |
| B-57 | 1,948,649 | G | A |
| B-58 | 1,967,697 | G | A |
| B-59 | 1,974,137 | G | A |
| B-60 | 2,028,284 | C | T |
| B-61 | 2,050,998 | G | A |
| B-62 | 2,052,708 | G | A |
| B-63 | 2,054,372 | G | A |
| B-64 | 2,065,568 | G | A |
| B-65 | 2,067,167 | G | A |
| B-66 | 2,082,577 | G | A |
| B-67 | 2,121,006 | A | G |
| B-68 | 2,149,369 | C | T |
| B-69 | 2,159,680 | G | A |
| B-70 | 2,380,965 | G | A |
| B-71 | 2,477,728 | G | A |
| B-72 | 2,542,800 | G | A |
| B-73 | 2,570,107 | G | A |
| B-74 | 2,647,383 | G | A |
| B-75 | 2,726,248 | C | T |
| B-76 | 2,825,055 | C | T |
| B-77 | 2,837,078 | C | T |
| B-78 | 2,865,322 | C | T |
| B-79 | 2,872,907 | C | T |
| B-80 | 2,880,351 | C | T |
| B-81 | 2,889,394 | C | T |
| B-82 | 2,906,471 | C | T |
| B-83 | 2,927,044 | C | T |
| B-84 | 2,929,963 | C | T |
| B-85 | 2,940,673 | C | T |
| B-86 | 2,946,285 | C | T |
| B-87 | 2,962,909 | C | T |
| B-88 | 2,975,742 | C | T |
| B-89 | 2,987,052 | C | T |
| B-90 | 3,079,560 | C | T |
| B-91 | 3,083,927 | C | T |
| B-92 | 3,090,163 | C | T |

The “specific mutation” may be one or more mutations selected from the mutations shown in Table 1. In other words, the L-glutamic acid-producing bacterium may have one or more mutations selected from the mutations shown in Table 1.

The L-glutamic acid-producing bacterium may have, for example, one or more mutations selected from mutations in group A. The L-glutamic acid-producing bacterium may have, for example, one or more mutations selected from mutations in group B. The L-glutamic acid-producing bacterium may, for example, have one or more mutations selected from mutations in group A and one or more mutations selected from mutations in group B. The L-glutamic acid producing bacterium may, for example, have one or more mutations selected from mutations in group A and may further have one or more mutations selected from mutations in group B. The L-glutamic acid-producing bacterium may, for example, have one or more mutations selected from mutations in group B and may further have one or more mutations selected from mutations in group A. In other words, the “specific mutation” may be, for example, one or more mutations selected from mutations in group A, one or more mutations selected from mutations in group B, or a combination of one or more mutations selected from mutations in group A and one or more mutations selected from mutations in group B.

The number of mutations selected from mutations in group A that the L-glutamic acid-producing bacterium has may be, for example, more 1 or more, 5 or more, 10 or more, 20 or more, 30 or more, 40 or more, 50 or more, 60 or more, 70 or more, 80 or more, 90 or more, 100 or more, 110 or more, 120 or more, or 130 or more, and may be 135 or less, 130 or less, 120 or less, 110 or less, 100 or less, 90 or less, 80 or less, 70 or less, 60 or less, 50 or less, 40 or less, 30 or less, 20 or less, 10 or less, or 5 or less, and may be a combination thereof that is not contradictory. The number of mutations selected from mutations in group A that L-glutamic acid-producing bacterium has may be, for example, 1 to 5, 5 to 10, 10 to 20, 20 to 30, 30 to 40, 40 to 50, 50 to 60, 60 to 70, 70 to 80, 80 to 90, 90 to 100, 100 to 110, 110 to 120, 120 to 130, or 130 to 135. The number of mutations selected from mutations in group A that the L-glutamic acid-producing bacterium has may be, in particular, one or more, 50 or more, 100 or more, 120 or more, 130 or more or 135 or more.

The number of mutations selected from mutations in group B that the L-glutamic acid-producing bacterium has may be, for example, 1 or more, 5 or more, 10 or more, 20 or more, 30 or more, 40 or more, 50 or more, 60 or more, 70 or more, 80 or more, or 90 or more, and may be 92 or less, 90 or less, 80 or less, 70 or less, 60 or less, 50 or less, 40 or less, 30 or less, 20 or less, 10 or less, or 5 or less, or may be a combination thereof that is not contradictory. The number of mutations selected from mutations in group B that the L-glutamic acid-producing bacterium has may specifically be, for example, 1 to 5, 5 to 10, 10 to 20, 20 to 30, 30 to 40, 40 to 50, 50 to 60, 60 to 70, 70 to 80, 80 to 90, or 90 to 92. The number of mutations selected from mutations in group B that the L-glutamic acid-producing bacterium has may be, in particular, 1 or more, 30 or more, 60 or more, 70 or more, 80 or more, or 92.

The L-glutamic acid-producing bacterium may have, for example, 50 or more mutations selected from mutations in group A and 30 or more mutations selected from group B. The L-glutamic acid-producing bacterium may have, for example, 100 or more mutations selected from mutations in group A and 30 or more mutations selected from group B. The L-glutamic acid-producing bacterium may have, for example, 120 or more mutations selected from mutations in group A and 80 or more mutations selected from group B. The L-glutamic acid-producing bacterium may have, for example, 135 mutations in group A and 92 mutations in group B.

The L-glutamic acid-producing bacterium with “specific mutations” may in particular be a coryneform bacterium. The L-glutamic acid-producing bacterium with “specific mutations” may more particularly be a bacterium of the genus Corynebacterium. The L-glutamic acid-producing bacterium with “specific mutations” may more particularly be Corynebacterium casei. The L-glutamic acid-producing bacterium with “specific mutations” may more particularly be a modified strain derived from Corynebacterium casei JCM 12072.

Examples of the L-glutamic acid-producing bacterium with “specific mutations” specifically include Corynebacterium casei RUN5-2-96 (NITE BP-03688) and mutant strains derived therefrom. RUN5-2-96 strain also referred to as “AJ111891”. Examples of the mutant strain derived from RUN5-2-96 include the mutant strain obtained from RUN5-2-96 in the example. The mutant strain obtained from RUN5-2-96 in the example is also referred to as “RUN5-2-96-derived mutant strain in the example” or “AJ111891-derived mutant strain in the example”. RUN5-2-96 is a modified strain derived from Corynebacterium casei JCM 12072 and has all of 135 mutations in group A. RUN5-2-96 was deposited in the National Institute of Technology and Evaluation (NITE NPMD, room 122, 2-5-8 Kazusa-Kamatari, Kisarazu, Chiba, Japan, postal code: 292-0818) on 7 Jul. 2022 as an original international deposit, and given the accession number NITE BP-03688. RUN5-2-96-derived mutant strain in the example is a modified strain derived from Corynebacterium casei JCM 12072, and has all 135 mutations in group A and all 92 mutations in group B. The RUN5-2-96 strain can be obtained, for example, from NITE NPMD.

In Table 1, “genomic position” is the position of each mutation in the nucleotide sequence registered at the National Centre for Biotechnology Information (NCBI; https://www.ncbi.nlm.nih.gov/) with the ACCESSION number of NZ_CP004350.1. Hereinafter, this nucleotide sequence is also referred to as “the sequence of NZ_CP004350.1”. The sequence of NZ_CP004350.1 is the genome sequence of Corynebacterium casei JCM 12072 (LMG S-19264) and is published with annotations. The nucleotide sequence of NZ_CP004350.1 and the annotation of each genomic position can be obtained, for example, from NCBI.

Each mutation shown in Table 1 shall be interpreted as a mutation at a position corresponding to the position of each mutation shown in Table 1 in the genome of each bacterium. For example, mutation A-1 shall be interpreted as a mutation at a position corresponding to position 78,486 of the sequence of NZ_CP004350.1 in the genome of each bacterium. The position of each mutation shown in Table 1 is for convenience in identifying each mutation and does not need to indicate its absolute position in the genome of each bacterium. In other words, the position of each mutation shown in Table 1 is a relative position based on the sequence of NZ_CP004350.1, and the absolute position may vary due to deletion or insertion of nucleic acid residues. For example, in the sequence of NZ_CP004350.1, if one nucleic acid residue is deleted or inserted at a position on the 5′ end side of the sequence rather than position X (X is a positive integer), the original X position becomes position X−1 or X+1, respectively, while the mutation of the original X position A is considered to be a “mutation at a position corresponding to position X in the sequence of NZ_CP004350.1”. The pre-mutation bases shown in Table 1 are for convenience in identifying each mutation and need not be conserved in the bacterial genome before the modification. In other words, if the bacterial genome before the modification does not have the sequence of NZ_CP004350.1, the pre-mutation bases shown in Table 1 may not be conserved. In other words, for each mutation shown in Table 1, “introducing a mutation into the bacterium” means modifying the base at the position of each mutation shown in Table 1 in the bacterial genome before the modification (which is any base other than the post-mutation base) to the post-mutation base shown in Table 1. For example, “introducing mutation A-1 into a bacterium” means modifying the base at the position corresponding to position 78,486 of the sequence of NZ_CP004350.1 in the bacterial genome before modification (which is C, G, or A) to T.

Which position in the genome of each bacterium “corresponds to the position of each mutation shown in Table 1” can be determined by performing an alignment between the genome sequence of each bacterium and the sequence of NZ_CP004350.1. The alignment can be carried out, for example, using known genetic analysis software. Examples of genetic analysis software include DNASIS by Hitachi Solutions and GENETYX by Xenetics (Elizabeth C. Tyler et al., Computers and Biomedical Research, 24(1), 72-96, 1991; Barton G J et al., Journal of molecular biology, 198(2), 327-37. 1987).

For each mutation shown in Table 1, “bacterium having mutation” means that the base at the position of the mutation in the genome of the bacterium is the post-mutation base shown in Table 1, and does not necessarily mean that the bacterium is the one obtained by introducing the mutation. In other words, “bacterium having mutation” for each mutation shown in Table 1 may be bacterium in which the base at the position of the mutation is originally the post-mutation base shown in Table 1, or bacterium obtained by modifying bacterium in which the base at the position of the mutation is not originally the post-mutation base shown in Table 1. For example, “the bacterium having mutation A-1” means that the base at the position of mutation A-1 in the genome of the bacterium (corresponding to position 78,486 in the sequence of NZ_CP004350.1) is T and does not necessarily mean that the bacterium was obtained by introducing mutation A-1. In other words, for example, “bacterium having mutation A-1” may be a bacterium in which the base at the position of mutation A-1 in the genome (corresponding to position 78,486 in the sequence of NZ_CP004350.1) is originally T, or a bacterium obtained by modification in which the base at the position of mutation A-1 in the genome is not originally T. The L-glutamic acid-producing bacterium having a “specific mutation” may in particular be those obtained by the introduction of some or all of the “specific mutations”.

The L-glutamic acid-producing bacterium having a “specific mutation” can be obtained, for example, by introducing a “specific mutation” into a bacterium that do not have a “specific mutation”. The L-glutamic acid-producing bacterium having a “specific mutation” can also be obtained, for example, by introducing the residue of the “specific mutations” into bacterium having part of the “specific mutations”.

The introduction of mutations can be carried out, for example, by known methods. For example, site-directed mutation methods can be used to introduce the desired mutation at the desired position in the genome. Examples of site-specific mutation methods include methods using PCR (Higuchi, R., 61, in PCR technology, Erlich, H. A. Eds., Stockton press (1989); Carter, P., Meth. in Enzymol., 154, 382 (1987)) and phage-based methods (Kramer, W. and Frits, H. J., Meth. in Enzymol., 154, 350 (1987); Kunkel, T. A. et al., Meth. in Enzymol., 154, 367 (1987)).

Each mutation shown in Table 1 may be a mutation that improves the L-glutamic acid-producing ability of the bacterium. Each mutation shown in Table 1 may be a mutation that improves the L-glutamic acid-producing ability of the bacterium, especially when compared to when the position of each mutation is the pre-mutation base shown in Table 1.

Each mutation shown in Table 1 may, for example, be a mutation in a gene (here referring to the coding region of the gene), a mutation in a regulatory region of gene expression such as a promoter, or a mutation in an intergenic region. Whether each mutation shown in Table 1 is a mutation in any region (e.g. gene, gene expression regulatory region or intergenic region) can be confirmed, for example, by referring to the position of the mutation and the annotation in the sequence of NZ_CP004350.1.

If each mutation shown in Table 1 is a mutation in a gene, the mutation may, for example, alter (e.g. increase or decrease) the expression of the gene. If each mutation shown in Table 1 is a mutation in a gene, the mutation may, for example, alter (e.g. increase or decrease) the activity of the protein encoded by the gene.

If each mutation shown in Table 1 is a mutation in a regulatory region of gene expression, the mutation may, for example, alter (e.g. increase or decrease) the expression of the gene.

Altered (e.g. increased or decreased) expression of a gene may, for example, result in altered (e.g. increased or decreased) activity of the protein encoded by the gene.

The L-glutamic acid-producing bacterium with a “specific mutation” may or may not have modifications other than the “specific mutation”, as long as they have an L-glutamic acid-producing ability. The modifications other than the “specific mutation” include known modifications that confer or enhance L-glutamic acid-producing ability. The modifications other than the “specific mutation” include those not selected as the “specific mutation” among the mutations shown in Table 1. The L-glutamic acidproducing bacterium having the “specific mutation” may, for example, has L-glutamic acid-producing ability relying on the “specific mutation”, or may have L-glutamic acid-producing ability relying on a combination of the “specific mutation” and other modifications.

Examples of the L-glutamic acid-producing bacterium specifically include Corynebacterium casei A-013 (NITE BP-03806). The strain A-013 is also referred to as AJ120306. A-013 is a modified strain derived from Corynebacterium casei AJ120306. A-013 was deposited as an original international deposit on 25 Jan. 2023 at the National Institute of Technology and Evaluation (NITE NPMD, room 122, 2-5-8 Kazusa-Kamatari, Kisarazu, Chiba, Japan, postal code: 292-0818). The A-013 strain can be obtained, for example, from NITE NPMD.

The L-glutamic acid-producing bacterium, such as the L-glutamic acid-producing bacterium having the “specific mutation”, may have significantly higher L-glutamic acid-producing ability than, for example, Corynebacterium casei JCM 12072. The term “significantly higher L-glutamic acid-producing ability than Corynebacterium casei JCM 12072” means, for example, the ability to produce and accumulate in the medium L-glutamic acid in an amount at least twice, three times, five times, or seven times greater than that of JCM 12072 when cultured under appropriate culture conditions. The L-glutamic acid-producing bacterium such as L-glutamic acid-producing bacterium having the “specific mutation” may, for example, have L-glutamic acid-producing ability equivalent to or greater than that of Corynebacterium casei RUN5-2-96 strain (NITE BP-03688), mutant strains derived from RUN5-2-96 in Example, or the A-013 strain (NITE BP-03806). The term “L-glutamic acid-producing ability equivalent to or greater than that of Corynebacterium casei RUN5-2-96 strain (NITE BP-03688), mutant strains derived from RUN5-2-96 in Example, or the A-013 strain (NITE BP-03806)” means, for example, the ability to produce and accumulate in the medium L-glutamic acid in an amount of 80% or more, 90% or more, 95% or more, or 100% or more when cultured under appropriate culture conditions. Examples of the appropriate culture conditions include the culture conditions for measuring production amount of L-glutamic acid described in Example 1 (1-2) below (i.e. culture condition in 500 μL of evaluation medium (Table 4) staked in 96 deep-well plates and shaken at 30° C. for 48 h).

L-glutamic acid-producing bacterium such as L-glutamic acid-producing bacterium having the “specific mutation” may harbor a genome sequence which is at least 95%, 96%, 97%, 98%, 99%, 99.5%, 99.7%, 99.9%, 99.95%, 99.97%, or 99.99% identical to, for example, Corynebacterium casei JCM 12072, RUN5-2-96 strain (NITE BP-03688), the mutant strain derived from RUN5-2-96 strain in Example, or A-013 (NITE BP-03806). “Identity” between sequences means the identity between sequences as calculated by blastn using the default setting Scoring Parameters (Match/Mismatch Scores=1, −2; Gap Costs=Linear).

The cell walls of gram-positive bacterium may be commercially available or obtained by manufacturing as appropriate. The method of producing cell walls of gram-positive bacteriumis not restricted.

The cell walls of gram-positive bacterium can be produced, for example, by culturing a gram-positive bacterium in a culture medium. In other words, the method for producing the active ingredient may, for example, comprise a step of culturing the gram-positive bacterium in a culture medium prior to the lysozyme treatment step. The process is also referred to as the “culture step”. The culture step may specifically be a step of culturing the gram-positive bacterium in a culture medium to obtain a culture product.

The medium used is not restricted as long as the gram-positive bacterium can grow. For example, the usual culture medium used for cultivating bacterium such as coryneform bacterium can be used as the culture medium. The medium may contain medium components such as carbon sources, nitrogen sources, phosphate sources, sulphur sources and various other organic and inorganic components as required. The type and concentration of the medium components may be set according to various conditions, such as the type of gram-positive bacterium used. For specific medium compositions, reference may be made, for example, to medium compositions described in previous reports on the production of target substances by bacterium (WO2015/060391, WO2018/030507, WO2015/060391, etc.).

The carbon sources are not restricted as long as they can be capitalised by the gram-positive bacterium. Examples of the carbon sources specifically include, for example, sugars such as glucose, fructose, sucrose, lactose, galactose, xylose, arabinose, waste molasses, starch hydrolysates, and biomass hydrolysates; organic acids such as acetic acid, citric acid, succinic acid, and gluconic acid; alcohols such as ethanol, glycerol, and crude glycerol; and fatty acids. One carbon source may be used, or a combination of two or more carbon sources may be used.

Examples of the nitrogen sources specifically include ammonium salts such as ammonium sulphate, ammonium chloride, and ammonium phosphate; organic nitrogen sources such as peptone, yeast extract, meat extract, plant protein hydrolysate (HVP; e.g. soya protein hydrolysate, soya soy sauce, pea soy sauce); ammonia, and urea. Ammonia gas or ammonia water used for pH adjustment may be used as a nitrogen source. One nitrogen source may be used, or a combination of two or more nitrogen sources may be used.

Examples of the phosphoric acid sources include, specifically, phosphate salts such as potassium dihydrogen phosphate and dipotassium hydrogen phosphate, and phosphate polymers such as pyrophosphoric acid. One phosphate source may be used, or a combination of two or more phosphate sources may be used.

Examples of the sulphur sources specifically include, for example, inorganic sulphur compounds such as sulphate, thiosulphate and sulphite, sulphur-containing amino acids such as cysteine, cystine and glutathione. One sulphur source may be used, or a combination of two or more sulphur sources may be used.

Examples of other organic and inorganic ingredients specifically include an inorganic salt such as sodium chloride, potassium chloride; a trace metal such as iron, manganese, magnesium, calcium; a vitamin such as vitamin B1, vitamin B2, vitamin B6, nicotinic acid, nicotinic acid amide, vitamin B12; an amino acid; a nucleic acid; an organic ingredient such as peptones, casamino acids, yeast extracts, plant protein hydrolysates (HVP; e.g. soya protein hydrolysate, soya soy sauce, pea soy sauce) containing these. Other organic and inorganic ingredients include a defoamer, an osmotic pressure regulator and a osmotic pressure compensating substance for the culture medium. Examples of the defoamer include a silicone-based defoamer (oil, solution, oil compound, emulsion, self-emulsifying, etc.), an alcohol-based defoamer, an oil-based defoamer, a polyether-based defoamer, a vegetable oil (cottonseed oil, linseed oil, soya oil, olive oil, castor oil, coconut oil, etc.). The defoamer can be used in any form, such as liquid, paste, solid, powder, emulsion or wax. Examples of the osmotic compensating substance for the culture medium include a salt such as sodium chloride and potassium chloride, and polysaccharide (such as sorbitol and dextrin), which micro-organisms cannot capitalise on. Examples of the osmotic compensating substance include potassium ion, betaine (glycine betaine), glutamic acid and trehalose. These other organic components may be used as a single component or in combination with two or more components.

Examples of the medium components also include an ingredient of a food product. In other words, the culture medium may contain an ingredient of a food product. Culture of gram-positive bacterium in a medium containing an ingredient of a food product is also referred to as “fermentation of an ingredient of a food product by gram-positive bacterium”. This means that the active ingredient may be, for example, a fermented product of the ingredient of the food product by gram-positive bacterium. The ingredient of the food product may be used singly as a medium component or in combination with other medium components as appropriate.

Examples of the ingredient of the food product used as the medium component (i.e. the ingredient of the food product used to culture gram-positive bacterium) include the ingredients of the food product described below. The ingredient of the food product used as a medium component can be selected independently of the ingredient of the food product used in the method according to the present invention. The ingredient of the food product used as the medium ingredient may or may not be identical to the ingredient of the food product used in the method according to the present invention. The ingredient of the food product used as the medium component may be used singly, or in combination with two or more ingredients.

Examples of the ingredient of the food product used as the medium component specifically include Solanaceae plants. Examples of the Solanaceae plants include a plant of the genus Solanum and a plant of the genus Capsicum. Examples of the plant of the genus Solanum include a tomato and an aubergine. Examples of the plant of the genus Capsicum include green pepper, bell pepper, Shishito pepper and chili pepper. Examples of the green pepper include green-green pepper and red-green pepper. Examples of the plant of the genes Solanum include, in particular, a tomato. The plant of the genus Solanum may be used singly, or in combination with two or more plants.

As the plant of the genus Solanum, for example, fresh (specifically, fresh edible parts such as fruit) may be used for culturing per se or after being subjected to processing as appropriate. Examples of processing include cutting, crushing, backing, pressing, fractionation, dilution, concentration, drying and heating. These processings may be carried out alone or in combination as appropriate. The processing may, for example, remove the skin and/or seeds. Processed product of the Solanaceae plant my be juices, purees and pastes of the Solanaceae plant. That is, for example, processed tomato product may be tomato juice, tomato puree and tomato paste. The term “juice” of the Solanaceae plant, such as tomato juice, may mean, for example, crushed and pressed or backed with edible parts such as fruit, which has a salt-free soluble solids content of less than 8% (w/w) (e.g. 4.5% (w/w) or more and less than 24% (w/w)). A “puree” of the Solanaceae plant, such as tomato puree, may mean, for example, a concentrate of the juice of the Solanaceae plant, which has a salt-free soluble solids content of 8% (w/w) or more and less than 24% (w/w). A “paste” of the Solanaceae plant, such as tomato paste, may mean, for example, a concentrate of the juice of the Solanaceae plant, which has a salt-free soluble solids content of 24% (w/w) or more. The processed product of the Solanaceae plant may or may not contain additives such as sodium chloride. The processed product of the Solanaceae plant may be concentrated and reduced to the salt-free soluble solids content illustrated above.

Culture conditions are not restricted as long as gram-positive bacterium can grow. Culturing can be carried out, for example, under the usual conditions used for culturing bacterium such as coryneform bacterium. The culture condition may be set according to various conditions, such as the type of gram-positive bacterium used. For specific culture conditions, reference can be made, for example, to culture conditions described in previous reports on the production of target substances by bacterium (WO2015/060391, WO2018/030507, WO2015/060391, etc.).

Culturing may be carried out, for example, under aerobic or microaerophilic conditions using a liquid medium. An “aerobic condition” may mean a condition where the concentration of dissolved oxygen in the medium is 0.18 ppm or more, 0.33 ppm ore more, or 1.5 ppm or more. A “microaerophilic condition” may mean a condition where the concentration of dissolved oxygen in the medium is less than 0.33 ppm or less than 0.18 ppm. The concentration of dissolved oxygen in the medium under microaerophilic conditions may be 0.30 ppm or less, 0.25 ppm or less, 0.20 ppm or less, 0.15 ppm or less, 0.10 ppm or less, or 0.05 ppm or less. The concentration of dissolved oxygen can be measured, for example, using sensors such as PL or DO electrodes. Culturing can be carried out, for example, by aeration or shaking culture. The pH of the medium may be, for example, pH 3 to 10 or pH 4.0 to 9.5. During culturing, the pH of the medium can be adjusted if necessary. The pH of the medium can be adjusted using various alkaline or acidic substances such as ammonia gas, ammonia water, sodium carbonate, sodium bicarbonate, potassium carbonate, potassium bicarbonate, magnesium carbonate, sodium hydroxide, potassium hydroxide, calcium hydroxide and magnesium hydroxide. The incubation temperature may be, for example, from 20 to 45° C. or from 25° C. to 37° C. The culturing period may be, for example, from 10 to 120 hours. The culturing may continue, for example, until the carbon source in the medium is consumed or until the gram-positive bacterium are no longer active.

Culturing of gram-positive bacterium under such conditions yields a culture of gram-positive bacterium (specifically, a culture containing gram-positive bacterium).

The bacterial cells may be subjected to lysozyme treatment as contained in the culture (specifically the culture medium) or may be recovered from the culture (specifically the culture medium) and then subjected to lysozyme treatment. In other words, the method of producing the active ingredient may, for example, comprise a step of recovering the bacterial cells from the culture (specifically the culture medium) prior to the lysozyme treatment step. The recovery of the bacterium can, for example, be carried out by known methods. Such methods include spontaneous sedimentation, centrifugation and filtration. The bacterial cells may also be subjected to appropriate treatment before being subjected to lysozyme treatment. In other words, the method of producing the active ingredient may include, for example, a step in which the bacterial cells are subjected to a treatment other than lysozyme treatment prior to the lysozyme treatment process. Examples of treatments other than lysozyme treatment include concentration, drying and heating. Concentration, drying and heating can all be carried out, for example, by known methods. Known drying methods include spray drying and freeze drying. The heating temperature may be, for example, 60° C. or higher, 70° C. or higher, 80° C. or higher, 90° C. or higher, 100° C. or higher, 110° C. or higher or 120° C. or higher. In other words, the cell walls of gram-positive bacterium (specifically, the cells of gram-positive bacterium) subjected to lysozyme treatment may include a culture of gram-positive bacterium, cells recovered from the culture, and their processed products. In other words, the bacterial cells may be provided for lysozyme treatment in the form of, for example, a culture of gram-positive bacterium, bacterial cells recovered from the culture, a treated product thereof, or a combination thereof. In other words, examples of the cell walls of gram-positive bacterium (specifically, the cells of gram-positive bacterium) that are subjected to lysozyme treatment include, for example, the cells contained in the culture of gram-positive bacterium, the cells recovered from the culture, and the cells contained in the treated products thereof. That is, the bacterium may be subjected to lysozyme treatment in the form of, for example, the bacterial cells contained in the culture of the gram-positive bacterium, the bacterial cells recovered from the culture, the bacterial cells contained in the treated products thereof, or a combination thereof. Examples of the treated product include bacterial cells (e.g. bacterial cells contained in the culture or recovered from the culture) subjected to treatment. The cells may be viable, dead or a combination thereof. Cells of gram-positive bacterium contain the cell walls of the gram-positive bacterium.

Fragments of the bacterial cells can also be prepared from the bacterial cells. For example, by crushing the bacterial cells, a bacterial cells-crushed product containing fragments of the bacterial cells can be prepared. In other words, the method of producing the active ingredient may comprise, for example, a step of crushing the cells prior to the lysozyme treatment step. The crushing of the bacterial cells can, for example, be carried out by known methods. Such methods include ultrasonic disruption. The fragments of the bacterial cells may be subjected to lysozyme treatment as contained in the bacterial cells-crushed product, or may be recovered from the bacterial cells-crushed product and then subjected to lysozyme treatment. In other words, the method of producing the active ingredient may, for example, comprise a step of recovering the fragments of the bacterial cells from the bacterial cells-crushed product prior to the lysozyme treatmentstep. The recovery of the fragments of the bacterial cells can be carried out, for example, by known methods. Such methods include spontaneous sedimentation, centrifugation and filtration. The fragments of the bacterial cells may also be subjected to appropriate treatment before being subjected to lysozyme treatment step. In other words, the method for producing the active ingredient may, for example, comprise a step in which the fragments of the bacterial cells are subjected to treatment prior to the lysozyme treatment step. The treatment may include concentration, drying and heating. Concentration, drying and heating can all be carried out, for example, by known methods. Known drying methods include spray drying and freeze drying. The heating temperature may be, for example, 60° C. or higher, 70° C. or higher, 80° C. or higher, 90° C. or higher, 100° C. or higher, 110° C. or higher, or 120° C. or higher. In other words, examples of the cell walls of gram-positive bacterium (specifically, fragments of the cells of gram-positive bacterium) subjected to lysozyme treatment include crushed product of cells of gram-positive bacterium, fragments of bacterial cells recovered from the crushed product and treated product thereof. In other words, the fragments of the bacterial cells may be subjected to lysozyme treatment in the form of, for example, fragments contained in crushed product of cells of gram-positive bacterium, fragments of the bacterial cells recovered from the crushed product, fragments of the bacterial cells contained in the treated product thereof, or a combination thereof. In other words, examples of the cell walls of gram-positive bacterium (specifically, fragments of cells of gram-positive bacterium) that are subjected to lysozyme treatment include fragments of the bacterial cell contained in crushed product of cells of gram-positive bacterium, fragments of the bacterial cell recovered from the crushed product, and fragments of the bacterial cells contained in the treated product thereof. In other words, fragments of bacterium may be subjected to lysozyme treatment in the form of, for example, fragments of bacterial cell contained in crushed product of cells of gram-positive bacterium, fragments of bacterial cells recovered from the crushed product, fragments of bacterial cells contained in the treated product thereof, or a combination thereof. Examples of the treated product includes the fragments of the bacterial cells (e.g. fragments of the bacterial cells contained in the crushed product or fragments of the bacterial cells recovered from the crushed product) submitted to treatment. Fragments of cells of gram-positive bacterium (e.g. crushed product of cells of gram-positive bacterium, fragments of bacterial cells recovered from the crushed product, or treated product thereof) contain the cell wall of gram-positive bacterium.

As mentioned above, the cells and fragments of gram-positive bacterium may be treated by heating or the like. In other words, the cell walls of the gram-positive bacterium subjected to the lysozyme treatment may be treated by heating or the like. In other words, the method of producing the active ingredient may include, for example, a step of subjecting the cell walls of the gram-positive bacterium, such as the cells or fragments thereof, to treatment such as heating prior to the lysozyme treatment step. The cell walls of gram-positive bacterium subjected to lysozyme treatment may in particular be heat treated. The term “subjecting the cell wall of gram-positive bacterium to treatment” includes, unless otherwise stated, the case where the cell walls of gram-positive bacterium in any form is subjected to treatment, in other words, where the cell walls of gram-positive bacterium per se or any fraction containing them are subjected to treatment. For example, “heat treatment of cells of gram-positive bacterium” includes cases where any fraction containing cells of gram-positive bacterium, such as a culture of gram-positive bacterium, bacterial cells recovered from the culture and treated products thereof, is heat treated, unless otherwise stated. The method of producing the active ingredient may also include, for example, a step in which the culture of the gram-positive bacterium is subjected to treatment such as heating prior to the lysozyme treatment step. The term “subjecting the culture to treatment” includes, unless otherwise stated, cases where the culture in any form is subjected to treatment, in other words, where the culture itself or any fraction containing the cell walls of gram-positive bacterium prepared from it is subjected to treatment. By subjecting the culture to heat or other treatment, the cell walls of gram-positive bacterium that have been subjected to heat or other treatment may be obtained.

If the gram-positive bacterium has a target substance-producing ability, the target substance may be produced by the culture of the gram-positive bacterium. In other words, the culture of the gram-positive bacterium may contain the target substance.

The target substance may be recovered from the culture as appropriate. The target substance may be recovered as a appropriate fraction containing the target substance. Examples of such a fraction include a culture supernatant. The target substance may be further separated and purified from such fractions as described above. For example, cells of gram-positive bacterium may be separated from the culture to obtain a culture supernatant, and the target substance may be recovered from the culture supernatant. The “recovery of the target substance” may include the removal of impurities from the fraction containing the target substance. Recovery of the target substance may be carried out, for example, by known methods used for separation and purification of compounds. Examples of such methods include ion exchange resin methods, membrane treatment methods, precipitation, extraction, distillation, crystallisation and activated carbon treatment. The method for recovering the target substance can be selected according to various conditions, for example, the type of target substance. These methods can be used alone or in combination as appropriate. The recovered target substance may contain, in addition to the target substance, other components such as, for example, cells of gram-positive bacterium, medium components, water and metabolic by-products of gram-positive bacterium. The purity of the recovered target substance may be, for example, 30% (w/w) or more, 50% (w/w) or more, 70% (w/w) or more, 80% (w/w) or more, 90% (w/w) or more, or 95% (w/w) or more.

The use of the recovered target material is not restricted.

The recovered target substance may be used separately from the active ingredient or in combination with the active ingredient. The target substance may, for example, be prepared as a fermented seasoning containing the target substance and used in combination with the active ingredient.

The recovered target substance may also be combined with the cell walls of gram-positive bacterium and subjected to lysozyme treatment. The target substance may, for example, be prepared as a fermented seasoning containing the target substance and combined with the cell walls of gram-positive bacterium and subjected to lysozyme treatment.

Alternatively, a fraction containing both the target substance and the cell walls of gram-positive bacterium may be subjected to lysozyme treatment. For example, a culture containing both the target substance and the cell walls of gram-positive bacterium may be subjected to lysozyme treatment as it is, or dried or otherwise treated as appropriate.

The conditions of lysozyme treatment are not restricted as long as a treated product with a flavor-improving function is obtained. The conditions of lysozyme treatment may, for example, be set so that a treated product with a higher flavor-improving function compared to the cell walls of gram-positive bacterium is obtained.

“Lysozyme” may refer to a protein (EC 3.2.1.17) that has the activity to catalyse a reaction that hydrolyses peptidoglycan. The same activity is also referred to as “lysozyme activity”.

The origin of lysozyme is not restricted. The lysozyme may be derived from any source, such as micro-organisms, animals or plants. The lysozymes may be, in particular, a lysozyme derived from egg white. Examples of egg white include egg white from chicken eggs. Known homologue of lysozyme may also be used as the lysozyme. Artificial variant of known lysozymes or homologues thereof may also be utilised as lysozymes. Such homologues or artificial variants are not restricted as long as they have lysozyme activity. Lysozymes may, for example, be obtained by heterologous expression (i.e. recombinant enzymes). The lysozyme may, for example, be commercially available or may be obtained by appropriate manufacturing. Examples of the commercially available lysozyme include Lysozyme BIO (from egg white, manufactured by BIOCON (JAPAN) LTD.).

Lysozyme can be produced, for example, by separation (e.g. extraction) from lysozyme-containing agricultural, fisheries and livestock products (e.g. egg white). Lysozyme can be produced, for example, by culturing lysozyme-producing host cells. Lysozyme-producing host cells may be lysozyme-producing by nature or may be modified to produce lysozyme. Lysozyme-producing host cells can be obtained, for example, by expressively introducing a gene encoding lysozyme into the host cell. The culture conditions of the lysozyme-producing host cells are not restricted as long as lysozyme is produced. Lysozyme-producing host cells can, for example, be cultured under the usual conditions for culturing host cells.

Lysozymes may or may not contain components other than lysozymes. In other words, purified lysozyme may be used as lysozyme, or lysozyme-containing material may be used. The lysozyme may be purified to the desired degree.

One type of lysozyme may be used, or a combination of two or more lysozymes may be used.

Lysozyme treatment can be carried out by bringing lysozyme into contact with the cell walls of gram-positive bacterium. Subjecting the cell walls of gram-positive bacterium to lysozyme treatment is also referred to as “acting on the cell walls of gram-positive bacterium with lysozyme”. Lysozyme treatment may be carried out, for example, in a liquid. The liquid may be an aqueous medium such as water or aqueous buffer. For example, a suspension containing the cell walls of gram-positive bacterium may be prepared and lysozyme added to it. For example, a treatment solution containing lysozyme may be prepared and the cell walls of gram-positive bacterium may be added to it. For example, a suspension containing the cell walls of gram-positive bacterium and a treatment solution containing lysozyme may also be prepared and mixed.

Lysozyme treatment may or may not be carried out under heat. The temperature of the lysozyme treatment may be, for example, the temperature at which the lysozyme is active. The temperature of the lysozyme treatment may be, for example, 10° C. or more, 20° C. or more, 30° C. or more, 40° C. or more, or 50° C. or more, 60° C. or less, 50° C. or less, 40° C. or less, 30° C. or less, or 20° C. or less, or any consistent combination thereof. The temperature of the lysozyme treatment may specifically be, for example, from 10 to 20° C., from 20 to 30° C., from 30 to 40° C., from 40 to 50° C., or from 50 to 60° C. The temperature of the lysozyme treatment may specifically be, for example, from 20 to 60° C., from 30 to 60° C., or from 40 to 60° C. The pH of the lysozyme treatment may be, for example, the pH at which the lysozyme is active. The pH of the lysozyme treatment may be, for example, from 3 to 9. The duration of the lysozyme treatment may be, for example, the time for which the flavor-improving function is improved to the desired degree. The duration of the lysozyme treatment may be, for example, 0.5 hours or more, 1 hour or more, 2 hours or more, 4 hours or more, 6 hours or more, 8 hours or more, 10 hours or more, 12 hours or more, 15 hours or more or 18 hours or more, 24 hours or less, 18 hours or less, 15 hours or less, 12 hours or less, 10 hours or less, 8 hours or less, 6 hours or less, 4 hours or less, 2 hours or less, or 1 hour or less, or any non-contradictory combination thereof. The duration of the lysozyme treatment may specifically be, for example, from 0.5 to 1 hour, from 1 to 2 hours, from 2 to 4 hours, from 4 to 6 hours, from 6 to 8 hours, from 8 to 10 hours, from 10 to 12 hours, from 12 to 15 hours, from 15 to 18 hours, or from 18 to 24 hours. The duration of lysozyme treatment may specifically be, for example, from 0.5 to 24 hours, from 1 to 18 hours or from 2 to 12 hours.

The amount of lysozyme used may be, for example, 0.00005 g or more, 0.0001 g or more, 0.0002 g or more, 0.0005 g or more, 0.001 g or more, 0.002 g or more, 0.005 g or more, 0.01 g or more, 0.02 g or more, 0.05 g or more, 0.1 g or more, 0.2 g or more, or 0.5 g or more, may be 1 g or less, 0.5 g or less, 0.2 g or less, 0.1 g or less, 0.05 g or less, 0.02 g or less, 0.01 g or less or less, 0.005 g or less, 0.002 g or less, 0.001 g or less, 0.0005 g or less, 0.0002 g or less, or 0.0001 g or less, or any non-contradictory combination thereof, with respect to 1 g of dried cells of gram-positive bacterium. The amount of lysozyme used may specifically be, for example, from 0.0005 to 0.0001 g, from 0.0001 to 0.0002 g, from 0.0002 to 0.0005 g, from 0.0005 to 0.001 g, from 0.001 to 0.002 g, from 0.002 to 0.005 g, from 0.005 to 0.01 g, from 0.01 to 0.02 g, from 0.02 to 0.05 g, from 0.05 to 0.1 g, from 0.1 to 0.2 g, from 0.2 to 0.5 g or from 0.5 to 1 g, with respect to 1 g of dried cells of gram-positive bacterium. The amount of lysozyme used may specifically be, for example, from 0.00005 to 1 g, from 0.00005 to 0.2 g, from 0.0001 to 0.1 g, or from 0.0002 to 0.05 g for 1 g of dried gram-positive bacterium, with respect to 1 g of dried cells of gram-positive bacterium.

The amount of lysozyme used is, for example, 1,950 FIP units or more, 3,900 FIP units or more, 7,800 FIP units or more, 19,500 FIP units or more, 39,000 FIP units or more, 78,000 FIP units or more, 195,000 FIP units or more, 390,000 FIP units or more, 780,000 FIP units or more, 1,950,000 FIP units or more, 3,900,000 FIP units or more, 7,800,000 FIP units or more or 19,500,000 FIP units or more. May be 39,000,000 FIP units or less, 19,500,000 FIP units or less, 7,800,000 FIP units or less, 3,900,000 FIP units or less, 1,950,000 FIP units or less, 780,000 FIP units or less, 390,000 FIP units or less, 195,000 FIP units or less, 78,000 FIP units or less, 39,000 FIP units or less, 19,500 FIP units or less, 7,800 FIP units or less, or 3,900 FIP units or less, or any non-conflicting combination thereof. It may also be. The amount of lysozyme used may be, for example, 1,950-3,900 FIP units, 3,900-7,800 FIP units, 7,800-19,500 FIP units, 19,500-39,000 FIP units, 39,000-78,000 000 FIP units, 78,000 to 195,000 FIP units, 195,000 to 390,000 FIP units, 390,000 to 780,000 FIP units, 780,000 to 1,950,000 FIP units, 1,950,000 to 3,900,000 FIP units 3,900,000 to 7,800,000 FIP units, 7,800,000 to 19,500,000 FIP units or 19,500,000 to 39,000,000 FIP units. The amount of lysozyme used may specifically be, for example, 1,950-39,000,000 FIP units, 1,950-7,800,000 FIP units, 3,900-3,900,000 FIP units, or 7,800-1,950,000 FIP units.

If the cell walls of gram-positive bacterium are subjected to lysozyme treatment in a form other than the bacterial cells (e.g. in the form of fragments of the bacterial cells), the dry weight of the cell walls of gram-positive bacterium may be converted to the equivalent to dry weight of the cells of the gram-positive bacterium. In other words, “to 1 g of dried cells of gram-positive bacterium” in the description of the amount of lysozyme used may be read, for example, as “to 1 g of cell walls prepared from cells of gram-positive bacterium in terms of dry weight”.

The activity of lysozyme can be determined by the following procedure. Namely, one FIP unit is defined as the amount of enzyme that reduces the absorbance at 450 nm by 0.001 per minute at pH 7.0 and 25° C. when an enzyme reaction is carried out using a suspension of Micrococcus lysodeikticus organisms as substrate (Shugar D, Biochimica et Biophysica Acta, Vol. 8, 302-309 (1952)).

The lysozyme-treated product may be used as an active ingredient either as it is or after being subjected to appropriate treatment. In other words, the method of producing the active ingredient may, for example, comprise a step in which the lysozyme-treated product is subjected to additional treatment. Examples of additional treatments include concentration, drying, heating and fractionation. All of these treatments can be carried out, for example, by known methods. Known drying methods include spray drying and freeze drying. Heating may be carried out, for example, to inactivate the lysozyme. The heating temperature may be, for example, 60° C. or higher, 70° C. or higher, 80° C. or higher, 90° C. or higher, 100° C. or higher, 110° C. or higher or 120° C. or higher. The heating temperature may be, for example, from 1 to 30 minutes or from 5 to 20 minutes. “Fractionation” may also encompass the separation (e.g. purification) of specific components. Fractionation may be carried out so that fractions or components with a flavor-improving function are obtained. The fractions or components with a flavor-improving function may be a supernatant of the lysozyme-treated product, medium and low molecular weight fractions of the lysozyme-treated product and components contained therein. The supernatant can be obtained, for example, by centrifugation or filtration. Filtration can be carried out, for example, using microfilters (e.g. membranes with a pore size of 0.1 to 0.5 μm), filter paper or filter cloth. The term “medium and low molecular weight fractions” may refer to fractions obtained as permeate by filtration of the object (e.g. supernatant) through an ultrafiltration membrane with a nominal fractional molecular weight of 30000 or less, 10000 or less, 5000 or less, 3000 or less, or 1000 or less. Examples of components contained in the supernatant or medium/low molecular weight fraction of the lysozyme-treated product include a constituent characteristic of peptidoglycans. Examples of a constituent characteristic of peptidoglycans include N-acetylglucosamine, N-acetylmuramic acid, diaminopimeric acid, D-isoglutamine, D-alanine, one or compounds containing one or more of them as a constituent. In other words, the active ingredients include lysozyme-treated products themselves or those that have been subjected to additional treatment.

The active ingredient may, for example, be used in combination with other components (i.e. components other than the active ingredient). Other components may include a target substance and a culture of gram-positive bacterium. By combining the active ingredient with other components, the flavor-improving effect may be enhanced compared to when the active ingredient is used alone. Specifically, for example, the flavor-improving effect may be enhanced when the active ingredient is used in combination with L-glutamic acid and/or a culture of gram-positive bacterium, compared to when the active ingredient is used alone. Also, for example, when the active ingredient is used in combination with L-glutamic acid, the umami flavor of the food product may be enhanced compared to when the active ingredient is used alone. The term “active ingredient in combination with L-glutamic acid”, unless otherwise stated, includes cases where the active ingredient is used in combination with L-glutamic acid in any form, in other words, where the active ingredient is used in combination with L-glutamic acid itself or any fraction containing it (e.g. a culture containing L-glutamic acid).

The target substance is described above. The target substance may be a commercially available product or one that has been manufactured and obtained as appropriate. The target substance can be produced, for example, by chemical synthesis, enzyme reaction, fermentation method, extraction method or a combination thereof. The target substance can specifically be produced, for example, by fermentation methods using a micro-organism having the target substance-producing ability. The micro-organism having the target substance-producing ability may or may not be the same as the gram-positive bacterium from which the active ingredient is derived. The target substance may, for example, be produced along with the production of the active ingredient or separately from the active ingredient. The target substance may or may not be purified to the desired degree. In other words, the target substance may be a purified product or a material containing the target substance. The term “material containing the target substance” means a material containing 0.1% (w/w) or more of the target substance. Specifically, examples of the material containing the target substance include, for example, a fermented product such as a culture, cells and a culture supernatant obtained by culturing the micro-organism having target substance-producing ability, and their processed products. Examples of the processed products include a material such as the above-mentioned fermented product that has been subjected to treatment such as heating, concentration, dilution, drying and fractionation. The content of the target substance in the material containing the target substance may be, for example, 1% (w/w) or more, 3% (w/w) or more, 5% (w/w) or more, 10% (w/w) or more, 30% (w/w) or more, 50% (w/w) or more, 70% (w/w) or more, 90% (w/w) or more or 95% (w/w) or more. The term “using the active ingredient and the target substance in combination” is not limited to the case where the active ingredient and the target substance obtained individually are used in combination, but also includes the case where the active ingredient and the target substance obtained collectively are used together. The case of using the active ingredient and the target substance obtained collectively includes the case where a culture containing both the target substance and the gram-positive bacterium is subjected to lysozyme treatment to prepare the active ingredient and utilize them. In other words, the target substance may be subjected to lysozyme treatment.

A culture of gram-positive bacterium as other component (i.e. component other than the active ingredient) can be obtained by culturing gram-positive bacterium. The culture of gram-positive bacterium is described above. The gram-positive bacterium from which the culture as the other component is derived may or may not be the same as the gram-positive bacterium from which the active ingredient is derived. The culture as the other component may, for example, have been produced along with the production of the active ingredient, or may have been produced separately from the active ingredient. The culture as other component may, for example, be used as additional component either as they are or after being subjected to fractionation (e.g. separation of bacterial cells or the target substance), concentration, drying, heating or other treatment. By fractionation, for example, 90% or more, 95% or more, 97% or more, or 99% or more of the total number of bacterium may be isolated (i.e. removed from the culture). The culture as the other component may or may not contain cells of the gram-positive bacterium. The culture as the other component may or may not contain the target substance (e.g. L-glutamic acid). The term “using the active ingredient and the culture of gram-positive bacterium in combination” is not limited to the case where the active ingredient and the culture obtained individually are used in combination, but also includes the case where the active ingredient and the culture obtained collectively are used. The case of using the active ingredient and the culture obtained collectively includes the case where the culture containing the gram-positive bacterium is subjected to lysozyme treatment to prepare the active ingredient and utilize them. In other words, the culture material as the other component may be subjected to lysozyme treatment.

### <2> Composition of the Invention

The composition of the present invention is a composition containing an active ingredient.

In other words, the composition of the present invention is a composition containing the following component (A):
(A) Lysozyme-treated product of cell walls of gram-positive bacterium.

The composition of the present invention may be used to improve the flavor of food product, i.e. the flavor-improving effect is achieved. Accordingly, the composition of the present invention may be used to improve the flavor of food product. In other words, the composition of the present invention may be, for example, a composition for improving the flavor of a food product. The flavor improvement may be, for example, an enhancement of spiciness and/or an impartment of kokumi. The flavor improvement may be, in particular, an enhancement of the pungency of the spice and/or the imparting of a thickness.

The composition of the present invention may also be used to produce food products with improved flavor. Thus, the composition of the present invention may be utilised in the manufacture of food products (specifically, in the manufacture of food products with improved flavor). In other words, the composition of the present invention may, for example, be a composition for the manufacture of food products (specifically, the manufacture of food products with improved flavor).

The composition of the present invention may be, for example, a seasoning. The composition of the present invention may specifically be, for example, a seasoning for improving the flavor of a food product, or a seasoning for the production of a food product (specifically, the production of a food product with improved flavor).

The composition of the present invention may or may not itself have an improved flavor. For example, if the composition of the present invention contains a spice, the composition of the present invention may itself have an enhanced spice taste (specifically, an enhanced spice taste compared to the composition without the active ingredient).

The compositions of the invention may be used to improve flavor or to produce food products in the manner described in the methods of the invention below.

The composition of the present invention may consist of the active ingredient or may contain other component than the active ingredient. In one aspect, the composition of the present invention may exclude a composition comprising an active ingredient. The component other than the active ingredient may comprise one component or a combination of two or more components.

The active ingredient contained in the composition of the present invention may be produced by the method of producing the active ingredient described above.

In other words, the present specification discloses a method of producing the composition of the present invention, comprising a step of producing an active ingredient by the method of producing an active ingredient described above. The step of producing the active ingredient in the method of producing the composition of the present invention may specifically be a step of obtaining the active ingredient by subjecting the cell walls of gram-positive bacterium to lysozyme treatment.

The component other than the active ingredient are not restricted as long as the flavor-improving effect is not lost (i.e. the flavor-improving effect of the active ingredient is obtained). The component other than the active ingredient can be selected according to various conditions, such as the type of food product, for example. Examples of the component other than the active ingredient include components that are used in foods or medicines.

The component other than active ingredient may be, specifically, a component that is effective in the manufacture of food products. Examples of the component effective in the production of food products include the ingredient of the food product described below. Examples of the component other than the active ingredient include, in particular, a culture of gram-positive bacterium, a target substance and a spice. In other words, for example, the composition of the present invention may be a composition containing an active ingredient and a culture of gram-positive bacterium (e.g. seasoning), a composition containing an active ingredient and a target substance (e.g. seasoning), a composition containing an active ingredient and a spice (e.g. seasoning), a composition containing an active ingredient, a culture of gram-positive bacterium and a spice (e.g. seasoning), a composition containing an active ingredient, a target substance and a spice (e.g. seasoning), or a composition containing an active ingredient, a culture of gram-positive bacterium, a target substance and a spice (e.g. seasoning).

The target substance is described above. The term “the composition contains the active ingredient and the target substance” is not limited to cases where the active ingredient and the target substance obtained individually are contained in the composition, but also includes cases where the active ingredient and the target substance obtained collectively are contained in the composition. A case in which a collectively obtained active ingredient and a target substance are contained in a composition is when a culture containing both the target substance and the gram-positive bacterium is subjected to lysozyme treatment to prepare the active ingredient, which is then contained in the composition.

The culture of gram-positive bacterium as the component other than the active ingredient are described above. The term “a composition contains an active ingredient and a culture of gram-positive bacterium” is not limited to cases where the active ingredient and the culture are individually obtained and contained in the composition, but also includes cases where the active ingredient and the culture are obtained collectively and contained in the composition. A case in which the active ingredient and the culture obtained collectively are included in the composition is when a culture containing cells of the gram-positive bacterium is subjected to lysozyme treatment to prepare the active ingredient, which is then included in the composition.

“Spice” may mean any leaf, stem, bark, root, flower, bud, seed, fruit or rind of a plant used for the purpose of imparting a special flavor to a food product. “Spices” may also encompass so-called “herbs”. Examples of spices include a spice of the family Lauraceae (e.g., bay leaf, cinnamon), a spice of the family Piperaceae (e.g., black pepper, white pepper), a spice of the family Lamiaceae (e.g., thyme, sage, basil, oregano, marjoram, rosemary, mint), a spice of the family Apiaceae (e.g., fennel, caraway, cumin, coriander, parsley, Italian parsley, celery, celery seed, dill), a spice of the family Solanaceae (e.g., chili pepper, cayenne pepper), a spice of the family Myristicaceae (e.g., nutmeg, mace), a spice of the family Alliaceae (e.g., garlic powder, onion powder), a spice of the family Myrtaceae (e.g., allspice, clove), a spice of the family Schisandraceae (e.g., star anise), a spice of the family Fabaceae (e.g., fenugreek), a spice of the family Polygonaceae (e.g., knotweed), a spice of the family Brassicaceae (e.g., garden cress, wasabi, Japanese mustard), a spice of the family Zingiberaceae (e.g., cardamom, turmeric, ginger), and a spice of the family Rutaceae (e.g., sansho (Japanese pepper)). Particularly, examples of the spice include a spice of the family Piperaceae, a spice of the family Solanaceae, a spice of the family Brassicaceae, and a spice of the family Rutaceae, which have a pungent or spicy flavor. In accordance with the general practice, garlic and onions, which are used for cooking in their raw state as fresh vegetables, may be excluded from the spices in the present invention. In the present invention, one spice may be used, or a combination of two or more spices may be used.

The composition of the present invention may be produced, for example, by combining (e.g. mixing) the active ingredient and optionally other components as appropriate. Some or all of the other components may be mixed with the cell walls of the gram-positive bacterium prior to lysozyme treatment and subjected to lysozyme treatment. In other words, some or all of the other components may be subjected to lysozyme treatment.

The composition of the present invention may, for example, be formulated as appropriate. In the formulation, additives may be used as appropriate. Examples of additives include an excipient, a binder, a disintegrant, a lubricant, a stabiliser, a taste and odour correcting agent, a diluent, a surfactant and a solvent. The additives may be selected according to various conditions, for example, the form of the composition of the present invention.

The form of the composition of the present invention is, in particular, not restricted. The composition of the present invention may be in any form, for example, powders, flakes, tablets, pastes, liquids, etc.

The content and content ratio of each component (i.e. the active ingredient and optionally other components) in the composition of the present invention is not restricted as long as the flavor-improving effect is achieved. The content and content ratio of each ingredient in the composition of the present invention can be set according to various conditions, such as the mode of use of the composition of the present invention.

The content of the active ingredient in the composition of the present invention is more than 0% (w/w) and 100% (w/w) or less. The content of the active ingredient in the composition of the present invention is, for example, 0.1% (w/w) or more, 0.2% (w/w) or more, 0.5% (w/w) or more, 1% (w/w) or more, 2% (w/w) or more, 5% (w/w) or more, 10% (w/w) or more, 20% (w/w) or more, 30% (w/w) or more, 50% (w/w) or more, 70% (w/w) or more, may be 100% (w/w) or less, less than 100% (w/w), 99.9% (w/w) or less, 90% (w/w) or less, 70% (w/w) or less, 50% (w/w) or less, 30% (w/w) or less, 20% (w/w) or less, 10% (w/w) or less, 5% (w/w) or less, 2% (w/w) or less, or 1% (w/w) or less, or may be any combination thereof that is not contradictory. The content of the active ingredient in the composition of the present invention may be, for example, from 0.1% (w/w) to 1% (w/w), from 1% (w/w) to 10% (w/w), from 10% (w/w) to 20% (w/w), from 20% (w/w) to 30% (w/w), from 30% (w/w) to 50% (w/w), from 50% (w/w) to 7 0% (w/w), or from 70% (w/w) to 100% (w/w). The content of the active ingredient in the composition of the present invention may specifically be, for example, from 0.1 to 100% (w/w), from 0.2 to 70% (w/w), or from 0.5 to 50% (w/w).

The content of the active ingredient in the composition of the present invention may, for example, be 0.1% (w/w) or more, 0.2% (w/w) or more, 0.5% (w/w) or more, 1% (w/w) or more, 2% (w/w) or more, 5% (w/w) or more, 10% (w/w) or more, 20% (w/w) or more, 50% (w/w) or more, 100% (w/w) or more, 200% (w/w) or more, 500% (w/w) or more, 1000% (w/w) or more, 2000% (w/w) or more, or 5000% (w/w) or more, based on the dry weight of the cells of the original gram-positive bacterium. It may also be 10000% (w/w) or less, 5000% (w/w) or less, 2000% (w/w) or less, 1000% (w/w) or less, 500% (w/w) or less, 200% (w/w) or less, 100% (w/w) or less, 50% (w/w) or less, 20% (w/w) or less, 10% (w/w) or less, 5% (w/w) or less, 2% (w/w) or less, 1% (w/w) or less, 0.5% (w/w) or less, or 0.2% (w/w) or less. It may include any combination of these ranges that does not contradict. Specifically, the content of the active ingredient in the composition of the present invention may, for example, be in the range of 0.1 to 0.2% (w/w), 0.2 to 0.5% (w/w), 0.5 to 1% (w/w), 1 to 2% (w/w), 2 to 5% (w/w), 5 to 10% (w/w), 10 to 20% (w/w), 20 to 50% (w/w), 50 to 100% (w/w), 100 to 200% (w/w), 200 to 500% (w/w), 500 to 1000% (w/w), 1000 to 2000% (w/w), 2000 to 5000% (w/w), or 5000 to 10000% (w/w), based on the dry weight of the dry weight of the cells of the original gram-positive bacterium. Specifically, it may also be in the ranges of 0.1 to 10000% (w/w), 0.2 to 5000% (w/w), or 0.5 to 2000% (w/w), based on the dry weight of the cells of the original gram-positive bacterium. The term “dry weight of the cells of the original gram-positive bacterium” means the dry weight of the cells of the gram-positive bacterium from which the active ingredient is derived. The dry weight of the bacterial cells is also referred to as “Dry Cell Weight (DCW)”. If the cell walls of the gram-positive bacterium is subjected to lysozyme treatment in a form other than the bacterial cells (e.g. in the form of fragments of the bacterial cells), the dry weight of the cell walls of gram-positive bacterium may be converted to the equivalent dry weight of the cells of the gram-positive bacterium. For example, if 100 parts by weight of the composition of the present invention contain an active ingredient obtained from 1 part by weight (dry weight) of the cells of gram-positive bacterium, the content of the active ingredient in the composition of the present invention is 1% (w/w) in terms of the dry weight of the original gram-positive bacterium. A content exceeding 100% (w/w) in terms of dry weight of the cells of the original gram-positive bacterium means that the active ingredient is concentrated and contained in the composition of the present invention. In other words, for example, a content of 200% (w/w) means that the active ingredient is twice as concentrated and contained in the composition of the present invention. For example, if the supernatant of a lysozyme-treated product obtained from 200 parts by dry weight of the cells of gram-positive bacterium is concentrated and contained as the active ingredient in 100 parts by weight of the composition of the present invention, the content of the active ingredient in the composition of the present invention is 200% (w/w) in terms of the dry weight of the cells of the original gram-positive bacterium.

The composition of the present invention may, for example, fulfil one or more (e.g. one, two, three or all four) conditions selected from the following conditions (1) to (4). That is, the content of the active ingredient in the composition of the present invention may be, for example, an amount in which the composition of the present invention satisfies one or more (e.g. one, two, three or all four) of the conditions selected from conditions (1) to (4) below. Conditions (1) to (4) below may all apply, for example, when the composition of the present invention is present in a form containing a liquid fraction such as a liquid or paste or when the composition of the invention is dispersed (e.g. dissolved or suspended) in an aqueous medium such as water or an aqueous buffer solution.

Condition (1): the content of constituent characteristic of peptidoglycans in the clear fraction of the composition of the present invention (especially the medium and low molecular weight fractions) is in the predetermined range.

The term “clear fraction” may refer to the liquid fraction obtained by subjecting the composition of the present invention to centrifugation or filtration. The term “medium and low molecular weight fractions” may mean fractions obtained as permeate by filtering the object (e.g. the clear fraction) through an ultrafiltration membrane with a nominal fraction molecular weight of 30000 or less, 10000 or less, 5000 or less, 3000 or less, or 1000 or less. Examples of the constituent characteristic of peptidoglycans include N-acetylglucosamine, N-acetylmuramic acid, diaminopimeric acid, D-isoglutamine and D-alanine. The predetermined range in condition (1) may be, for example, for each of the components independently, a molar ratio of 0.025 or more, preferably 0.05 or more, more preferably 0.1 or more, more preferably 0.1 or more, more preferably 0.15 or more, more preferably 0.2 or more, more preferably 0.25 or more, as a ratio of the content of the component in the clear fraction of the composition of the present invention (especially the medium and low molecular weight fraction) to the content of the component in the composition of the present invention. The content of each component in condition (1) means the total content of the same component present in any form (e.g. as a single molecule or in a compound with other components, etc.). For example, the content of N-acetylglucosamine in the composition of the present invention in condition (1) means the total content of N-acetylglucosamine present in any form (e.g. present as a single molecule, or constituting a peptidoglycan, etc.). The total content of each component, including the peptidoglycan-constituting form, can be measured after the peptidoglycan has been degraded by hydrolysis or other treatments using acids or alkalis. When measuring the total content of amino acids, including the forms constituting the peptidoglycan, for example, a heat reaction is carried out at 120° C. for 24 hours in 6 mol hydrochloric acid under reduced pressure. When analysing the total content of amino sugars, including the forms comprising peptidoglycan, for example, a heating reaction is carried out at 105° C. for 12 hours in 4 molar hydrochloric acid under reduced pressure. In this case, N-acetylglucosamine is converted to glucosamine and N-acetylmuraminic acid to muramic acid, so the content can be measured as the content of the respective converted substances (glucosamine and muramic acid). The content of each constituent in the peptidoglycan-constituting form can also be determined by measuring the content of each constituent under conditions in which the peptidoglycan is not hydrolysed (amount in the free form), and subtracting it from the content of each constituent under conditions in which the peptidoglycan is hydrolysed (total amount in the free and peptidoglycan constituting forms). The predetermined range in condition (1) for the content of diaminopimelic acid may be, for example, as the content of diaminopimeric acid of the bound type (in the form comprising peptidoglycan) contained in the fraction with fractional molecular weight of 10,000 Da or less with respect to 100 g of the dry weight of the composition of the present invention (if the composition of the present invention contains an excipient, the weight of the excipient is excluded), 20.0 mg or more, preferably 25.0 mg or more, even more preferably 30.0 mg or more, even more preferably 35.0 mg or more, even more preferably 37.5 mg, even more preferably 40.0 mg or more.

Condition (2): the ratio of the content of reducing sugar ends to the content of total sugar in the clear fraction of the composition of the present invention (especially the medium and low molecular weight fraction) is in the predetermined range.

The predetermined range in condition (2) may be, for example, a molar ratio of 0.001 or more, preferably 0.005 or more, more preferably 0.01 or more, more preferably 0.015 or more, more preferably 0.02 or more.

Condition (3): the clear fraction of the composition of the present invention (especially the medium and low molecular weight fraction) has a function to enhance the response of TRPV1 receptor to pungent components (e.g. capsaicin).

The clear fraction of the composition of the present invention (especially the medium-low molecular weight fraction) may, for example, have a function to enhance the response of TRPV1 receptor to pungent components (e.g. capsaicin) by 1.1-fold or more, 1.3-fold or more, 1.5-fold or more, or 2-fold or more.

Condition (4): the molecular weight of the peptidoglycan-derived glycopeptides or glycan chains contained in the clear fraction of the composition of the present invention (especially the medium and low molecular weight fraction), or a value reflecting it, is below a predetermined range. The molecular weight can be determined, for example, by mass spectrometry, gel filtration chromatography, gel exclusion chromatography or light scattering. Examples of the numerical value reflecting the molecular weight include the diffusion coefficient determined by two-dimensional NMR.

If the composition of the present invention contains a spice, the content of the spice in the composition of the present invention may be, for example, 0.1% (w/w) or more, 0.2% (w/w) or more, 0.5% (w/w) or more, 1% (w/w) or more, 2% (w/w) or more, 5% (w/w) or more, 10% (w/w) or more, 20% (w/w) or more, 30% (w/w) or more, 50% (w/w) or more, or 70% (w/w) or more, may be 90% (w/w) or less, 70% (w/w) or less, 50% (w/w) or less, 30% (w/w) or less, 20% (w/w) or less, 10% (w/w) or less, 5% (w/w) or less, 2% (w/w) or less, or 1% (w/w) or less, or any non-contradictory combination thereof. The content of the spice in the composition of the present invention may be, for example, 0.1% (w/w) to 1% (w/w), 1% (w/w) to 10% (w/w), 10% (w/w) to 20% (w/w), 20% (w/w) to 30% (w/w), 30% (w/w) to 50% (w/w), 50% (w/w) to 7 0% (w/w), or 70% (w/w) to 90% (w/w). The content of the spice in the composition of the present invention may specifically be, for example, 0.2 to 90% (w/w), 0.5 to 50% (w/w) or 1 to 20% (w/w).

If the composition of the present invention contains a spice, the content of spice in the composition of the present invention may be, for example, with respect to 1 part by weight of the active ingredient contained in the composition of the present invention (calculated as the dry weight of the original cells of gram-positive bacterium), 0.2 parts by weight or more, 0.5 parts by weight or more, 1 part by weight or more, 2 parts by weight or more, 5 parts by weight or more, 10 parts by weight or more, 20 parts by weight or more, 50 parts by weight or more, or 100 parts by weight or more, may be 1000 parts by weight or less, 500 parts by weight or less, 200 parts by weight or less, 100 parts by weight or less, 50 parts by weight or less, 20 parts by weight or less, 10 parts by weight or less, 5 parts by weight or less, or 2 parts by weight or less, or may be a non-contradictory combination thereof. The content of spice in the composition of the present invention may be, for example, with respect to 1 part by weight of the active ingredient contained in the composition of the present invention (calculated as the dry weight of the original cells of gram-positive bacterium), 0.2 to 500 parts by weight, 1 to 500 parts by weight, 5 to 500 parts by weight, 50 to 500 parts by weight, 0.2 to 100 parts by weight, 1 to 100 parts by weight, 5 to 100 parts by weight, 50 to 100 parts by weight, 0.2 to 100 parts by weight, 0.5 to 50 parts by weight or 1 to 20 parts by weight. The content of spices in the composition of the invention may be, in particular, with respect to 1 part by weight of the active ingredient contained in the composition of the present invention (calculated as the dry weight of the original cells of gram-positive bacterium), from 0.2 to 500 parts by weight, or from 50 to 500 parts by weight.

If the composition of the present invention contains a target substance (e.g. L-glutamic acid), the content of the target substance (e.g. L-glutamic acid) in the composition of the present invention may be, for example, 0.1% (w/w) or more, 0.2% (w/w) or more, 0.5% (w/w) or more 1% (w/w) or more, 2% (w/w) or more, 5% (w/w) or more, 10% (w/w) or more, or 20% (w/w) or more, and may be 90% (w/w) or less, 50% (w/w) or less, 20% (w/w) or less, 10% (w/w) or less, 5% (w/w) or less, 2% (w/w) or less, or 1% (w/w) or less, or any non-contradictory combination thereof. The content of the target substance (e.g. L-glutamic acid) in the composition of the present invention may specifically be, for example, from 0.2 to 90% (w/w), from 0.5 to 50% (w/w), from 1 to 50% (w/w), from 1 to 20% (w/w), from 5 to 50% (w/w), from 10 to 50% (w/w), or from 20 to 50% (w/w).

If the composition of the present invention contains a target substance (e.g. L-glutamic acid), the content of the target substance (e.g. L-glutamic acid) in the composition of the present invention may be, for example, with respect to 1 part by weight of the active ingredient of the composition of the present invention (calculated as the dry weight of the original cells of gram-positive bacterium), 0.1 parts by weight or more, 0.2 parts by weight or more, 0.5 parts by weight or more, 1 part by weight or more, 2 parts by weight or more, or 5 parts by weight or more, may be 50 parts by weight or less, 20 parts by weight or less, 10 parts by weight or less, 5 parts by weight or less, 2 parts by weight or less, or 1 part by weight or less, or may be a non-contradictory combination of them. The content of the target substance (e.g. L-glutamic acid) in the composition of the present invention may be, for example, specifically from 0.1 to 20 parts by weight with respect to 1 part by weight of the active ingredient contained in the composition of the present invention (calculated as the dry weight of the original cells of gram-positive bacterium).

When the composition of the present invention contains a culture of gram-positive bacterium as a component other than the active ingredient, the content of the culture of gram-positive bacterium in the composition of the present invention may be, for example, in terms of the amount of the original culture, 0.01% (w/w) or more, 0.1% (w/w) or more, 1% (w/w) or more, 5% (w/w) or more, or 10% (w/w) or more, may be 10000% (w/w) or less, 5000% (w/w) or less, 1000% (w/w) or less, 500% (w/w) or less, 300% (w/w) or less, 200% (w/w) or less, 150% (w/w) or less, 100% (w/w) or less, 70% (w/w) or less, 50% (w/w) or less, 30% (w/w) or less, 10% (w/w) or less, 5% (w/w) or less, or 1% (w/w) or less, or may be a non-contradictory combination thereof. The term “original culture” refers to a culture in which no concentration changes, such as concentration or dilution, have occurred after incubation; specifically, it may be the culture immediately after incubation. A content exceeding 100% (w/w) in terms of the amount of the original culture means that the culture is concentrated and contained in the object (in this case, the composition of the present invention). In other words, for example, a content of 200% (w/w) means that the culture is twice as concentrated and contained in the object.

The content of each component (i.e. the active ingredient and optionally other components) in the composition of the present invention can be set, for example, to obtain the amount of each component added in the method of the present invention described below.

Each of the ingredients (i.e. the active ingredient and optionally other components) contained in the compositions of the present invention may be mixed with each other and contained in the compositions of the present invention, or may be contained in the compositions of the present invention separately, or separately in any combination. For example, the compositions of the present invention may be provided as sets of individual components, each packaged separately. In such cases, the ingredients in the set may be used together as appropriate at the time of use.

### <3> Methods of the Invention.

The method of the present invention includes a step of utilising the active ingredient.

In other words, the method of the present invention is a method comprising a step of utilising the following component (A):
(A) lysozyme-treated product of a fraction containing cell walls of gram-positive bacterium.

By the method of the present invention, specifically by utilising the active ingredient, the flavor of the food product can be improved, i.e. a flavor-improving effect can be obtained. Thus, the method of the present invention may be implemented for improving the flavor of a food product. In other words, the method of the present invention may be, for example, a method for improving the flavor of a food product. The method is also referred to as the “flavor improvement method of the present invention”. The flavor improvement may be, for example, an enhancement of spiciness and/or kokumi. The flavor improvement may be, in particular, an enhancement of the pungency of the spice and/or the imparting of a thickness.

The method of the present invention may also be used to produce a food product with an improved flavor, specifically by utilising the active ingredient. Thus, the method of the present invention may be implemented for the production of a food product (specifically, the production of a food product with an improved flavor). That is, the method of the present invention may be, for example, a method of producing a food product (specifically, producing a food product with an improved flavor). The method is also referred to as the “method of producing the food product of the present invention”.

The active ingredient can be used for flavor improvement or in the production of food product by adding them to food ingredient during the production of the food product. In other words, the use of the active ingredient includes adding the active ingredient to the ingredient of the food product. In other words, the method of the present invention may specifically be, for example, a method of improving the flavor of a food product, including adding the active ingredient to an ingredient of the food product. The method of the present invention may also specifically be a method of producing a food product (specifically, producing a food product with an improved flavor), including, for example, adding the active ingredient to an ingredient of the food product. “Addition” is also referred to as “blending”.

The active ingredient may be utilised in the method of the present invention, for example in the form of the composition of the present invention. In other words, “utilisation of the active ingredient” includes the utilisation of the composition of the present invention. For example, “addition of the active ingredient” includes the addition of the composition of the present invention.

A food product obtained by the method of the present invention is also referred to as “food product of the present invention”. The food product of the present invention is specifically a food product with an improved flavor. The food product of the present invention is, in other words, a food to which an active ingredient has been added.

The flavor improvement or the production of the food product may be carried out in the same way as the production of ordinary food products, except, for example, for the utilisation of the active ingredient. In other words, the flavor improvement or the production of the food product may be carried out, for example, using the same ingredients and under the same production conditions as for ordinary food products, except that the active ingredient is utilised. Both the ingredients and the manufacturing conditions of the food product may be modified as appropriate and utilised in the flavor improvement or the production of the food product.

The type of food product is not restricted as long as the flavor improvement is desired. The food products encompass beverages. The food products also encompass seasonings. The food product may, for example, be liquid or solid. Examples of the food product specifically include beverages such as milk, soft drinks, alcoholic beverages and soups; processed meat products such as ham, sausages, dumplings, shumai, hamburger stakes, deep-fried meat and pork cutlets; processed seafood products such as salmon flakes, spicy cod roe, salted cod roe, grilled fish, dried fish, salted fish, fish sausages, fish paste, boiled fish, tsukudani, canned fish; confectionery such as crisps, potato snacks, corn snacks, wheat snacks, cinnamon cookies, rice crackers, arare, etc.; noodle soup such as udon soup, soba soup, somen soup, ramen soup, champon soup, pasta sauce; rice preparations such as rice balls, pilaf, fried rice, mixed rice, porridge, chazuke, etc; stewed dishes such as curry, stew, chili con carne, feijoata, mapo tofu; roux such as stew roux and curry roux; processed vegetable products such as kimchi and pickles; other processed foods such as bread, noodles, gratins, croquettes, mashed potatoes; sauces such as Chinese sauce, oyster sauce, cheese sauce, tomato sauce, white sauce, demi-glace sauce, curry sauce, genoa sauce, chilli sauce, tabasco sauce, etc.; seasoning oils such as raayu; basic seasonings such as soy sauce and miso; flavorings such as bonito, chicken, pork and beef flavorings; spicy seasonings such as shichimi chilli, soy bean sauce, gochujang, etc.; menu seasonings (seasonings specially tailored to the menu being cooked); and other seasonings such as dressings, miso, mayonnaise, tomato ketchup, consommé, etc. The term “soft drinks” may mean non-alcoholic beverages (beverages with an alcohol concentration of less than 1%), excluding milk and dairy products. Specific examples of soft drinks include water, fruit juices, vegetable juices, tea (e.g. chai, cinnamon tea), coffee drinks (e.g. coffee, milk drinks with coffee), carbonated drinks (e.g. ginger ale, lemon carbonated drinks) and sports drinks. Examples of the soups include, inter alia, dahl soup, tom yam kung, soup with egg, soup with wakame seaweed, soup with shark's fin, Chinese soup, consommé soup, curry-flavored soup, Japanese clear soup, miso soup and potage soup. Examples of the food product include, in particular, food products containing spices (e.g. the food products exemplified above, which contain spices). Examples of the food product also include spices themselves. The food product may be provided in a form that is ready to eat as they are, or in a form that requires preparation before or at the time of eating, such as concentrated or dried products. The food product is not limited to general foods, but also may be so-called health foods or medical foods, such as nutritional supplements, functional foods and foods for specified health uses. That is, for example, the food products illustrated above may be provided as general foods or as health foods or medical foods.

“Food ingredient” means a food material from which a food product is produced. The food ingredient is not restricted as long as the food product can be produced. The ingredient for the food product can be selected according to various conditions, for example, the type of food product. Examples of the ingredient for the food product include ingredients that can be normally used in the manufacture of food products, such as those exemplified above. Examples of the ingredient of the food product include, specifically, ingredients such as cereals, vegetables, meat, seafood and eggs; seasoning ingredients such as sugars, inorganic salts, organic acids, nucleic acids, amino acids and protein hydrolysates; dairy products such as milk and cheese; spices; flavorings; oils and fats; and alcohol. The organic acid, nucleic acid and amino acid may be those exemplified as target substances.

The active ingredient may be added to food ingredients at any stage of the food production process, as long as the flavor-improving effect is achieved. In other words, the “food ingredient” to which the active ingredient is added may be at any stage of the food production process. For example, the “food ingredient” to which the active ingredient is added may include a finished food product before the active ingredient is added. The active ingredient may be added to the food ingredient as it is or prepared in a desired form, such as a solution, as appropriate. The term “addition of the active ingredient” may collectively refer to the operation of bringing the active ingredient into coexistence with the food ingredient. The components other than the active ingredient may also be added to the food ingredient as appropriate. In other words, the method of the present invention may further comprise adding an ingredient other than the active ingredient to the ingredient of the food product. The component other than the active ingredient is not restricted as long as the flavor-improving effect is not lost (i.e. the flavor-improving effect by the active ingredient is obtained). The component other than the active ingredient may, for example, be selected according to various conditions, such as the type of food. Examples of the component other than the active ingredient include a culture of gram-positive bacterium, a target substance and a spice. The description of the addition of active ingredient can be applied mutatis mutandis to the addition of component other than the active ingredient. Each component (i.e. the active ingredient and optionally other component) may all be added to the food ingredient at the same time, or each may be added separately, or separately in any combination, to the food ingredient. The order in which each ingredient is added to the food ingredient is not restricted.

The amount and addition ratio of each component (i.e. the active ingredient and optionally other component) in the method of the present invention are not restricted as long as the flavor-improving effect is achieved. The amount and addition ratio of each component in the method of the present invention can be set according to various conditions, such as the type of food ingredients and the type of food product.

The active ingredient may be added to the food ingredients, for example, so that the desired range of the concentration of the active ingredient at the time of eating or drinking (e.g. the range of concentrations of the active ingredient at the time of eating or drinking as described below) is achieved.

The concentration of the active ingredient at the time of eating or drinking may be, for example, 0.005% (w/w) or more, 0.01% (w/w) or more, 0.02% (w/w) or more, 0.05% (w/w) or more, 0.1% (w/w) or more, 0.2% (w/w) or more, 0.5% (w/w) or more, 1% (w/w) or more, 2% (w/w) or more, 5% (w/w) or more, 10% (w/w) or more, 20% (w/w) or more, and may be 30% (w/w) or less, 20% (w/w) or less, 10% (w/w) or less, 5% (w/w) or less, 2% (w/w) or less, 1% (w/w) or less, 0.5% (w/w) or less, 0.2% (w/w) or less, 0.1% (w/w) or less, 0.05% (w/w) or less, 0.02% (w/w) or less, or 0.01% (w/w) or less, or a non-contradictory combination thereof. The content of the active ingredient in the composition of the present invention may specifically be, for example, from 0.005 to 0.01% (w/w), from 0.01 to 0.05% (w/w), from 0.05 to 0.1% (w/w), from 0.1 to 0.5% (w/w), from 0.5 to 1% (w/w), from 1 to 5% (w/w), or from 5 to 30% (w/w). The content of the active ingredient in the composition of the present invention may specifically be, for example, from 0.005 to 30% (w/w), from 0.05 to 20% (w/w), or from 0.5 to 20% (w/w).

The concentration of the active ingredient at the time of eating or drinking is, for example, in terms of dry weight of the original cells of gram-positive bacterium, 0.005% (w/w) or more, 0.007% (w/w) or more, 0.01% (w/w) or more, 0.02% (w/w) or more, 0.03% (w/w) or more, 0.04% (w/w) or more, 0.05% (w/w) or more, 0.07% (w/w) or more, 0.1% (w/w) or more, 0.2% (w/w) or more, 0.3% (w/w) or more, 0.4% (w/w) or more, 0.5% (w/w) or more, 0.7% (w/w) or more, or 1% (w/w) or more, may be 5% (w/w) or less, 2% (w/w) or less, 1% (w/w) or less, 0.7% (w/w) or less, 0.5% (w/w) or less, 0.4% (w/w) or less, 0.3% (w/w) or less, 0.2% (w/w) or less, 0.1% (w/w) or less, 0.07% (w/w) or less, 0.05% (w/w) or less, 0.04% (w/w) or less, 0.03% (w/w) or less, 0.02% (w/w) or less, or 0.01% (w/w) or less, or may be a non-contradictory combination thereof. The concentration of the active ingredient at the time of eating or drinking may specifically be, for example, from 0.005 to 0.01% (w/w), from 0.01 to 0.05% (w/w), from 0.05 to 0.1% (w/w), from 0.1 to 0.5% (w/w), from 0.5 to 1% (w/w), or from 1 to 5% (w/w) in terms of dry weight of the original cells of gram-positive bacterium. The concentration of the active ingredient at the time of eating or drinking may specifically be, for example, from 0.005 to 2% (w/w), from 0.005 to 1% (w/w), from 0.005 to 0.5% (w/w), from 0.01 to 2% (w/w), from 0.02 to 1% (w/w), or from 0.03 to 0.5% (w/w) in terms of dry weight of the original cells of gram-positive bacterium.

The description of the addition of active ingredient can be applied mutatis mutandis to the addition of the composition of the present invention. For example, the composition of the present invention can be added so that the amount of the active ingredient illustrated above is added.

The food product of the present invention may contain a spice in addition to the active ingredient. In other words, the food product of the present invention may be produced to contain the spice. In other words, the method of the present invention may further comprise adding the spice to the ingredient of the food product. The food product containing the spice may, for example, be produced by adding the spice itself or by adding a spice-containing material, such as a spice-containing seasoning. Where the composition of the present invention contains the spice, the spice may be added by addition of the composition of the present invention. The addition of the spice may be carried out in the same way as the addition of the active ingredient. The spice may be added to the ingredient of the food product, for example, so that the spice content in the food product of the present invention is in the desired range (e.g. the content range described below). The food ingredient may also intrinsically contain the spice.

If the food product of the present invention contains a spice, the content of the spice in the food product, for example, as the concentration at the time of eating or drinking, may be 0.01% (w/w) or more, 0.02% (w/w) or more, 0.03% (w/w) or more, 0.04% (w/w) or more, 0.05% (w/w) or more, 0.07% (w/w) or more, 0.1% (w/w) or more, 0.2% (w/w) or more, 0.3% (w/w) or more, 0.4% (w/w) or more, 0.5% (w/w) or more, 0.7% (w/w) or more, or 1% (w/w) or more, may also be 5% (w/w) or less, 2% (w/w) or less, 1% (w/w) or less, 0.7% (w/w) or less, 0.5% (w/w) or less, 0.4% (w/w) or less, 0.3% (w/w) or less, 0.2% (w/w) or less, 0.1% (w/w) or less, 0.07% (w/w) or less, or 0.05% (w/w) or less, or may be a non-contradictory combination thereof. Specifically, the content of the spice in the food product of the present invention may, for example, as the concentration at the time of eating or drinking, be from 0.01 to 0.05% (w/w), from 0.05 to 0.1% (w/w), from 0.1 to 0.5% (w/w), from 0.5 to 1% (w/w), or from 1 to 5% (w/w). Specifically, the content of the spice in the food product of the present invention may, for example, as the concentration at the time of eating or drinking, be from 0.01 to 2% (w/w), from 0.02 to 1% (w/w), or from 0.03 to 0.5% (w/w).

If the food product of the present invention contains the spice, the content of the spice in the food product of the present invention is, for example, with respect to 1 part by weight of the active ingredient (in terms of dry weight of the cells of gram-positive bacterium) contained in the food product of the present invention, 0.2 parts by weight or more, 0.5 parts by weight or more, 1 part by weight or more, 2 parts by weight or more, 5 parts by weight or more, 10 parts by weight or more, 20 parts by weight or more, 50 parts by weight or more, or 100 parts by weight or more, and may be 1000 parts by weight or less, 500 parts by weight or less, 200 parts by weight or less, 100 parts by weight or less, 50 parts by weight or less, 20 parts by weight or less, 10 parts by weight or less, 5 parts by weight or less, or 2 parts by weight or less, or may be a non-contradictory combination of them. The spice content in the food product of the present invention may be, for example, from 0.2 to 500 parts by weight, from 1 to 500 parts by weight, from 5 to 500 parts by weight, from 50 to 500 parts by weight, from 0.2 to 100 parts by weight, from 1 to 100 parts by weight, from 5 to 100 parts by weight, from 50 to 100 parts by weight, from 0.2 to 100 parts by weight, from 0.5 to 50 parts by weight, or from 1 to 20 parts by weight. The spice content in the food product of the present invention may be, in particular, from 0.2 to 500 parts by weight, or from 50 to 500 parts by weight with respect to 1 part by weight of the active ingredient (in terms of dry weight of the cells of gram-positive bacterium) contained in the food product of the present invention.

In addition to the active ingredient, the food product of the present invention may contain a target substance. In other words, the food product of the present invention may be produced to contain the target substance. In other words, the method of the present invention may further comprise adding the target substance to the ingredient of the food product. The target substance is described above. A food product containing the target substance may, for example, be produced by adding the target substance itself or by adding a material containing the target substance, such as a seasoning containing the target substance. The term “adding the active ingredient and the target substance” is not limited to cases where the active ingredient and the target substance obtained individually are added, but also includes cases where the active ingredient and the target substance obtained collectively are added. A case in which the active ingredient and the target substance obtained collectively are added is when a culture containing both the target substance and the gram-positive bacterium is subjected to lysozyme treatment to prepare the active ingredient and add it. If the composition of the present invention contains the target substance, the target substance may be added by addition of the composition of the present invention. The addition of the target substance can be carried out in the same way as the addition of the active ingredient. The target substance may be added to the ingredient of the food product, for example, so that the content of the target substance in the food product of the present invention is in the desired range (e.g. the content range described below). The ingredient of the food product may also intrinsically contain the target substance.

If the food product of the present invention contains a target substance (e.g., L-glutamic acid), the content of the target substance (e.g., L-glutamic acid) in the food product of the present invention may, for example, as the concentration at the time of eating or drinking, be 0.01% (w/w) or more, 0.02% (w/w) or more, 0.03% (w/w) or more, 0.04% (w/w) or more, 0.05% (w/w) or more, 0.07% (w/w) or more, 0.1% (w/w) or more, 0.2% (w/w) or more, 0.3% (w/w) or more, 0.4% (w/w) or more, or 0.5% (w/w) or more, may be 5% (w/w) or less, 2% (w/w) or less, 1% (w/w) or less, 0.7% (w/w) or less, 0.5% (w/w) or less, 0.4% (w/w) or less, 0.3% (w/w) or less, 0.2% (w/w) or less, or 0.1% (w/w) or less, and may be a non-contradictory combination thereof. Specifically, the content of the target substance (e.g., L-glutamic acid) in the food product of the present invention may, for example, as the concentration at the time of eating or drinking, be from 0.01 to 2% (w/w), from 0.02 to 1% (w/w), or from 0.03 to 0.5% (w/w).

If the food product of the present invention contains a target substance (e.g. L-glutamic acid), the content of the target substance (e.g. L-glutamic acid) in the food product of the present invention may be, for example, with respect to 1 part by weight of the active ingredient contained in the food product of the present invention (converted to dry weight of the cells of gram-positive bacterium), 0.1 parts by weight or more, 0.2 parts by weight or more, 0.5 parts by weight or more, 1 part by weight or more, 2 parts by weight or more, or 5 parts by weight or more, may be 50 parts by weight or less, 20 parts by weight or less, 10 parts by weight or less, 5 parts by weight or less, 2 parts by weight or less, or 1 part by weight or less, or may be a non-contradictory combination thereof. The content of the target substance (e.g. L-glutamic acid) in the food product of the present invention may be, for example, specifically from 0.1 to 20 parts by weight with respect to 1 part by weight of the active ingredient in the food product of the present invention (in terms of dry weight of the cells of gram-positive bacterium).

In addition to the active ingredient, the food product of the present invention may contain a culture of gram-positive bacterium. In other words, the food product of the invention may be produced to contain a culture of gram-positive bacterium. In other words, the method of the present invention may further comprise adding a culture of gram-positive bacterium to an ingredient of the food product. Cultures of gram-positive bacterium as ingredients other than the active ingredient are described above. The food product containing the culture of gram-positive bacterium may, for example, be produced by adding the culture of gram-positive bacterium themselves or by adding a material containing the culture of gram-positive bacterium. The term “adding an active ingredient and a culture of gram-positive bacterium” is not limited to cases where the active ingredient and the culture obtained individually are added, but also includes cases where the active ingredient and the culture obtained collectively are added. The case of adding the active ingredient and the culture obtained collectively includes the case where the culture containing the cells of gram-positive bacterium is subjected to lysozyme treatment to prepare the active ingredient and add it. If the composition of the present invention contains a culture of gram-positive bacterium, the culture of gram-positive bacterium may be added by addition of the composition of the present invention. The addition of the culture of gram-positive bacterium can be carried out in the same way as the addition of the active ingredient. The culture of gram-positive bacterium may be added to the ingredient of the food product, for example, so that the content of the culture of gram-positive bacterium in the food product of the present invention is in the desired range (e.g. the content range described below). The food ingredient may also intrinsically contain a culture of gram-positive bacterium.

If the food product of the present invention contains a culture of gram-positive bacterium as a component other than the active ingredient, the content of the culture of gram-positive bacterium in the food product of the present invention may be, for example, in terms of the amount of the original culture, 0.01% (w/w) or more, 0.1% (w/w) or more, 1% (w/w) or more, 5% (w/w) or more, or 10% (w/w) or more, may be 10000% (w/w) or less, 5000% (w/w) or less, 1000% (w/w) or less, 500% (w/w) or less, 300% (w/w) or less, 200% (w/w) or less, 150% (w/w) or less, 100% (w/w) or less, 70% (w/w) or less, 50% (w/w) or less, 30% (w/w) or less, 10% (w/w) or less, 5% (w/w) or less, or 1% (w/w) or less, or may be a non-contradictory combination thereof.

### <4> Use of Active Ingredients

The present specification also discloses the use of the active ingredient in the above exemplified uses. That is, the present specification discloses, for example, the use of active ingredient for flavor improvement or for the production of food product and the use of active ingredient in the production of composition for flavor improvement or for the production of food products.

The present specification also discloses an active ingredient for use in the above-mentioned examples. That is, the specification discloses, for example, an active ingredient for use in improving flavor or in the production of food product, or active ingredient for use in the production of a composition for flavor improvement or for the production of food products.

The present specification also discloses the use of active ingredient for use in combination with other components (e.g. spices). The present specification also discloses an active ingredient for use in combination with other components (e.g. spices). The active ingredient may be used in combination with other components (e.g. spices) for the uses illustrated above.

### Example

The invention is described in more detail below with reference to non-limiting examples. In this example, “L-Glu” or “Glu” means L-glutamic acid.

### Example 1: Acquisition of a High L-Glutamic Acid Producing Strain from Corynebacterium casei JCM 12072

(1-1) Creation of Mutant Strain Library of C. casei JCM 12072

C. casei JCM 12072 (wild-type strain) was inoculated into shaking flasks filled with 30 mL of the media listed in Table 2(A), incubated at 30° C. for 1 day with shaking, and the bacterial cells were collected. The bacterial cells were suspended in a solution containing 0.1 M potassium phosphate buffer (pH 7.0), 6.0% dimethyl sulphoxide and 0.1 mg/mL N-methyl-N-nitrosoguanidine (NTG) and allowed to stand for 50 min at room temperature. The bacterial cells were collected and washed three times with 0.1 M potassium phosphate buffer (pH 7.0). The bacterial cells were incubated for 2 h at 30° C. in shaking flasks filled with 30 mL of the recovery culture medium listed in Table 2 (B) and the bacterial cells were collected. The bacterial cells were suspended in 20% glycerol and stored at −80° C. This was used as the mutant strain library.

TABLE 2
(A)

| Component | Final concentration | |
|---|---|---|
| Soy peptone | 40 | g/L |
| Yeast extract | 8 | g/L |
| MgSO₄•7H₂O | 1 | g/L |

pH 7.5 (KOH), 120° C., autoclaved for 20 minutes

(B)

| Component | Final concentration | |
|---|---|---|
| Soy peptone | 40 | g/L |
| Yeast extract | 8 | g/L |
| MgSO₄•7H₂O | 1 | g/L |
| Na₂C₄H₄O₄•6H₂O | 2 | g/L |

pH 7.0 (KOH), 120° C., autoclaved for 20 minutes

(1-2) Screening of high L-glutamic acid producing strains from mutant strain library

The mutant strain library prepared in (1-1) was then incubated in the Glu minimal medium listed in Table 3 supplemented with 100 mg/L ampicillin for 29 h at 30° C. with shaking, and then seeded on the agar medium listed in Table 2(A). The growing colonies were inoculated on the Glu minimal medium listed in Table 3 and the agar medium listed in Table 2(A), respectively, and 26 clones with reduced L-glutamic acid (L-Glu) capitalisation were selected as candidate strains.

TABLE 3

| | |
|---|---|
| Component | Final concentration |

| | | |
|---|---|---|
| KH₂PO₄ | 3 | g/L |
| NaCl | 0.5 | g/L |
| Na₂HPO₄ | 6.78 | g/L |
| NH₄Cl | 1 | g/L |
| Sodium hydrogen L(+)-glutamic acid•H₂O | 4.49 | g/L |
| MgSO₄ | 0.24 | g/L |
| CaCl₂ | 0.01 | g/L |
| Biotin | 0.1 | μg/L |

pH not adjusted, sterilized by filtration

Twenty-six candidate strains were incubated in 500 μL of evaluation medium (Table 4) filled in 96 deep-well plates with shaking at 30° C. for 48 hours and the amount of L-Glu accumulated in the medium was measured using a Biotech Analyser AS210 (Sakura SI Co., Ltd.). The RUN5-2-96 strain (NITE BP-03688) with the highest L-Glu concentration among the evaluated strains was selected. RUN5-2-96 strain is also referred to as “AJ111891 strain”. RUN5-2-96 strain (AJ111891) has all 135 mutations of group A (i.e. all of mutations A-1 to A-135).

TABLE 4
<part A>

| Component | Final concentration |
|---|---|
| Glucose | 55 g/L |

pH not adjusted, autoclaved at 120° C. for 20 minutes

<part B>

| Component | Final concentration | |
|---|---|---|
| (NH₄)₂SO₄ | 5 | g/L |
| KH₂PO₄ | 1 | g/L |
| MgSO₄•7H₂O | 0.4 | g/L |
| FeSO₄•7H₂O | 0.01 | g/L |
| MnSO₄•5H₂O | 0.01 | g/L |
| VB1 | 0.0002 | g/L |
| Biotin | 0.0006 | g/L |
| Bacto Peptone | 10 | g/L |
| Yeast Extract | 5 | g/L |

pH 7.5 (KOH), autoclaved at 120° C. for 20 minutes
Mixed in a ratio of 20% for part A and 80% for part B.

(1-3) Acquisition of a High L-Glutamic Acid Producing Strain from Strain RUN5-2-96.

Using the RUN5-2-96 strain (AJ111891) as the parent strain, further mutations were introduced by mutation treatment to obtain a mutant strain with improved L-Glu producing ability. The obtained mutant strain is hereafter also referred to simply as “AJ111891-derived mutant strain”. The AJ111891-derived mutant strain has all 135 mutations in Group A and all 92 mutations in Group B.

### Example 2: Preparation of Dried Cells of C. casei JCM 12072 and AJ111891-Derived Mutant Strain

Cultures of C. casei JCM 12072 and AJ111891-derived mutant strain were performed by jar culture using the media listed in Table 5, respectively. A 1 L-volume fermenter was used for jar culture. The culture temperature was maintained at a constant 30° C., the culture pH was controlled to 6.8 with ammonia gas and the dissolved oxygen concentration was controlled by agitation control to be 23% or more as a relative value with the saturated dissolved oxygen concentration as 100%. The culture medium was sterilised at 80° C. for 20 min, and the culture medium components were removed by centrifugation to obtain a cell pellet. Furthermore, the bacterial cells were washed by adding the same volume of water as the culture medium and the supernatant was removed by centrifugation, which was repeated twice. After washing, the cell pellet was frozen at −80° C. and the water was removed by a freeze-dryer to obtain dried bacterial cells of C. casei JCM 12072 and the AJ111891-derived mutant strain.

TABLE 5

| Component | Final concentration | |
|---|---|---|
| Potato peptone | 10 | g/L |
| Yeast Extract | 10 | g/L |
| Glucose | 80 | g/L |
| Biotin | 3 | mg/L |
| KH₂PO₄ | 1 | g/L |
| Ammonium sulfide | 1 | g/L |
| Deformer (O-50D) | 0.4 | mL/L |

### Example 3: Preparation of Fermented Seasonings Using C. casei AJ111891 Strain and AJ111891-Derived Mutant Strain

(3-1)

The following procedure was used for L-Glu fermentation by C. casei AJ111891 and AJ111891-derived mutant strain.

First, seed cultures of AJ111891 and AJ111891-derived mutant strain were carried out by jar culture using the seed media listed in Table 6, respectively. The culture temperature was maintained at a constant 30° C. and the culture pH was maintained at 6.8 with ammonia gas. The main culture of the AJ111891 strain and the AJ111891-derived mutant strain was then conducted by jar culture using the media listed in Table 7. As in the seed culture, the culture pH was controlled at 6.8 using ammonia gas. Until the OD620 reached 40, the culture temperature was maintained at 30° C. and the dissolved oxygen concentration was 23% or more. When OD620 reached 40, the temperature was increased to 35° C. and incubation continued until the glucose was exhausted. In this way, cultures of the AJ111891 strain and the AJ111891-derived mutant strain containing L-Glu were obtained. The amount of L-Glu accumulated in the medium and the amount of glucose in the medium were measured using a Biotech Analyser AS210 (Sakura SI Co., Ltd.). Dry cell weight (DCW) was measured by preparing dried bacterial cells from the culture medium using the same procedure as in Example 2 (but without sterilisation).

TABLE 6

| Component | Final concentration | |
|---|---|---|
| Glucose | 45 | g/L |
| KH₂PO₄ | 3.52 | g/L |
| MgSO₄•7H₂O | 0.45 | g/L |
| FeSO₄•7H₂O | 10 | mg/L |
| Mameno | 1540 | mg/L |
| Disodium succinate | 2 | g/L |
| L(+)-ascorbic acid | 8.55 | mg/L |
| VB1•HCl | 23 | mg/L |
| VB12 | 4 | μg/L |
| Biotin | 3.2 | mg/L |
| Yeast Extract | 5 | g/L |
| Deformer (PP-AJ-2K) | 0.1 | mL/L |

TABLE 7

| Component | Final concentration | |
|---|---|---|
| Glucose | 100 | g/L |
| KH₂PO₄ | 3.46 | g/L |
| MgSO₄•7H₂O | 0.5 | g/L |
| FeSO₄•7H₂O | 10 | mg/L |
| Mameno | 500 | mg/L |
| Disodium succinate | 2 | g/L |
| L(+)-ascorbic acid | 8.55 | mg/L |
| VB1•HCl | 23 | mg/L |
| VB12 | 4 | μg/L |
| Biotin | 0.5 | mg/L |
| Deformer (PP-AJ-2K) | 0.2 | mL/L |
| Betaine | 0.6 | g/L |

(3-2)

Each culture solution obtained in (3-1) was transferred to a 2 L Durand bottle and sterilised by stirring for 10 min after reaching 70° C. in a water bath. The sterilised cultures were then aliquoted into centrifuge tubes (Himac 500PA 330437A) and centrifuged using a centrifuge (Hitachi CR20 GIII, PRP9-2 rotor) at 7,000 rpm (9,400×G), 25° C. for 10 min, followed by decanting and collecting the supernatant with electronic pipettes. To the bacterium remaining in the centrifuge tube from the bacterium separation operation, pure water was added in an amount equal to half the weight of the culture medium and suspended using a vortex, followed by centrifugation using a centrifuge (Hitachi CR20 GIII, PRP9-2 rotor) at 7,000 rpm (9,400×G), 25° C., 10 minutes. After centrifugation, the supernatant was collected by decantation and electronic pipette. By carrying out this operation twice, the components other than the bacterial cells were separated as supernatant and the washed bacterium of strain AJ111891 and the AJ111891-derived mutant strain were obtained.

(3-3)

The supernatant collected in (3-2) was transferred to a 2 L Durand bottle, heated in a water bath, and after reaching 60° C., granular activated carbon (NORIT (R) GAC1240 by Cabot NORIT) was added in an amount equal to the L-Glu in the liquid and stirred for 1 hour. The activated carbon was then filtered through a circular quantitative filter paper No. 5C (ADVANTEC Toyo), a Buchner funnel and a filter bottle to obtain filtrate. Activated carbon fines leaking into the filtrate were removed by dispensing the filtrate into centrifuge tubes (Himac 500PA 330437A) and by subjecting it to centrifugation using a centrifuge (Hitachi CR20 GIII, PRP9-2 rotor) at 7,000 rpm (9,400×G), 25° C. for 10 min, and by collecting the supernatant by decantation.

(3-4)

To the filtrate obtained in (3-3), 27% sodium hydroxide solution was added (1.3 times the substance amount of sodium hydroxide to L-Glu in the liquid) and decompression concentration was carried out using a rotary evaporator at a decompression degree of 90 hPa and a water bath temperature of 70° C. Each time the liquid volume was reduced to about ⅓, pure water of ⅔ of the liquid volume before concentration was added and concentrated under reduced pressure. This operation was repeated twice. Thereafter, the decompression concentration was carried out until the liquid volume was reduced to about ⅓. The remaining fine powder in the concentrated liquid was removed using circular quantitative filter paper No. 5C (ADVANTEC Toyo), a Buchner funnel and a filter bottle to obtain concentrates of the culture supernatant of the AJ111891 strain and the AJ111891-derived mutant strain.

(3-5)

To the concentrated culture supernatant of C. casei AJ111891 and AJ111891-derived mutant strains obtained in (3-4), respectively, were added the washed bacterium of C. casei AJ111891 and AJ111891-derived mutant strains obtained in (3-2). The addition ratio was matched to the separation ratio when the culture medium before the separation of the bacterium was separated into sterilised liquid and bacterial cells. Maltodextrin (Ciranda, Conventional Non-GMO Tapioca Maltodextrin DE10) was added to them to achieve a solids content of 97% and L-Glu content of 35% in the product powder after spray drying. These were spray-dried using a spray dryer (Nihon BUCHI K.K., B-290) at an inlet temperature of 180° C., a flow rate of 5 mL/min, a compressed gas flow rate of 473 L/h and a hot air volume of 38 m³/h to produce a fermented seasoning containing the bacterial cells of C. casei AJ111891 strain (hereinafter also referred to as “C. casei AJ111891 strain fermentation broth/bacterial cells-containing seasoning”) and fermented seasoning containing the bacterial cells of the mutant strain derived from C. casei AJ111891 (hereinafter referred to as “C. casei AJ111891-derived mutant strain fermentation broth/bacterial cells-containing seasoning”) were obtained. C. casei AJ111891 strain fermented broth/bacterial cells-containing seasoning contains 6% by weight of the bacterial cells of the C. casei AJ111891 strain. The C. casei AJ111891-derived mutant strain fermented broth/bacterial cells-containing seasoning contains 6% by weight of bacterial cells of the C. casei AJ111891-derived mutant strain.

### Example 4: Effect of Enzyme Treatment of Corynebacterium casei Bacterium on the Pungency Enhancing Effect

A suspension was obtained by mixing 0.2 g of dried bacterial cells of the Corynebacterium casei AJ111891-derived mutant strain obtained in Example 2 with 19.0 g of distilled water. Lysozyme BIO (BIOCON (JAPAN) LTD., derived from egg white; 39 000 FPI units/mg) or papain (Amano Enzyme) was added to this suspension to reach a weight of 1/3000 to 1/10th of the cell weight or 1/1000 to 1/10th of the cell weight. Distilled water was added as appropriate to bring the total weight of the enzyme reaction solution to 20 g. After the addition of the enzyme solution, the reaction was carried out at 45° C. for 4 h. After the reaction, heating was carried out at 100° C. for 10 min and the enzyme reaction solution of the bacterium was obtained by ice cooling. Samples for sensory evaluation were obtained by mixing 10 g of the reaction solution with 90 g of curry sauce (17.2 g of curry roux manufactured by House Foods Corp. mixed with 400 g of hot water). The sample mixed with 10 g of distilled water and 90 g of curry sauce was used as the negative control (NC). The positive control (PC) was made by mixing 10 g of a 1% suspension of enzyme-untreated bacterial cells with 90 g of curry sauce. The sensory evaluation was carried out by a joint rating method by two expert panel members. The pungency intensity of the negative control was scored 0 and the pungency intensity when the enzyme-untreated bacterial cells were added was scored 100. It was separately confirmed that the pungency of mashed potatoes containing 0.2% by weight of chilli pepper was comparable to that of mashed potatoes containing 0.4% by weight of chilli pepper when 0.1% of the enzyme-untreated bacterial cells were added to the mashed potatoes containing 0.2% by weight of chilli pepper.

The results are shown in Table 8. The reaction solution obtained by adding lysozyme at 1/10000th to 1/10th of the weight of the bacterial cells showed a stronger pungency-enhancing effect than the enzyme-untreated bacterial cells. On the other hand, the reaction solution obtained by adding lysozyme at 1/30000th of the cell weight showed a pungency-enhancing effect comparable to that of the enzyme-untreated bacterial cells. The reaction solution obtained by adding papain showed a pungency-enhancing effect comparable to that of the enzyme-untreated bacterial cells. These results indicate that, under the present conditions, the pungency-enhancing effect was improved by adding lysozyme to the Corynebacterium casei bacterium at a concentration of 1/10000th to 1/10th of the cell weight in the enzymatic reaction.

TABLE 8

| | Enzyme amount | | |
|---|---|---|---|
| | (with respect | | |
| | to bacterial | | |
| Sample | cell weight) | Pungency | Comment |
| NC(without bacterial cells) | — | 0 | |
| PC (0.1% untreated bacterial cells) | — | 100 | |
| Lysozyme-treated bacterial cells | 1/30000 | 100 | equivalent to PC |
| Lysozyme-treated bacterial cells | 1/10000 | 110 | slightly stronger pungency than PC |
| Lysozyme-treated bacterial cells | 1/3000 | 140 | a little stronger pungency than PC |
| Lysozyme-treated bacterial cells | 1/1000 | 160 | stronger pungency than PC |
| Lysozyme-treated bacterial cells | 1/100 | 200 | stronger pungency than PC |
| Lysozyme-treated bacterial cells | 1/50 | 200 | stronger pungency than PC |
| Lysozyme-treated bacterial cells | 1/10 | 160 | stronger pungency than PC, while |
| | | | having sweetening properties |
| Papain-treated bacterial cells | 1/1000 | 100 | equivalent to PC |
| Papain-treated bacterial cells | 1/100 | 100 | equivalent to PC |
| Papain-treated bacterial cells | 1/50 | 100 | equivalent to PC |
| Papain-treated bacterial cells | 1/10 | 100 | equivalent to PC |

### Example 5: Effect of Enzyme Treatment of Corynebacterium casei Bacterium on their Thickness-Enhancing Effect

The enzyme reaction solutions of the bacterial cells obtained in Example 4 were used to investigate the thickness-enhancing effect in a cheese sauce system. A sample for sensory evaluation was obtained by mixing 10 g of the obtained reaction solution with 90 g of cheese sauce (Mizkan, RAGU Four Cheese). A negative control (NC) was also obtained by mixing 10 g of distilled water with 90 g of cheese sauce. The positive control (PC) was made by mixing 10 g of a 1% suspension of enzyme-untreated bacterial cells with 90 g of cheese sauce. The sensory evaluation was carried out by a joint rating method by two expert panel members. The thickness intensity of the negative control was given a score of 0, and the thickness intensity when the enzyme-untreated bacterial cells were added was given a score of 100.

The results are shown in Table 9. The reaction solutions obtained by adding lysozyme at 1/10000th to 1/10th of the weight of the bacterium showed a stronger thickness-enhancing effect than the enzyme-untreated bacterium. On the other hand, the reaction solution obtained by adding lysozyme at 1/30000th of the weight of the bacterial cells showed a similar thickness-enhancing effect to that of the enzyme-untreated bacterium. The reaction solution obtained by adding papain also showed a similar thickness-enhancing effect to that of the enzyme-untreated bacterium. These results indicate that under the present conditions, the thickness-enhancing effect is improved by adding lysozyme to the Corynebacterium casei bacterium at a concentration of 1/10000th to 1/10th of the cell weight in the enzymatic reaction.

TABLE 9

| | Enzyme amount | | |
|---|---|---|---|
| | (with respect | | |
| | to bacterial | | |
| Sample | cell weight) | Thickness | Comment |
| NC(without bacterial cells) | — | 0 | |
| PC (0.1% untreated bacterial cells) | — | 100 | |
| Lysozyme-treated bacterial cells | 1/1000 | 130 | stronger thickness than PC |
| Lysozyme-treated bacterial cells | 1/100 | 150 | stronger thickness than PC |
| Lysozyme-treated bacterial cells | 1/50 | 130 | stronger thickness than PC |
| Lysozyme-treated bacterial cells | 1/10 | 110 | rendering sweetness and |
| | | | off-flavor |
| Papain-treated bacterial cells | 1/1000 | 100 | almost equivalent to PC |
| Papain-treated bacterial cells | 1/100 | 100 | almost equivalent to PC |
| Papain-treated bacterial cells | 1/50 | 100 | almost equivalent to PC |
| Papain-treated bacterial cells | 1/10 | 100 | almost equivalent to PC |

### Example 6: Effect of Enzyme Treatment of Corynebacterium casei (Wild Strain) on Pungency Enhancing Effect

A suspension was obtained by mixing 0.2 g of dried bacterial cells of Corynebacterium casei JCM 12072 strain (wild strain) obtained in Example 2 with 19.0 g of distilled water. To this suspension, lysozyme (BIOCON (JAPAN) LTD., derived from egg white) was added at 1/100th and 1/100th of the cell weight. Distilled water was added as appropriate to bring the total weight of the enzyme reaction solution to 20 g. After the addition of the enzyme solution, the reaction was carried out at 50° C. for 6 h. After the reaction, heating was carried out at 100° C. for 10 min and the solution was ice-cooled to obtain the enzyme reaction solution of the bacterial cells. A sample for sensory evaluation was obtained by mixing 10 g of the reaction solution with 90 g of curry sauce (17.2 g of curry roux manufactured by House Foods Corp. mixed with 400 g of hot water). The sample mixed with 10 g of distilled water and 90 g of curry sauce was used as the negative control (NC). The positive control (PC) was made by mixing 10 g of a 1% suspension of enzyme-untreated bacterial cells with 90 g of curry sauce. The sensory evaluation was carried out by a joint rating method by two expert panel members. The pungency intensity of the negative control was scored 0 and the pungency intensity when the enzyme-untreated bacterial cells were added was scored 100. It was separately confirmed that the pungency of mashed potatoes containing 0.2% by weight of chilli pepper was comparable to that of mashed potatoes containing 0.4% by weight of chilli pepper when 0.1% of the enzyme-untreated bacterial cells were added to the mashed potatoes containing 0.2% by weight of chilli pepper.

The results are shown in Table 10. The reaction solutions obtained by adding lysozyme at 1/1000th and 1/100th of the cell weight showed a stronger pungency-enhancing effect compared to the enzyme-untreated bacterium. These results indicate that, under the present conditions, the pungency-enhancing effect of the enzymatic reaction of Corynebacterium casei (wild strain) with lysozyme at 1/1000th or 1/100th of the weight of the bacterium is enhanced by the addition of lysozyme.

TABLE 10

| | Enzyme amount | | |
|---|---|---|---|
| | (with respect | | |
| | to bacterial | Pun- | |
| Sample | cell weight) | gency | Comment |
| NC(without bacterial | — | 0 | |
| cells) | | | |
| PC (0.1% untreated | — | 100 | |
| bacterial cells) | | | |
| Lysozyme-treated | 1/1000 | 180 | stronger pungency |
| bacterial cells | | | than PC |
| Lysozyme-treated | 1/100 | 220 | stronger pungency |
| bacterial cells | | | than PC |

### Example 7: Effect of Enzyme Treatment of Corynebacterium casei (Wild Strain) on the Thickness-Enhancing Effect

The enzyme reaction solutions of the bacterial cells obtained in Example 6 were used to investigate the thickness-enhancing effect in a cheese sauce system. A sample for sensory evaluation was obtained by mixing 10 g of the reaction solution with 90 g of cheese sauce (Mizkan, RAGU Four Cheese). A negative control (NC) was also obtained by mixing 10 g of distilled water with 90 g of cheese sauce. The positive control (PC) was made by mixing 10 g of a 1% suspension of enzyme-untreated bacterial cells with 90 g of cheese sauce. The sensory evaluation was carried out by a joint rating method by two expert panel members. The thickness intensity of the negative control was given a score of 0, while the thickness intensity when the enzyme-untreated bacterial cells were added was given a score of 100.

The results are shown in Table 11. The reaction solutions obtained by adding lysozyme at 1/1000th and 1/100th of the weight of the bacterial cells showed a strong thichness-enhancing effect compared to the enzyme-untreated bacterium. These results indicate that under the present conditions, the addition of lysozyme to Corynebacterium casei (wild strain) at 1/1000 or 1/100th of the cell weight in the enzymatic reaction improves the thickness-enhancing effect.

TABLE 11

| | Enzyme amount | | |
|---|---|---|---|
| | (with respect | | |
| | to bacterial | Thick- | |
| Sample | cell weight) | ness | Comment |
| NC(without bacterial | — | 0 | |
| cells) | | | |
| PC (0.1% untreated | — | 100 | |
| bacterial cells) | | | |
| Lysozyme-treated | 1/1000 | 160 | stronger thickness |
| bacterial cells | | | than PC |
| Lysozyme-treated | 1/100 | 180 | stronger thickness |
| bacterial cells | | | than PC |

### Example 8: Effect of Enzyme Treatment of Corynebacterium casei Fermentation Broth/Bacterial Cells-Containing Seasoning and Fungi on the Pungency Enhancing Effect

A suspension was obtained by mixing 0.4 g of the C. casei AJ111891 strain fermentation broth/bacterial cells-containing seasoning (6.0% cell content) obtained in Example 3 with 19.0 g of distilled water. Lysozyme (BIOCON (JAPAN) LTD., derived from egg white) was added to the suspension to a weight of 1/1000th to 1/10th of the cell weight. Distilled water was added as necessary to bring the total weight of the enzyme reaction solution to 20 g. After the addition of the enzyme solution, the reaction was carried out at 50° C. for 6 h. After the reaction, heating was carried out at 100° C. for 10 min and the solution was ice-cooled to obtan enzyme reaction solution of seasoning. A sample for sensory evaluation was obtained by mixing 10 g of the reaction solution with 90 g of curry sauce (17.2 g of curry roux manufactured by House Foods Corp. mixed with 400 g of hot water). The sample mixed with 10 g of distilled water and 90 g of curry sauce was used as the negative control (NC). A positive control (PC) was obtained by mixing 10 g of a 2% suspension of enzyme-untreated C. casei AJ111891 strain fermentation broth/bacterial cells-containing seasoning with 90 g of curry sauce. The sensory evaluation was carried out by a joint rating method by two expert panel members. The pungency intensity of the negative control was given a score of 0, while the pungency intensity when the enzyme-untreated seasoning was added was given a score of 100.

The results are shown in Table 12. The reaction solutions obtained by adding lysozyme to the Corynebacterium casei fermentation broth at 1/10000th to 1/10th of the cell weight showed a stronger pungency-enhancing effect than the enzyme-untreated seasonings. These results indicate that, under the present conditions, the pungency-enhancing effect is improved by adding lysozyme to the Corynebacterium casei fermentation broth/bacterial cells-containing seasoning at 1/10000th to 1/10th of the cell weight in the enzymatic reaction.

TABLE 12

| | Enzyme amount | | |
|---|---|---|---|
| | (with respect | | |
| | to bacterial | Pun- | |
| Sample | cell weight) | gency | Comment |
| NC(without | — | 0 | |
| bacterial cells) | | | |
| PC (0.2% | — | 100 | |
| untreated | | | |
| seasoning) | | | |
| Lysozyme-treated | 1/10000 | 130 | a little stronger pungency |
| seasoning | | | than PC |
| Lysozyme-treated | 1/1000 | 170 | stronger pungency than PC |
| seasoning | | | |
| Lysozyme-treated | 1/100 | 200 | stronger pungency than PC |
| seasoning | | | (about twice as strong) |
| Lysozyme-treated | 1/50 | 200 | stronger pungency than PC |
| seasoning | | | (about twice as strong) |
| Lysozyme-treated | 1/10 | 200 | stronger pungency than PC |
| seasoning | | | (about twice as strong) |

### Example 9: Effect of Enzyme Treatment of Corynebacterium casei Fermentation Broth/Bacterial Cells-Containing Seasoning on their Thickness-Enhancing Effect

The enzyme reaction solution of the seasoning obtained in Example 8 was used to investigate the thickness-enhancing effect in a cheese sauce system. A sample for sensory evaluation was obtained by mixing 10 g of the reaction solution with 90 g of cheese sauce (Mizkan, RAGU Four Cheese). A negative control (NC) was also obtained by mixing 10 g of distilled water with 90 g of cheese sauce. The positive control (PC) was made by mixing 10 g of a 2% suspension of enzyme-untreated C. casei AJ111891 strain fermentation broth/bacterial cells-containing seasoning with 90 g of cheese sauce. The sensory evaluation was carried out by a joint rating method by two expert panel members. The thickness intensity of the negative control was given a score of 0, while the thickness intensity when the enzyme-untreated seasoning was added was given a score of 100.

The results are shown in Table 13. The reaction solutions obtained by adding lysozyme to the Corynebacterium casei fermentation broth at 1/10000th to 1/10th of the weight of the bacterial cells showed a stronger thickness-enhancing effect than the enzyme-untreated seasonings. These results indicate that under the present conditions, the addition of lysozyme to the Corynebacterium casei fermentation broth/bacterial cells-containing seasoning at 1/10000th to 1/10th of the weight of the bacterial cells improves the effect of the enzymatic reaction on the thickness-enhancing effect.

TABLE 13

| | Enzyme amount | | |
|---|---|---|---|
| | (with respect | | |
| | to bacterial | Thick- | |
| Sample | cell weight) | ness | Comment |
| NC(without | — | 0 | |
| bacterial cells) | | | |
| PC (0.2% | — | 100 | |
| untreated seasoning) | | | |
| Lysozyme-treated | 1/10000 | 120 | a little stronger |
| seasoning | | | thickness than PC |
| Lysozyme-treated | 1/1000 | 150 | stronger thickness than |
| seasoning | | | PC |
| Lysozyme-treated | 1/100 | 170 | stronger thickness than |
| seasoning | | | PC |
| Lysozyme-treated | 1/50 | 170 | stronger thickness than |
| seasoning | | | PC |
| Lysozyme-treated | 1/10 | 170 | stronger thickness than |
| seasoning | | | PC, having sweetness, |
| | | | slight off-flavor |

### Example 10: Effect of Lysozyme Treatment of Corynebacterium casei Fermentation Broth/Bacterial Cells-Containing Seasoning on the Pungency Enhancing Effect

A suspension was obtained by mixing 0.4 g of the C. casei AJ111891-derived mutant strain fermentation broth/bacterial cells-containing seasoning (6.0% cell content) obtained in Example 3 with 19.0 g of distilled water. Lysozyme (BIOCON (JAPAN) LTD., derived from egg white) was added to the suspension at a weight of 1/10000th to 1/10th of the cell weight. Distilled water was added as necessary to bring the total weight of the enzyme reaction solution to 20 g. After the addition of the enzyme solution, the reaction was carried out at 50° C. for 6 h. After the reaction, heating was carried out at 100° C. for 10 min and the solution was ice-cooled to obtan enzyme reaction solution of seasoning. Samples for sensory evaluation were obtained by mixing 10 g of the reaction solution with 90 g of curry sauce (17.2 g of curry roux manufactured by House Foods Corp. mixed with 400 g of hot water). The sample mixed with 10 g of distilled water and 90 g of curry sauce was used as the negative control (NC). A positive control (PC) was obtained by mixing 10 g of a 2% suspension of enzyme-untreated C. casei AJ111891-derived mutant strain fermentation broth/bacterial cells-containing seasoning with 90 g of curry sauce. The sensory evaluation was carried out by a joint rating method by two expert panel members. The pungency intensity of the negative control was given a score of 0, while the pungency intensity when the enzyme-untreated seasoning was added was given a score of 100.

The results are shown in Table 14. The reaction solution obtained by adding lysozyme at 1/1000th to 1/10th of the cell weight showed a stronger pungency-enhancing effect than the enzyme-untreated seasoning. On the other hand, the reaction solution obtained by adding lysozyme at 1/10000th of the cell weight showed a pungency-enhancing effect comparable to that of the enzyme-untreated seasoning. These results indicate that, under the present conditions, the pungency-enhancing effect is improved by adding lysozyme to the seasoning at 1/1000 to 1/10th of the cell weight by weight in the enzymatic reaction.

TABLE 14

| | Enzyme amount | | |
|---|---|---|---|
| | (with respect | | |
| | to bacterial | Pun- | |
| Sample | cell weight) | gency | Comment |
| NC(without | — | 0 | |
| bacterial cells) | | | |
| PC (0.2% | — | 100 | |
| untreated seasoning) | | | |
| Lysozyme-treated | 1/10000 | 100 | equivalent to PC |
| seasoning | | | |
| Lysozyme-treated | 1/1000 | 120 | slightly stronger |
| seasoning | | | pungency than PC |
| Lysozyme-treated | 1/100 | 145 | a little stronger |
| seasoning | | | pungency than PC |
| | | | (having perceptible |
| | | | effect) |
| Lysozyme-treated | 1/50 | 170 | stronger pungency than |
| seasoning | | | PC |
| Lysozyme-treated | 1/10 | 170 | stronger pungency than |
| seasoning | | | PC |

### Example 11: Effect of Enzyme Treatment of Corynebacterium casei Fermentation Broth/Bacterial Cells-Containing Seasoning on Thickness-Enhancing Effect

The enzyme reaction solution of the seasoning obtained in Example 10 was used to investigate the thickness-enhancing effect in a cheese sauce system. Samples for sensory evaluation were obtained by mixing 10 g of the obtained reaction solution with 90 g of cheese sauce Mizkan, RAGU Four Cheese). A negative control (NC) was also obtained by mixing 10 g of distilled water with 90 g of cheese sauce. The positive control (PC) was made by mixing 10 g of a 2% suspension of enzyme-untreated C. casei AJ111891-derived mutant strain fermentation broth/bacterial cells-containing seasoning with 90 g of cheese sauce. The sensory evaluation was carried out by a joint rating method by two expert panel members. The thickness intensity of the negative control was given a score of 0 and that when the enzyme-untreated seasoning was added was given a score of 100.

The results are shown in Table 15. The reaction solutions obtained by adding lysozyme at 1/1000th to 1/10th of the weight of the bacterial cells showed a stronger thickness-enhancing effect than the enzyme-untreated seasonings. On the other hand, the reaction solution obtained by adding lysozyme at 1/10000th of the cell weight showed a similar thickness-enhancing effect as the enzyme-untreated seasoning. These results indicate that, under the present conditions, the addition of lysozyme to the Corynebacterium casei fermentation broth/bacterial cells-containing seasoning at 1/1000th to 1/10th of the weight of the bacterial cells improves the thickness-enhancing effect in the enzymatic reaction.

TABLE 15

| | Enzyme amount | | |
|---|---|---|---|
| | (with respect | | |
| | to bacterial | Thick- | |
| Sample | cell weight) | ness | Comment |
| NC(without | — | 0 | |
| bacterial cells) | | | |
| PC (0.2% | — | 100 | |
| untreated | | | |
| seasoning) | | | |
| Lysozyme-treated | 1/10000 | 100 | equivalent to PC |
| seasoning | | | |
| Lysozyme-treated | 1/1000 | 130 | stronger thickness than |
| seasoning | | | PC |
| Lysozyme-treated | 1/100 | 140 | stronger thickness than |
| seasoning | | | PC, with umami, |
| | | | sweetness |
| Lysozyme-treated | 1/50 | 150 | stronger thickness than |
| seasoning | | | PC, with bad balance |
| Lysozyme-treated | 1/10 | 150 | stronger thickness than |
| seasoning | | | PC, with bad balance |

### Example 12: Effect of Enzyme Treatment of Corynebacterium casei Fermentation Broth/Bacterial Cells-Containing Seasoning on the Quality of Thickness (Cheese Sauce System)

The enzyme reaction solution of the seasoning obtained in Example 10 by adding lysozyme at 1/100th of the weight of the bacterial cells was used to evaluate the effect of the thickness-enhancing effect on the initial to aftertaste in the cheese sauce system. A sample for sensory evaluation was obtained by mixing 10 g of the reaction solution with 90 g of cheese sauce (“Classic Alfredo” from Heinz). A negative control (NC) was also obtained by mixing 10 g of distilled water with 90 g of cheese sauce. The positive control (PC) was made by mixing 10 g of a 2% suspension of enzyme-untreated C. casei AJ111891-derived mutant strain fermentation broth/bacterial cells-containing seasoning with 90 g of cheese sauce. The sensory evaluation was carried out by a consensus rating method by a panel of five experts. The thickness intensity of the negative control was given a score of 0, while the thickness intensity when the enzyme-untreated bacterial cells were added was given a score of 100.

The results are shown in Table 16. The reaction solution obtained by adding lysozyme at 1/100th of the weight of the bacterial cells showed a stronger thickness-enhancing effect than the enzyme-untreated seasoning. The reaction solution obtained by adding lysozyme at 1/100th of the weight of the bacterium showed a particularly pronounced effect in enhancing the initial sensation of thickness.

TABLE 16

| | Enzyme amount | | Middle- | |
|---|---|---|---|---|
| | (with respect | Pre- | and post- | Total |
| | to bacterial | thick- | thick- | thick- |
| Sample | cell weight) | ness | ness | ness |
| PC (0.2% untreated | — | 100 | 100 | 100 |
| seasoning) | | | | |
| Lysozyme-treated | 1/100 | 250 | 130 | 160 |
| seasoning | | | | |

### Example 13: Effect of Enzyme Treatment of Corynebacterium casei Fermentation Broth/Bacterial Cells-Containing Seasoning on the Thickness-Enhancing Effect Perceived in the Initial to Aftertaste (Beef Consommé-Based)

The enzyme reaction solution of the seasoning obtained in Example 10, which was obtained by adding and reacting with lysozyme at 1/100th of the weight of the bacterial cells, was used to evaluate the thickness-enhancing effect perceived in the initial to aftertaste in the beef consommé system. A sample for sensory evaluation was obtained by mixing 10 g of the reaction solution with 90 g of beef consommé (manufactured by Nestlé, “Maggi Additive-free Beef Consommé” prepared for Direction Chicken). A negative control (NC) was also obtained by mixing 10 g of distilled water with 90 g of beef consommé. The positive control (PC) was made by mixing 10 g of a 2% suspension of enzyme-untreated C. casei AJ111891-derived mutant strain fermentation broth/bacterial cells-containing seasoning with 90 g of cheese sauce. The sensory evaluation was carried out by a consensus rating method by a panel of five experts. The thickness intensity of the negative control was given a score of 0, while the thickness intensity when the enzyme-untreated bacterial cells were added was given a score of 100.

The results are shown in Table 17. The reaction solution obtained by adding lysozyme to 1/100th of the weight of the bacterium showed a stronger thickness-enhancing effect than the enzyme-untreated seasoning. In the sample obtained by adding lysozyme to 1/100th of the weight of the bacterial cells, the thickness-enhancing effect perceived initially was particularly pronounced.

TABLE 17

| | Enzyme amount | | Middle- | |
|---|---|---|---|---|
| | (with respect | Pre- | and post- | Total |
| | to bacterial | thick- | thick- | thick- |
| Sample | cell weight) | ness | ness | ness |
| PC (0.2% untreated | — | 100 | 100 | 100 |
| seasoning) | | | | |
| Lysozyme-treated | 1/100 | 250 | 130 | 160 |
| seasoning | | | | |

### Example 14: Effect of Various Enzyme Treatments of Corynebacterium casei Fermentation Broth/Bacterial Cells-Containing Seasoning on the Pungency-Enhancing Effect

A suspension was obtained by mixing 0.2 g of the C. casei AJ111891-derived mutant strain fermentation broth/bacterial cells-containing seasoning (6.0% cell content) obtained in Example 3 with 9.5 g of distilled water. To this suspension, lysozyme (BIOCON (JAPAN) LTD., derived from egg white), papain (Amano Enzyme), cellulase (Amano Enzyme), hemicellulase (Amano Enzyme), nuclease (Amano Enzyme) or lipase (Amano Enzyme) were added at an amount of one tenth of the weight of the bacterial cells. Distilled water was added as appropriate to bring the total weight of the enzyme reaction solution to 10 g. After the addition of the enzyme solution, the reaction was carried out at 45° C. for 4 h. After the reaction, heating was carried out at 100° C. for 10 min and the solution was ice-cooled to obtan enzyme reaction solution of seasoning. A sample for sensory evaluation was obtained by mixing 5 g of the reaction solution with 45 g of curry sauce (17.2 g of curry roux manufactured by House Foods Corp. mixed with 400 g of hot water). The sample mixed with 5 g of distilled water and 45 g of curry sauce was used as the negative control (NC). A positive control (PC) was obtained by mixing 5 g of a 2% suspension of enzyme-untreated C. casei AJ111891-derived mutant strain fermentation broth/bacterial cells-containing seasoning with 45 g of curry sauce. The sensory evaluation was carried out by a joint rating method by two expert panel members. The pungency intensity of the negative control was given a score of 0, while the pungency intensity when the enzyme-untreated seasoning was added was given a score of 100.

The results are shown in Table 18. The reaction solution obtained by adding lysozyme at 1/10th of the cell weight showed a stronger pungency-enhancing effect than the enzyme-untreated seasoning. On the other hand, the reaction solutions obtained by adding papain, cellulase, hemicellulase, nuclease or lipase all showed a pungency-enhancing effect comparable to that of the enzyme-untreated seasoning (positive control: PC). These results indicate that the enhancement of the pungency enhancing effect is specifically obtained by lysozyme treatment.

TABLE 18

| | Enzyme amount | | |
|---|---|---|---|
| | (with respect | | |
| | to bacterial | | |
| Sample | cell weight) | Pungency | Comment |
| NC(without bacterial cells) | — | 0 | |
| PC (0.2% untreated seasoning) | — | 100 | |
| Lysozyme-treated seasoning | 1/10 | 170 | stronger pungency than PC |
| Papain-treated seasoning | 1/10 | 100 | almost equivalent to PC |
| Cellulase-treated seasoning | 1/10 | 100 | almost equivalent to PC |
| Hemicellulase-treated seasoning | 1/10 | 100 | almost equivalent to PC |
| Nuclease-treated seasoning | 1/10 | 100 | almost equivalent to PC |
| Lipase-treated seasoning | 1/10 | 100 | almost equivalent to PC |

### Example 15: Effect of Various Enzyme Treatments of Corynebacterium casei Fermentation Broth/Bacterial Cells-Containing Seasoning on Thickness-Enhancing Effect

The enzyme reaction solution of the seasoning obtained in Example 14 was used to investigate the thickness-enhancing effect in a cheese sauce system. A sample for sensory evaluation was obtained by mixing 2 g of the reaction solution obtained with 18 g of cheese sauce (“Alfredo” manufactured by Kraft Heinz). A negative control (NC) was also obtained by mixing 2 g of distilled water with 18 g of cheese sauce. The positive control (PC) was made by mixing 2 g of a 2% suspension of enzyme-untreated C. casei AJ111891-derived mutant strain fermentation broth/bacterial cells-containing seasoning with 18 g of cheese sauce. The sensory evaluation was carried out by a joint rating method by two expert panel members. The thickness intensity of the negative control was given a score of 0 and that when the enzyme-untreated seasoning was added was given a score of 100.

The results are shown in Table 19. The reaction solution obtained by adding lysozyme at 1/10th of the weight of the bacterial cells showed a stronger thickness-enhancing effect than the enzyme-untreated seasoning. On the other hand, the reaction solutions obtained by adding papain, cellulase, hemicellulase, nuclease or lipase treatment all showed similar thickness-enhancing effects to the enzyme-untreated seasoning. These results indicate that the enhanced thickness-enhancing effect is specifically obtained by lysozyme treatment.

TABLE 19

| | Enzyme amount | | |
|---|---|---|---|
| | (with respect | | |
| | to bacterial | | |
| Sample | cell weight) | Thickness | Comment |
| NC(without bacterial cells) | — | 0 | |
| PC (0.2% untreated seasoning) | — | 100 | |
| Lysozyme-treated seasoning | 1/10 | 150 | stronger thickness than PC |
| Papain-treated seasoning | 1/10 | 100 | almost equivalent to PC |
| Cellulase-treated seasoning | 1/10 | 100 | almost equivalent to PC |
| Hemicellulase-treated seasoning | 1/10 | 100 | almost equivalent to PC |
| Nuclease-treated seasoning | 1/10 | 100 | almost equivalent to PC |
| Lipase-treated seasoning | 1/10 | 100 | almost equivalent to PC |

### Example 16: Effect of Solubilisation

A suspension was obtained by mixing 0.2 g of dried bacterial cells of the Corynebacterium casei AJ111891-derived mutant strain obtained in Example 2 with 19.0 g of distilled water. To this suspension, Lysozyme (from egg white, BIOCON (JAPAN) LTD.) or papain (Amano Enzyme) was added to 1/1000th to 1/10th of the weight of the bacterial cells. Distilled water was added as appropriate to bring the total weight of the enzyme reaction solution to 20 g. After the addition of the enzyme solution, the reaction was carried out at 45° C. for 4 h. After the reaction, heating was carried out at 100° C. for 10 min, ice-cooled and centrifuged at 2500 rpm for 15 min to obtain the supernatant fraction. The obtained supernatant fraction of 10 g was mixed with 90 g of curry sauce (17.2 g of curry roux manufactured by House Foods Corp. mixed with 400 g of hot water) to obtain a sample for sensory evaluation. The supernatant fraction was obtained in the same way from the suspension of the bacterium before enzyme treatment, and 10 g of the supernatant fraction obtained was mixed with curry sauce to obtain e-NC. A mixture of 10 g distilled water and 90 g curry sauce was used as the negative control (NC). The positive control (PC) was obtained by mixing 10 g of 1% suspension of enzyme-untreated bacterial cells with 90 g of curry sauce. The sensory evaluation was carried out by a joint rating method by two expert panel members. The pungency intensity of the negative control was scored 0 and the pungency intensity when the enzyme-untreated bacterial cells were added was scored 100. It was separately confirmed that the pungency of mashed potatoes containing 0.2% by weight of chilli pepper was comparable to that of mashed potatoes containing 0.4% by weight of chilli pepper when 0.1% of the enzyme-untreated bacterial cells were added to the mashed potatoes containing 0.2% by weight of chilli pepper.

The results are shown in Table 20. The supernatant fraction of the reaction solution obtained by adding lysozyme at 1/10000 to 1/10th of the cell weight also showed a pungency-enhancing effect. On the other hand, the supernatant fractions of the reaction solution obtained by adding papain all showed the same pungency-enhancing effect as the negative control. These results indicate that under the present conditions, the supernatant obtained from the enzymatic reaction of Corynebacterium casei with lysozyme at a concentration of 1/10000 to 1/10th of the cell weight also showed a pungency-enhancing effect.

TABLE 20

| | Enzyme amount | | |
|---|---|---|---|
| | (with respect | | |
| | to bacterial | | |
| Sample | cell weight) | Pungency | Comment |
| NC(without bacterial cells) | — | 0 | |
| PC (0.1% untreated bacterial cells) | — | 100 | |
| e-NC (supernatant of untreated | — | 10 | a little stronger pre- and |
| bacterial cells) | | | middle-thickness than PC |
| Lysozyme-treated supernatant | 1/1000 | 100 | stronger pungency than e-NC |
| Lysozyme-treated supernatant | 1/100 | 150 | stronger pungency than e-NC |
| Lysozyme-treated supernatant | 1/50 | 180 | stronger pungency than e-NC |
| Lysozyme-treated supernatant | 1/10 | 150 | sweetness and off-flavor were |
| | | | imparted |
| Papain-treated supernatant | 1/1000 | 130 | equivalent to e-NC |
| Papain-treated supernatant | 1/100 | 10 | equivalent thickness to e-NC, |
| | | | having umami |
| Papain-treated supernatant | 1/50 | 10 | equivalent thickness to e-NC, |
| | | | having umami |
| Papain-treated supernatant | 1/10 | 10 | equivalent thickness to e-NC, |
| | | | off-flavor was imparted |

### Example 17: Effect of Chitinase Treatment on the Pungency-Enhancing Effect of Corynebacterium casei Fermentation Broth/Bacterial Cells-Containing Seasoning

A suspension was obtained by mixing 0.2 g of the C. casei AJ111891-derived mutant strain fermentation broth/bacterial cells-containing seasoning (6.0% cell content) obtained in Example 3 with 9.5 g of distilled water. Chitinase (NAGASE & CO., LTD) was added to this suspension at an amount of one tenth of the weight of the bacterial cells. Distilled water was added as necessary to bring the total weight of the enzyme reaction solution to 10 g. After the addition of the enzyme solution, the reaction was carried out at 45° C. for 4 h. After the reaction, heating was carried out at 100° C. for 10 min and the solution was ice-cooled to obtan enzyme reaction solution of seasoning. A sample for sensory evaluation was obtained by mixing 5 g of the reaction solution with 45 g of curry sauce (17.2 g of curry roux manufactured by House Foods Corp. mixed with 400 g of hot water). The sample mixed with 5 g of distilled water and 45 g of curry sauce was used as the negative control (NC). A positive control (PC) was obtained by mixing 5 g of a 2% suspension of seasoning without enzyme treatment with 45 g of curry sauce. Sensory evaluation was carried out by two expert panel members using the rating method. The pungency intensity of the negative control was scored 0 and the pungency intensity when the enzyme-untreated seasoning was added was given a score of 100.

The results are shown in Table 21. The reaction solutions obtained by adding lysozyme or chitinase at 1/10th of the weight of the bacterial cells showed a stronger pungency-enhancing effect compared to the enzyme-untreated seasoning. The pungency-enhancing effect was particularly pronounced in the reaction solution obtained by adding lysozyme at 1/10th of the cell weight. These results indicate that not only lysozyme treatment but also chitinase treatment improves the pungency-enhancing effect.

TABLE 21

| | Enzyme amount | | |
|---|---|---|---|
| | (with respect | | |
| | to bacterial | | |
| Sample | cell weight) | Pungency | Comment |
| NC(without | — | 0 | |
| bacterial cells) | | | |
| PC (0.2% | — | 100 | |
| untreated seasoning) | | | |
| Lysozyme-treated | 1/10 | 170 | stronger pungency |
| seasoning | | | than PC |
| Chitinase-treated | 1/10 | 120 | a little stronger |
| seasoning | | | pungency than PC |

### Example 18: Effect of Chitinase Treatment of Corynebacterium casei Fermentation Broth/Bacterial Cells-Containing Seasoning on Thickness-Enhancing Effect

The enzyme reaction solution of the seasoning obtained in Example 14 was used to investigate the thickness-enhancing effect in a cheese sauce system. A sample for sensory evaluation was obtained by mixing 2 g of the reaction solution with 18 g of cheese sauce (“Alfredo” manufactured by Kraft Heinz). The sample mixed with 2 g of distilled water and 18 g of cheese sauce was used as the negative control (NC). The positive control (PC) was made by mixing 2 g of a 2% suspension of seasoning without enzyme treatment with 18 g of cheese sauce. Sensory evaluation was carried out by two expert panel members using the rating method. The thickness intensity of the negative control was given a score of 0, while the thickness intensity when the enzyme-untreated seasoning was added was given a score of 100.

The results are shown in Table 22. The reaction solutions obtained by adding lysozyme or chitinase at 1/10th of the weight of the bacterial cells showed a strong thickness-enhancing effect in comparison with the seasonings without enzyme treatment. The effect of enhancing thickness was particularly pronounced in the reaction solution obtained by adding lysozyme to 1/10th of the weight of the bacterial cells. These results indicate that not only lysozyme treatment but also chitinase treatment improves the thickness-enhancing effect.

TABLE 22

| | Enzyme amount | | |
|---|---|---|---|
| | (with respect | | |
| | to bacterial | | |
| Sample | cell weight) | Thickness | Comment |
| NC(without | — | 0 | |
| bacterial cells) | | | |
| PC (0.2% untreated | — | 100 | |
| seasoning) | | | |
| Lysozyme-treated | 1/10 | 150 | stronger thickness |
| seasoning | | | than PC |
| Chitinase-treated | 1/10 | 110 | a little stronger |
| seasoning | | | thickness than PC |

### Example 19: Preparation of Dried Cells of Corynebacterium flavescens

Corynebacterium flavescens NBRC 14136 strain was inoculated into 300 mL-volume conical flasks with baffles filled with 100 mL of the media listed in Table 23 and incubated at 30° C. for 1 day with shaking. The resulting culture medium was sterilised at 120° C. for 20 min and the culture medium components were removed by centrifugation to obtain a cell pellet. The bacterial cells were further washed by adding the same amount of water as the culture medium and the supernatant was removed by centrifugation, which was repeated twice. After washing, the bacterium pellets were frozen at −80° C. and the water was removed by a freeze-dryer to obtain dried cells of Corynebacterium flavescens NBRC 14136 strain.

TABLE 23

| Component | Final concentration | |
|---|---|---|
| Potato peotone | 10 | g/L |
| Yeast Extract | 20 | g/L |
| Glucose | 10 | g/L |
| NaCl | 5 | g/L |

### Example 20: Effect of Lysozyme Treatment of the Cells of Corynebacterium flavescens on Pungency-Enhancing Effect

A suspension was obtained by mixing 0.2 g of dried cells of C. flavescens NBRC 14136 strain obtained in Example 19 with 19.0 g of distilled water. To this suspension, Lysozyme (BIOCON (JAPAN) LTD., derived from egg white) was added at 1/100th and 1/100th of the weight of the bacterial cells. Distilled water was added as appropriate to bring the total weight of the enzyme reaction solution to 20 g. After the addition of the enzyme solution, the reaction was carried out at 45° C. for 4 h. After the reaction, heating was carried out at 100° C. for 10 min and the solution was ice-cooled to obtan enzyme reaction solution of the bacterial cells. A sample for sensory evaluation was obtained by mixing 10 g of the reaction solution with 90 g of curry sauce (17.2 g of curry roux manufactured by House Foods Corp. mixed with 400 g of hot water). The sample mixed with 10 g of distilled water and 90 g of curry sauce was used as the negative control (NC). A positive control (PC) was obtained by mixing 10 g of a 1% suspension of the enzyme-untreated cells of C. casei AJ111891-derived mutant strain obtained in Example 2 with 90 g of curry sauce. In addition, 10 g of a 1% suspension of the enzyme-untreated cells of C. flavescens NBRC 14136 strain mixed with 90 g of curry sauce was also sensory evaluated. The sensory evaluation was carried out by a panel of three experts using a rating system. The pungency intensity of the negative control was scored 0 and that of the positive control was given a score of 100.

The results are shown in Table 24. Enzyme-untreated C. flavescens bacterium showed the pungency enhancing effect comparable to enzyme-untreated C. casei bacterium. The reaction solutions obtained by adding lysozyme at 1/1000th and 1/100th of the cell weight of C. flavescens showed a stronger thickness enhancing effect than the enzyme untreated cells of C. flavescens and C. casei. These results indicate that, under the present conditions, the pungency-enhancing effect was improved by adding lysozyme to C. flavescens at 1/1000th to 1/100th of the cell weight in the enzymatic reaction.

TABLE 24

| | Enzyme amount | | |
|---|---|---|---|
| | (with respect | | |
| | to bacterial | | |
| Sample | cell weight) | Pungency | Comment |
| NC (without bacterial cells) | — | 0 | |
| PC (0.1% untreated bacterial | — | 100 | |
| cells (C. casei)) | | | |
| 0.1% untreated bacterial cells | — | 100 | lagging pungency than PC, almost |
| (C. flavescens) | | | equivalent pungency to PC |
| Lysozyme-treated bacterial | 1/1000 | 180 | lagging pungency than PC, stronger |
| cells (C. flavescens) | | | pungency than PC |
| Lysozyme-treated bacterial | 1/100 | 180 | lagging pungency than PC, stronger |
| cells (C. flavescens) | | | pungency than PC |

### Example 21: Preparation of Dried Cells of Corynebacterium ammoniagenes

Corynebacterium ammoniagenes ATCC21280 strain was cultured by jar culture using the media listed in Table 25. A 1 L-volume fermenter was used for jar culture. The culture temperature was maintained at a constant 34° C., the culture pH was controlled to 6.8 with ammonia gas and the dissolved oxygen concentration was controlled by agitation control to be at least 23% as a relative value with the saturated dissolved oxygen concentration as 100%. The obtained culture medium was sterilised at 70° C. for 10 min and the culture medium components were removed by centrifugation to obtain a cell pellet. The bacterial cells were further washed by adding the same amount of water as the culture medium and the supernatant was removed by centrifugation, which was repeated twice. After washing, the cell pellet was frozen at −80° C. and the water was removed by a freeze-dryer to obtain dried cells of Corynebacterium ammoniagenes ATCC21280 strain. The dried cells were heated at 105° C. for 4 hours and the solids content in the dried cells was determined by measuring the loss on drying.

TABLE 25

| Component | Final concentration | |
|---|---|---|
| Glucose | 60 | g/L |
| MgSO₄•7H₂O | 0.90 | g/L |
| KH₂PO₄ | 3.5 | g/L |
| FeSO₄•7H₂O | 10 | mg/L |
| Disodium succinate•6H₂O | 2.0 | g/L |
| Sodium L(+)-ascorbate | 8.6 | mg/L |
| Thiamin Hydrochloride | 23 | mg/L |
| VB12 | 4.0 | μ/L |
| Yeast Extract | 15 | g/L |
| Plant protein hydrolysate | 900 mg/L (in terms of total nitrogen) | |
| Edible oil | 1.5 | mL/L |

### Example 22: Effect of Lysozyme Treatment of the Cells of Corynebacterium ammnoniageses on Pungency-Enhancing Effect

A suspension was obtained by mixing 0.2 g (solid content) of dried cells of C. ammoniagenes ATCC21280 strain obtained in Example 21 with 19.0 g of distilled water. To this suspension, Lysozyme (BIOCON (JAPAN) LTD., derived from egg white) was added at 1/100th and 1/1000th of the weight of the bacterial cells. Distilled water was added as required to bring the total weight of the enzyme reaction solution to 20 g. After the addition of the enzyme solution, the reaction was carried out at 45° C. for 4 h. After the reaction, heating was carried out at 100° C. for 10 min and the solution was ice-cooled to obtan enzyme reaction solution of the bacterial cells. A sample for sensory evaluation was obtained by mixing 10 g of the reaction solution with 90 g of curry sauce (17.2 g of curry roux manufactured by House Foods Corp. mixed with 400 g of hot water). The mixture of 10 g distilled water and 90 g curry sauce was used as the negative control (NC). The positive control (PC) was made by mixing 10 g of a 1% suspension of the enzyme-untreated C. casei AJ111891-derived mutant strain obtained in Example 2 with 90 g of curry sauce. A sensory evaluation was also carried out on 10 g of a 1% suspension of the enzyme-untreated cells of C. ammoniagenes ATCC21280 strain mixed with 90 g of curry sauce. The sensory evaluation was carried out by a panel of three experts using a rating system. The pungency intensity of the negative control was scored 0 and that of the positive control was scored 100.

The results are shown in Table 26. The enzyme-untreated cells of C. ammoniagenes did not show as the strong pungencyenhancing effect as the enzyme-untreated cells of C. casei. On the other hand, the reaction solutions obtained by adding lysozyme at 1/100th and 1/1000th of the cell weight of C. ammnoniageses showed a stronger pungency-enhancing effect than the enzyme-untreated cells of C. ammnoniageses and C. casei. These results indicate that, under the present conditions, the pungency-enhancing effect was enhanced by adding lysozyme to C. ammnoniageses at 1/1000th to 1/100th of the cell weight in the enzymatic reaction.

TABLE 26

| | Enzyme amount | | |
|---|---|---|---|
| | (with respect | | |
| | to bacterial | | |
| Sample | cell weight) | Pungency | Comment |
| NC (without bacterial cells) | — | 2 | |
| PC (0.01% untreated | — | 100 | middle- and post-pungency were |
| bacterial cells (C. casei)) | | | enhanced by about twice as much |
| 0.01% untreated bacterial | — | 75 | lagging pungency than PC, weaker |
| cells (C. ammoniagenes) | | | pungency than PC |
| Lysozyme-treated bacterial | 1/1000 | 163 | lagging pungency than PC, stronger |
| cells (C. ammoniagenes) | | | pungency than PC |
| Lysozyme-treated bacterial | 1/100 | 173 | lagging pungency than PC, stronger |
| cells (C. ammoniagenes) | | | pungency than PC |

### Example 23: Preparation of Dried Cells of Corynebacterium glutamicum

Corynebacterium glutamicum ATCC13869 strain was cultured by jar culture using the media listed in Table 25. A 1 L-volume fermenter was used for jar culture. The culture temperature was maintained at a constant 31.5° C., the culture pH was controlled to 6.8 with ammonia gas and the dissolved oxygen concentration was controlled by agitation control to be at least 23% as a relative value with the saturated dissolved oxygen concentration as 100%. The culture medium was sterilised at 70° C. for 10 min, and the culture medium components were removed by centrifugation to obtain a cell pellet. The bacterial cells were further washed by adding the same amount of water as the culture medium and the supernatant was removed by centrifugation, which was repeated twice. After washing, the cell pellet was frozen at −80° C. and the water was removed by a freeze-dryer to obtain dried cells of Corynebacterium glutamicum ATCC13869 strain. The dried bacterial cells were heated at 105° C. for 4 hours and the solids content in the dried bacterial cells was determined by measuring the loss on drying.

### Example 24: Effect of Lysozyme Treatment of Cells of Corynebacterium glutamicum on the Pungency-Enhancing Effect

A suspension was obtained by mixing 0.2 g (solid content) of dried bacterium of C. glutamicum ATCC13869 strain obtained in Example 23 with 19.0 g of distilled water. To this suspension, Lysozyme (BIOCON (JAPAN) LTD., derived from egg white) was added to reach 1/100th of the weight of the bacterial cells. Distilled water was added as necessary to bring the total weight of the enzyme reaction solution to 20 g. After the addition of the enzyme solution, the reaction was carried out at 45° C. for 4 h. After the reaction, heating was carried out at 100° C. for 10 min and the solution was ice-cooled to obtan enzyme reaction solution of the bacterial cells. A sample for sensory evaluation was obtained by mixing 1 g of the reaction solution obtained and 0.2 g of commercially available fermented umami seasoning (containing 20% glutamic acid, manufactured by CJ) with 99 g of curry sauce (17.2 g of curry roux manufactured by House Foods Corp. mixed with 400 g of hot water). The sample mixed with 1 g of distilled water and 99 g of curry sauce was used as the negative control (NC). The positive control (PC) was a mixture of 1 g of a 1% suspension of the enzyme-untreated cells of C. casei AJ111891-derived mutant obtained in Example 2, 0.2 g of the aforementioned fermented umami seasoning and 99 g of curry sauce. A mixture of 1 g of a 1% suspension of enzyme-untreated cells of Corynebacterium glutamicum, 0.2 g of the aforementioned fermented umami seasoning and 99 g of curry sauce was also sensory evaluated. The sensory evaluation was carried out by a panel of three experts using the rating method. The pungency intensity of the negative control was scored 0 and that of the positive control was scored 100.

The results are shown in Table 27. The enzyme-untreated cells of C. glutamicum did not show as the strong pungency-enhancing effect as the enzyme-untreated cells of C. casei. On the other hand, the reaction solution obtained by adding lysozyme at 1/100th of the cell weight of C. glutamicum showed a stronger pungency-enhancing effect than the enzyme-untreated cells of C. glutamicum. These results indicate that, under the present conditions, the pungency-enhancing effect is improved by adding lysozyme to the cells of C. glutamicum at 1/100th of the cell weight in the enzymatic reaction.

TABLE 27

| | Enzyme amount | | |
|---|---|---|---|
| | (with respect | | |
| | to bacterial | | |
| Sample | cell weight) | Pungency | Comment |
| NC (without bacterial cells) | — | 0 | |
| PC (0.01% untreated bacterial | — | 100 | |
| cells (C. casei)) | | | |
| 0.01% untreated bacterial cells | — | 60 | lagging pungency than PC, weaker |
| (C. ammoniagenes) | | | pungency than PC |
| Lysozyme-treated bacterial | 1/100 | 95 | lagging pungency than PC. |
| cells (C. ammoniagenes) | | | equivalent to PC |

### Example 25: Preparation of Dried Cells of Corynebacterium stationis

Corynebacterium stationis ATCC6872 strain was cultured by jar culture using the media listed in Table 28. A 1 L-volume fermenter was used for jar culture. The culture temperature was maintained at a constant 34° C., the culture pH was maintained at 6.8 with ammonia gas and the dissolved oxygen concentration was controlled by agitation control to be at least 23% relative to the saturated dissolved oxygen concentration of 100%. The culture medium was sterilised at 80° C. for 10 min, and the culture medium components were removed by centrifugation to obtain a cell pellet. The bacterium were further washed by adding the same amount of water as the culture medium and the supernatant was removed by centrifugation, which was repeated twice. After washing, the cell pellet was frozen at −80° C. and the water was removed by a freeze-dryer to obtain dried bacterial cells of Corynebacterium stationis ATCC6872 strain. The dried bacterial cells were heated at 105° C. for 4 hours and the solids content in the dried bacterial cells was determined by measuring the loss on drying.

TABLE 28

| Component | Final concentration | |
|---|---|---|
| Glucose | 60 | g/L |
| MgSO₄•7H₂O | 0.90 | g/L |
| KH₂PO₄ | 3.5 | g/L |
| FeSO•7H₂O | 10 | mg/L |
| Disodium succinate•6H₂O | 2.0 | g/L |
| Sodium L(+)-ascorbate | 8.6 | mg/L |
| Thiamin Hydrochloride | 23 | mg/L |
| VB12 | 4.0 | μ/L |
| Yeast Extract | 15 | g/L |
| Plant protein hydrolysate | 900 mg/L (in terms of total nitrogen) | |
| Edible oil | 7.5 | mL/L |

### Example 26: Effect of Lysozyme Treatment of Cells of Corynebacterium stationis on Pungency-Enhancing Effect

A suspension was obtained by mixing 0.2 g (solid content) of dried bacterial cells of C. stationis ATCC6872 obtained in Example 25 with 19.0 g of distilled water. To this suspension, Lysozyme (BIOCON (JAPAN) LTD., derived from egg white) was added to reach 1/100th of the cell weight. Distilled water was added as necessary to bring the total weight of the enzyme reaction solution to 20 g. After the addition of the enzyme solution, the reaction was carried out at 45° C. for 4 h. After the reaction, heating was carried out at 100° C. for 10 min and the solution was ice-cooled to obtan enzyme reaction solution of the bacterial cells. A sample for sensory evaluation was obtained by mixing 1 g of the reaction solution obtained and 0.2 g of commercially available fermented umami seasoning (containing 20% glutamic acid, manufactured by CJ) with 99 g of curry sauce (17.2 g of curry roux manufactured by House Foods Corp. mixed with 400 g of hot water). The sample mixed with 1 g of distilled water and 99 g of curry sauce was used as the negative control (NC). The positive control (PC) was a mixture of 1 g of a 1% suspension of the enzyme-untreated cells of C. casei AJ111891-derived mutant strain obtained in Example 2, 0.2 g of the aforementioned fermented umami seasoning and 99 g of curry sauce. A mixture of 1 g of a 1% suspension of the enzyme-untreated cells of C. stationis ATCC6872 strain, 0.2 g of the aforementioned fermented umami seasoning and 99 g of curry sauce was also sensory evaluated. The sensory evaluation was carried out by two expert panel members using the rating method. The pungency intensity of the negative control was scored 0 points and that of the positive control was given a score of 100.

The results are shown in Table 29. The enzyme-untreated cells of C. stationis did not show as the strong pungency-enhancing effect as the enzyme-untreated cells of C. casei. On the other hand, the reaction solution obtained by adding lysozyme at 1/100th of the cell weight of C. stationis showed a stronger pungency-enhancing effect than the enzyme-untreated cells of C. stationis and C. casei. These results indicate that, under the present conditions, the pungency-enhancing effect was enhanced by adding lysozyme to Corynebacterium stationis at 1/100th of the cell weight in the enzymatic reaction.

TABLE 29

| | Enzyme amount | | |
|---|---|---|---|
| | (with respect | | |
| | to bacterial | | |
| Sample | cell weight) | Pungency | Comment |
| NC (without bacterial cells) | — | 0 | |
| PC (0.01% untreated | — | 100 | |
| seasoning (C. casei)) | | | |
| 0.01% untreated seasoning | — | 70 | lagging pungency than PC, |
| (C. stationis) | | | weaker pungency than PC |
| Lysozyme-treated seasoning | 1/100 | 130 | stronger pungency than PC |
| (C. stationis) | | | |

### Example 27: Relationship Between the Content of Bound Diaminopimelic Acid and the Effect of Lysozyme Treatment of Cells of Corynebacterium casei on the Pungency-Enhancing Effect

The enzyme reaction solutions of the seasonings obtained in Examples 8 and 10, which were obtained by adding and reacting with lysozyme at 1/10000 to 1/10th of the cell weight, were used to determine the diaminopimelic acid content in the fraction with a molecular weight of 10000 Da or less.

The positive control obtained in Example 10, the reaction solution obtained by adding lysozyme to the enzyme reaction solution of the seasoning obtained in Example 10 in amounts of 1/10000, 1/1000 and 1/10th of the cell weight, and the enzyme reaction solutions of the seasoning obtained in Example 8 obtained by adding lysozyme at 1/10000, 1/1000 and 1/10th of the cell weight were filtered through a microfiltration filter with a pore diameter of 0.45 μm and then filtered through a Vivaspin (registered trademark) 20 (manufactured by Sartorius) centrifugal filtration membrane with an MWCO of 10000 Da at 2800 rpm for 15 min to obtain a fraction with a molecular weight of 10000 Da. The free diaminopimeric acid content was calculated by measuring the diaminopimeric acid content in the fractions obtained. Hydrochloric acid hydrolysis was carried out by adding concentrated hydrochloric acid to the obtained fractions so that the hydrochloric acid concentration was 6 M, followed by heating under reduced pressure at 120° C. for 24 hours. The hydrochloric acid was then distilled off using a decompression concentrator and the sample for analysis was brought up to a predetermined volume. The diaminopimeric acid content in the sample for analysis was determined using LC-MS (high performance liquid chromatography-mass spectrometry). The content of bound diaminopimeric acid was calculated by subtracting the aforementioned free diaminopimeric acid content from the measured diaminopimeric acid content in the sample for analysis.

The results are shown in Table 30. The presence or absence of an “improved pungency-enhancing effect” in Table 30 was judged based on the results of the sensory evaluation in Examples 8 and 10. A pungency intensity of 110 points or more was judged as having an “improved effect of enhancing pungency”, while a pungency intensity of less than 110 points was judged as having “no improved effect of enhancing pungency”. The content of bound diaminopimeric acid in the fraction with a molecular weight of 10000 Da in Table 30 is the content of bound diaminopimeric acid calculated above and converted to the content per 100 g dry weight of the C. casei AJ111891-derived mutant strain fermentation broth/bacterial cells-containing seasoning obtained in Example 3.

As can be seen from Table 30, the content of bound diaminopimetic acid with a molecular weight of 10000 Da or less in the sample determined to have no effect on enhancing pungency was from 17.9 to 19.6 mg per 100 g of solids, whereas the content of bound diaminopimetic acid with a molecular weight of 10000 Da or less in the sample determined to have an effect on enhancing pungency was from 44.6 to 50.9 mg per 100 g of solids. These results indicate that the samples in which the pungency-enhancing effect was improved by lysozyme treatment contained more bound diaminopimeric acid with a molecular weight of less than 10000 Da than those in which the effect were not improved.

TABLE 30

| | | Content of bound diaminopimetic | |
|---|---|---|---|
| | Enzyme amount | acid in the fraction with a molecular | Improving |
| | (with respect | weight of 10000 Da or less per 100 | effect on |
| | to bacterial | g dry weight of sample for sensory | pungency |
| Sample | cell weight) | evaluation | enhancement |
| PC (untreated seasoning | — | 19.6 | No |
| (0.2% C. casei AJ111891- | | | |
| derived mutant strain)) | | | |
| Lysozyme-treated | 1/10000 | 17.9 | No |
| seasoning (C. casei | | | |
| AJ111891-derived mutant | | | |
| strain) | | | |
| Lysozyme-treated | 1/1000 | 44.6 | Yes |
| seasoning (C. casei | | | |
| AJ111891-derived mutant | | | |
| strain) | | | |
| Lysozyme-treated | 1/10 | 50.9 | Yes |
| Seasoning (C. casei | | | |
| AJ111891-derived mutant | | | |
| strain) | | | |
| Lysozyme-treated | 1/10000 | 48.1 | Yes |
| seasoning (C. casei | | | |
| AJ111891 strain) | | | |
| Lysozyme-treated | 1/1000 | 48.1 | Yes |
| seasoning (C. casei | | | |
| AJ111891 strain) | | | |
| Lysozyme-treated | 1/10 | 50.0 | Yes |
| seasoning (C. casei | | | |
| AJ111891 strain) | | | |

What is claimed is:

1. A method for improving a flavor of food product, comprising
a step of adding the following component (A) to an ingredient of a food product:
(A) lysozyme-treated product of cell walls of a gram-positive bacterium.

2. A method for producing a food product with improved flavor, comprising
a step of adding the following component (A) to an ingredient of a food product:
(A) lysozyme-treated product of cell walls of a gram-positive bacterium.

3. The method according to claim 1, wherein the improvement of flavor is an enhancement of spiciness and/or an impartment of kokumi.

4. The method according to claim 1, wherein the improvement of flavor is an enhancement of pungency of spices and/or an impartment of thickness.

5. The method according to claim 1, wherein the component (A) is a lysozyme-treated product of cells of the gram-positive bacterium or fragments thereof.

6. The method according to claim 1, wherein the gram-positive bacterium is a bacterium belonging to the phylum Actinobacteria or Firmicutes.

7. The method according to claim 1, wherein the gram-positive bacterium is a bacterium belonging to the phylum Actinobacteria.

8. The method according to claim 1, wherein the gram-positive bacterium is a coryneform bacterium, a bacterium belonging to the family Bifidobacteriumceae, a bacterium belonging to the family Dermabacteraceae, a bacterium belonging to the family Bacillaceae, a bacterium belonging to the family Enterococcaceae, or a bacterium belonging to the family Lactobacilloae.

9. The method according to claim 1, wherein the gram-positive bacterium is a bacterium of the genus Corynebacterium, a bacterium of the genus Brevibacterium, a bacterium of the genus Bifidobacterium, a bacterium of the genus Brachybacterium, a bacterium of the genus Bacillus, a bacterium of the genus Enterococcus, or a bacterium of the genus Lactobacillus.

10. The method according to claim 1, wherein the gram-positive bacterium is Corynebacterium casei, Corynebacterium flavescens, Corynebacterium ammoniagenes (Corynebacterium stationis), Corynebacterium glutamicum, Brevibacterium casei, Bifidobacterium longum, Brachybacterium alimentarium, Bacillus subtilis, Enterococcus faecalis, Lactobacillus mali, Lactobacillus hilgardii or Lactobacillus brevis.

11. The method according to claim 1, wherein the component (A) is added such that a concentration thereof at the time of eating or drinking is from 0.005 to 2% (w/w) in terms of dry weight of the original cells of the gram-positive bacterium.

12. The method according to claim 1, further comprising
a step of adding the following component (B) to the ingredient of the food product:
(B) one or more components selected from the group consisting of an L-amino acid, a nucleic acid and an organic acid.

13. The method according to claim 12, wherein at least the L-amino acid is added and wherein the L-amino acid is L-glutamic acid.

14. The method according to claim 13, wherein the L-glutamic acid is added such that a concentration thereof at the time of eating or drinking is from 0.01 to 2% (w/w).

15. The method according to claim 12, which is carried out such that a content of the L-glutamic acid in the food product is from 0.1 to 20 parts by weight of the component (A) in terms of dry weight of original cells of the gram-positive bacterium.

16. The method according to claim 1, wherein the food product is a food product containing a spice.

17. The method according to claim 16, wherein a content of the spice in the food product is from 0.01 to 2% (w/w) as a concentration thereof at the time of eating or drinking.

18. The method according to claim 16, which is carried out such that the content of the spice in the food product is from 0.2 to 500 parts by weight of the component (A) in terms of dry weight of original cells of the gram-positive bacterium.

19. The method according to claim 16, wherein the spice is one or more spices selected from the group consisting of a spice of the family Lauraceae, a spice of the family Piperaceae, a spice of the family Lamiaceae, a spice of the family Apiaceae, a spice of the family Solanaceae, a spice of the family Myristicaceae, a spice of the family Alliaceae, a spice of the family Myrtaceae, a spice of the family Schisandraceae, a spice of the family Fabaceae, a spice of the family Polygonaceae, a spice of the family Brassicacceae, a spice of the family Zingiberaceae, and a spice of the family Rutaceae.

20. The method according to claim 16, wherein the spice is a pungent spice.

21. The method according to claim 1, wherein the gram-positive bacterium is a bacterium having an L-glutamic acid-producing ability and has one or more mutations selected from the mutations listed in Table 1
TABLE 1

| | | Pre- | Post- |
|---|---|---|---|
| | Genomic | mutation | mutation |
| No. | position | base | base |
| A-1 | 78,486 | C | T |
| A-2 | 83,592 | G | A |
| A-3 | 87,955 | C | T |
| A-4 | 90,041 | C | T |
| A-5 | 186,221 | C | T |
| A-6 | 193,010 | C | T |
| A-7 | 196,531 | C | T |
| A-8 | 225,429 | C | T |
| A-9 | 297,920 | G | A |
| A-10 | 320,354 | C | T |
| A-11 | 335,878 | C | T |
| A-12 | 341,763 | C | T |
| A-13 | 346,969 | C | T |
| A-14 | 349,856 | C | T |
| A-15 | 356,232 | C | T |
| A-16 | 357,008 | C | T |
| A-17 | 366,674 | G | A |
| A-18 | 369,871 | G | A |
| A-19 | 377,420 | G | A |
| A-20 | 378,652 | G | A |
| A-21 | 432,252 | C | A |
| A-22 | 439,021 | G | A |
| A-23 | 440,764 | G | A |
| A-24 | 454,682 | G | A |
| A-25 | 458,729 | G | A |
| A-26 | 470,562 | G | A |
| A-27 | 471,288 | G | A |
| A-28 | 472,023 | G | A |
| A-29 | 504,885 | G | A |
| A-30 | 505,785 | G | A |
| A-31 | 514,371 | G | A |
| A-32 | 518,684 | G | A |
| A-33 | 521,126 | G | A |
| A-34 | 524,551 | G | A |
| A-35 | 660,841 | C | T |
| A-36 | 732,121 | C | T |
| A-37 | 787,055 | C | T |
| A-38 | 806,047 | C | T |
| A-39 | 872,482 | G | A |
| A-40 | 878,069 | C | T |
| A-41 | 903,037 | C | T |
| A-42 | 922,802 | C | T |
| A-43 | 948,145 | C | T |
| A-44 | 955,819 | C | T |
| A-45 | 968,915 | C | T |
| A-46 | 973,013 | C | T |
| A-47 | 974,797 | C | T |
| A-48 | 994,815 | C | T |
| A-49 | 1,000,498 | C | T |
| A-50 | 1,019,704 | C | T |
| A-51 | 1,049,052 | C | T |
| A-52 | 1,069,322 | C | T |
| A-53 | 1,070,554 | C | T |
| A-54 | 1,131,016 | C | T |
| A-55 | 1,138,639 | C | T |
| A-56 | 1,162,588 | C | T |
| A-57 | 1,193,273 | C | T |
| A-58 | 1,203,146 | C | T |
| A-59 | 1,222,633 | C | T |
| A-60 | 1,226,969 | G | A |
| A-61 | 1,264,895 | G | A |
| A-62 | 1,268,790 | G | A |
| A-63 | 1,279,676 | G | A |
| A-64 | 1,363,909 | T | C |
| A-65 | 1,387,476 | G | A |
| A-66 | 1,401,171 | G | A |
| A-67 | 1,416,228 | C | T |
| A-68 | 1,420,034 | C | T |
| A-69 | 1,447,494 | C | T |
| A-70 | 1,448,318 | C | T |
| A-71 | 1,448,776 | C | T |
| A-72 | 1,451,922 | C | T |
| A-73 | 1,466,961 | C | T |
| A-74 | 1,503,736 | C | T |
| A-75 | 1,504,207 | C | T |
| A-76 | 1,505,998 | C | T |
| A-77 | 1,507,027 | C | T |
| A-78 | 1,544,310 | C | T |
| A-79 | 1,554,973 | C | T |
| A-80 | 1,558,509 | C | T |
| A-81 | 1,562,459 | C | T |
| A-82 | 1,572,716 | C | T |
| A-83 | 1,594,314 | C | T |
| A-84 | 1,602,545 | C | T |
| A-85 | 1,659,808 | C | T |
| A-86 | 1,682,132 | C | T |
| A-87 | 1,689,863 | C | T |
| A-88 | 1,744,963 | C | T |
| A-89 | 1,784,642 | C | T |
| A-90 | 1,814,866 | C | T |
| A-91 | 1,829,145 | C | T |
| A-92 | 1,852,511 | G | A |
| A-93 | 1,861,170 | G | A |
| A-94 | 1,902,133 | G | A |
| A-95 | 1,916,048 | C | T |
| A-96 | 1,917,434 | C | T |
| A-97 | 1,938,271 | C | T |
| A-98 | 1,949,357 | G | T |
| A-99 | 1,954,368 | C | T |
| A-100 | 1,967,997 | C | T |
| A-101 | 1,975,599 | C | T |
| A-102 | 2,141,466 | C | T |
| A-103 | 2,308,064 | C | T |
| A-104 | 2,310,428 | C | T |
| A-105 | 2,354,420 | C | T |
| A-106 | 2,449,270 | T | C |
| A-107 | 2,449,278 | C | A |
| A-108 | 2,449,291 | G | C |
| A-109 | 2,449,318 | G | A |
| A-110 | 2,496,945 | C | T |
| A-111 | 2,505,022 | C | T |
| A-112 | 2,505,285 | C | T |
| A-113 | 2,525,513 | G | A |
| A-114 | 2,565,856 | C | T |
| A-115 | 2,601,306 | G | A |
| A-116 | 2,615,688 | G | A |
| A-117 | 2,650,740 | G | A |
| A-118 | 2,653,259 | G | A |
| A-119 | 2,663,827 | G | A |
| A-120 | 2,667,322 | G | A |
| A-121 | 2,674,077 | G | A |
| A-122 | 2,679,915 | G | A |
| A-123 | 2,686,979 | G | A |
| A-124 | 2,693,950 | C | T |
| A-125 | 2,696,737 | C | T |
| A-126 | 2,706,442 | C | T |
| A-127 | 2,709,469 | C | T |
| A-128 | 2,711,214 | C | T |
| A-129 | 2,714,651 | C | T |
| A-130 | 2,721,339 | G | A |
| A-131 | 2,731,030 | G | A |
| A-132 | 2,746,202 | G | A |
| A-133 | 2,805,389 | C | T |
| A-134 | 2,816,733 | G | A |
| A-135 | 2,827,114 | G | A |
| B-1 | 29,724 | G | A |
| B-2 | 92,869 | G | A |
| B-3 | 116,733 | G | A |
| B-4 | 131,184 | G | A |
| B-5 | 156,247 | G | A |
| B-6 | 177,083 | G | A |
| B-7 | 184,379 | G | A |
| B-8 | 212,586 | G | A |
| B-9 | 282,162 | G | A |
| B-10 | 309,483 | G | A |
| B-11 | 376,164 | C | T |
| B-12 | 440,885 | C | T |
| B-13 | 479,120 | G | A |
| B-14 | 722,430 | G | A |
| B-15 | 745,504 | G | A |
| B-16 | 809,993 | G | A |
| B-17 | 859,643 | G | A |
| B-18 | 923,209 | G | A |
| B-19 | 924,973 | G | A |
| B-20 | 998,893 | C | T |
| B-21 | 1,062,144 | C | T |
| B-22 | 1,095,062 | C | T |
| B-23 | 1,102,484 | C | T |
| B-24 | 1,103,812 | C | T |
| B-25 | 1,105,749 | C | T |
| B-26 | 1,107,561 | C | T |
| B-27 | 1,205,722 | C | T |
| B-28 | 1,233,449 | C | T |
| B-29 | 1,242,484 | C | T |
| B-30 | 1,248,388 | C | T |
| B-31 | 1,249,270 | C | T |
| B-32 | 1,291,377 | C | T |
| B-33 | 1,308,597 | C | T |
| B-34 | 1,329,535 | C | T |
| B-35 | 1,367,486 | C | T |
| B-36 | 1,382,065 | C | T |
| B-37 | 1,403,043 | C | T |
| B-38 | 1,433,914 | C | T |
| B-39 | 1,442,447 | C | T |
| B-40 | 1,501,903 | G | A |
| B-41 | 1,504,744 | C | T |
| B-42 | 1,651,403 | G | A |
| B-43 | 1,695,473 | G | A |
| B-44 | 1,779,939 | G | A |
| B-45 | 1,797,452 | G | A |
| B-46 | 1,801,284 | G | A |
| B-47 | 1,816,679 | G | A |
| B-48 | 1,832,252 | G | A |
| B-49 | 1,843,841 | G | A |
| B-50 | 1,868,285 | G | A |
| B-51 | 1,879,922 | G | A |
| B-52 | 1,892,007 | G | A |
| B-53 | 1,916,016 | G | A |
| B-54 | 1,937,604 | G | A |
| B-55 | 1,947,044 | G | A |
| B-56 | 1,948,411 | G | A |
| B-57 | 1,948,649 | G | A |
| B-58 | 1,967,697 | G | A |
| B-59 | 1,974,137 | G | A |
| B-60 | 2,028,284 | C | T |
| B-61 | 2,050,998 | G | A |
| B-62 | 2,052,708 | G | A |
| B-63 | 2,054,372 | G | A |
| B-64 | 2,065,568 | G | A |
| B-65 | 2,067,167 | G | A |
| B-66 | 2,082,577 | G | A |
| B-67 | 2,121,006 | A | G |
| B-68 | 2,149,369 | C | T |
| B-69 | 2,159,680 | G | A |
| B-70 | 2,380,965 | G | A |
| B-71 | 2,477,728 | G | A |
| B-72 | 2,542,800 | G | A |
| B-73 | 2,570,107 | G | A |
| B-74 | 2,647,383 | G | A |
| B-75 | 2,726,248 | C | T |
| B-76 | 2,825,055 | C | T |
| B-77 | 2,837,078 | C | T |
| B-78 | 2,865,322 | C | T |
| B-79 | 2,872,907 | C | T |
| B-80 | 2,880,351 | C | T |
| B-81 | 2,889,394 | C | T |
| B-82 | 2,906,471 | C | T |
| B-83 | 2,927,044 | C | T |
| B-84 | 2,929,963 | C | T |
| B-85 | 2,940,673 | C | T |
| B-86 | 2,946,285 | C | T |
| B-87 | 2,962,909 | C | T |
| B-88 | 2,975,742 | C | T |
| B-89 | 2,987,052 | C | T |
| B-90 | 3,079,560 | C | T |
| B-91 | 3,083,927 | C | T |
| B-92 | 3,090,163 | C | T |

22. The method according to claim 1, further comprising a step of adding a culture of gram-positive bacterium to the ingredient of the food product.

23. A method for improving a flavor of food product, comprising
a step of adding the following component (A) to an ingredient of a food product:
(A) chitinase-treated product of cell walls of a gram-positive bacterium.

24. A method for producing a food product with improved flavor, comprising
a step of adding the following component (A) to an ingredient of a food product:
(A) chitinase-treated product of cell walls of a gram-positive bacterium.

25. The method according to claim 23, wherein the improvement of flavor is an enhancement of spiciness and/or an impartment of kokumi.

26. The method according to claim 23, wherein the improvement of flavor is an enhancement of pungency of spices and/or an impartment of thickness.

27. The method according to claim 23, wherein the component (A) is a chitinase-treated product of cells of the gram-positive bacterium or fragments thereof.

28. The method according to claim 23, wherein the gram-positive bacterium is a bacterium belonging to the phylum Actinobacteria or Firmicutes.

29. The method according to claim 23, wherein the gram-positive bacterium is a bacterium belonging to the phylum Actinobacteria.

30. The method according to claim 23, wherein the gram-positive bacterium is a coryneform bacterium, a bacterium belonging to the family Bifidobacteriumceae, a bacterium belonging to the family Dermabacteraceae, a bacterium belonging to the family Bacillaceae, a bacterium belonging to the family Enterococcaceae, or a bacterium belonging to the family Lactobacilluseae.

31. The method according to claim 23, wherein the gram-positive bacterium is a bacterium of the genus Corynebacterium, a bacterium of the genus Brevibacterium, a bacterium of the genus Bifidobacterium, a bacterium of the genus Brachybacterium, a bacterium of the genus Bacillus, a bacterium of the genus Enterococcus, or a bacterium of the genus Lactobacillus.

32. The method according to claim 23, wherein the gram-positive bacterium is Corynebacterium casei, Corynebacterium flavescens, Corynebacterium ammoniagenes (Corynebacterium stationis), Corynebacterium glutamicum, Brevibacterium casei, Bifidobacterium longum, Brachybacterium alimentarium, Bacillus subtilis, Enterococcus faecalis, Lactobacillus mali, Lactobacillus hilgardii, or Lactobacillus brevis.

33. The method according to claim 23, wherein the component (A) is added such that a concentration thereof at the time of eating or drinking is from 0.005 to 2% (w/w) in terms of dry weight of original cells of the gram-positive bacterium.

34. The method according to claim 23, further comprising a step of adding the following component (B) to the ingredient of the food product:
(B) one or more components selected from the group consisting of an L-amino acid, a nucleic acid and an organic acid.

35. The method according to claim 34, wherein at least the L-amino acid is added, and wherein the L-amino acid is L-glutamic acid.

36. The method according to claim 35, wherein the L-glutamic acid is added such that a concentration thereof at the time of eating or drinking is from 0.01 to 2% (w/w).

37. The method according to claim 34, which is carried out such that a content of the L-glutamic acid in the food product is from 0.1 to 20 parts by weight of the component (A) in terms of dry weight of original cells of the gram-positive bacterium.

38. The method according to claim 23, wherein the food product is a food product containing a spice.

39. The method according to claim 38, wherein a content of the spice in the food product is from 0.01 to 2% (w/w) as a concentration thereof at the time of eating or drinking.

40. The method according to claim 38, which is carried out such that the content of the spice in the food product is from 0.2 to 500 parts by weight of the component (A) in terms of dry weight of original cells of the gram-positive bacterium.

41. The method according to claim 38, wherein the spice is one or more spices selected from the group consisting of a spice of the family Lauraceae, a spice of the family Piperaceae, a spice of the family Lamiaceae, a spice of the family Apiaceae, a spice of the family Solanaceae, a spice of the family Myristicaceae, a spice of the family Alliaceae, a spice of the family Myrtaceae, a spice of the family Schisandraceae, a spice of the family Fabaceae, a spice of the family Polygonaceae, a spice of the family Brassicaceae, a spice of the family Zingiberaceae, and a spice of the family Rutaceae.

42. The method according to claim 38, wherein the spice is a pungent spice.

43. The method according to claim 23, wherein the gram-positive bacterium is a bacterium having an L-glutamic acid-producing ability and has one or more mutations selected from the mutations listed in Table 1
TABLE 1

| | | Pre- | Post- |
|---|---|---|---|
| | Genomic | mutation | mutation |
| No. | position | base | base |
| A-1 | 78,486 | C | T |
| A-2 | 83,592 | G | A |
| A-3 | 87,955 | C | T |
| A-4 | 90,041 | C | T |
| A-5 | 186,221 | C | T |
| A-6 | 193,010 | C | T |
| A-7 | 196,531 | C | T |
| A-8 | 225,429 | C | T |
| A-9 | 297,920 | G | A |
| A-10 | 320,354 | C | T |
| A-11 | 335,878 | C | T |
| A-12 | 341,763 | C | T |
| A-13 | 346,969 | C | T |
| A-14 | 349,856 | C | T |
| A-15 | 356,232 | C | T |
| A-16 | 357,008 | C | T |
| A-17 | 366,674 | G | A |
| A-18 | 369,871 | G | A |
| A-19 | 377,420 | G | A |
| A-20 | 378,652 | G | A |
| A-21 | 432,252 | C | A |
| A-22 | 439,021 | G | A |
| A-23 | 440,764 | G | A |
| A-24 | 454,682 | G | A |
| A-25 | 458,729 | G | A |
| A-26 | 470,562 | G | A |
| A-27 | 471,288 | G | A |
| A-28 | 472,023 | G | A |
| A-29 | 504,885 | G | A |
| A-30 | 505,785 | G | A |
| A-31 | 514,371 | G | A |
| A-32 | 518,684 | G | A |
| A-33 | 521,126 | G | A |
| A-34 | 524,551 | G | A |
| A-35 | 660,841 | C | T |
| A-36 | 732,121 | C | T |
| A-37 | 787,055 | C | T |
| A-38 | 806,047 | C | T |
| A-39 | 872,482 | G | A |
| A-40 | 878,069 | C | T |
| A-41 | 903,037 | C | T |
| A-42 | 922,802 | C | T |
| A-43 | 948,145 | C | T |
| A-44 | 955,819 | C | T |
| A-45 | 968,915 | C | T |
| A-46 | 973,013 | C | T |
| A-47 | 974,797 | C | T |
| A-48 | 994,815 | C | T |
| A-49 | 1,000,498 | C | T |
| A-50 | 1,019,704 | C | T |
| A-51 | 1,049,052 | C | T |
| A-52 | 1,069,322 | C | T |
| A-53 | 1,070,554 | C | T |
| A-54 | 1,131,016 | C | T |
| A-55 | 1,138,639 | C | T |
| A-56 | 1,162,588 | C | T |
| A-57 | 1,193,273 | C | T |
| A-58 | 1,203,146 | C | T |
| A-59 | 1,222,633 | C | T |
| A-60 | 1,226,969 | G | A |
| A-61 | 1,264,895 | G | A |
| A-62 | 1,268,790 | G | A |
| A-63 | 1,279,676 | G | A |
| A-64 | 1,363,909 | T | C |
| A-65 | 1,387,476 | G | A |
| A-66 | 1,401,171 | G | A |
| A-67 | 1,416,228 | C | T |
| A-68 | 1,420,034 | C | T |
| A-69 | 1,447,494 | C | T |
| A-70 | 1,448,318 | C | T |
| A-71 | 1,448,776 | C | T |
| A-72 | 1,451,922 | C | T |
| A-73 | 1,466,961 | C | T |
| A-74 | 1,503,736 | C | T |
| A-75 | 1,504,207 | C | T |
| A-76 | 1,505,998 | C | T |
| A-77 | 1,507,027 | C | T |
| A-78 | 1,544,310 | C | T |
| A-79 | 1,554,973 | C | T |
| A-80 | 1,558,509 | C | T |
| A-81 | 1,562,459 | C | T |
| A-82 | 1,572,716 | C | T |
| A-83 | 1,594,314 | C | T |
| A-84 | 1,602,545 | C | T |
| A-85 | 1,659,808 | C | T |
| A-86 | 1,682,132 | C | T |
| A-87 | 1,689,863 | C | T |
| A-88 | 1,744,963 | C | T |
| A-89 | 1,784,642 | C | T |
| A-90 | 1,814,866 | C | T |
| A-91 | 1,829,145 | C | T |
| A-92 | 1,852,511 | G | A |
| A-93 | 1,861,170 | G | A |
| A-94 | 1,902,133 | G | A |
| A-95 | 1,916,048 | C | T |
| A-96 | 1,917,434 | C | T |
| A-97 | 1,938,271 | C | T |
| A-98 | 1,949,357 | G | T |
| A-99 | 1,954,368 | C | T |
| A-100 | 1,967,997 | C | T |
| A-101 | 1,975,599 | C | T |
| A-102 | 2,141,466 | C | T |
| A-103 | 2,308,064 | C | T |
| A-104 | 2,310,428 | C | T |
| A-105 | 2,354,420 | C | T |
| A-106 | 2,449,270 | T | C |
| A-107 | 2,449,278 | C | A |
| A-108 | 2,449,291 | G | C |
| A-109 | 2,449,318 | G | A |
| A-110 | 2,496,945 | C | T |
| A-111 | 2,505,022 | C | T |
| A-112 | 2,505,285 | C | T |
| A-113 | 2,525,513 | G | A |
| A-114 | 2,565,856 | C | T |
| A-115 | 2,601,306 | G | A |
| A-116 | 2,615,688 | G | A |
| A-117 | 2,650,740 | G | A |
| A-118 | 2,653,259 | G | A |
| A-119 | 2,663,827 | G | A |
| A-120 | 2,667,322 | G | A |
| A-121 | 2,674,077 | G | A |
| A-122 | 2,679,915 | G | A |
| A-123 | 2,686,979 | G | A |
| A-124 | 2,693,950 | C | T |
| A-125 | 2,696,737 | C | T |
| A-126 | 2,706,442 | C | T |
| A-127 | 2,709,469 | C | T |
| A-128 | 2,711,214 | C | T |
| A-129 | 2,714,651 | C | T |
| A-130 | 2,721,339 | G | A |
| A-131 | 2,731,030 | G | A |
| A-132 | 2,746,202 | G | A |
| A-133 | 2,805,389 | C | T |
| A-134 | 2,816,733 | G | A |
| A-135 | 2,827,114 | G | A |
| B-1 | 29,724 | G | A |
| B-2 | 92,869 | G | A |
| B-3 | 116,733 | G | A |
| B-4 | 131,184 | G | A |
| B-5 | 156,247 | G | A |
| B-6 | 177,083 | G | A |
| B-7 | 184,379 | G | A |
| B-8 | 212,586 | G | A |
| B-9 | 282,162 | G | A |
| B-10 | 309,483 | G | A |
| B-11 | 376,164 | C | T |
| B-12 | 440,885 | C | T |
| B-13 | 479,120 | G | A |
| B-14 | 722,430 | G | A |
| B-15 | 745,504 | G | A |
| B-16 | 809,993 | G | A |
| B-17 | 859,643 | G | A |
| B-18 | 923,209 | G | A |
| B-19 | 924,973 | G | A |
| B-20 | 998,893 | C | T |
| B-21 | 1,062,144 | C | T |
| B-22 | 1,095,062 | C | T |
| B-23 | 1,102,484 | C | T |
| B-24 | 1,103,812 | C | T |
| B-25 | 1,105,749 | C | T |
| B-26 | 1,107,561 | C | T |
| B-27 | 1,205,722 | C | T |
| B-28 | 1,233,449 | C | T |
| B-29 | 1,242,484 | C | T |
| B-30 | 1,248,388 | C | T |
| B-31 | 1,249,270 | C | T |
| B-32 | 1,291,377 | C | T |
| B-33 | 1,308,597 | C | T |
| B-34 | 1,329,535 | C | T |
| B-35 | 1,367,486 | C | T |
| B-36 | 1,382,065 | C | T |
| B-37 | 1,403,043 | C | T |
| B-38 | 1,433,914 | C | T |
| B-39 | 1,442,447 | C | T |
| B-40 | 1,501,903 | G | A |
| B-41 | 1,504,744 | C | T |
| B-42 | 1,651,403 | G | A |
| B-43 | 1,695,473 | G | A |
| B-44 | 1,779,939 | G | A |
| B-45 | 1,797,452 | G | A |
| B-46 | 1,801,284 | G | A |
| B-47 | 1,816,679 | G | A |
| B-48 | 1,832,252 | G | A |
| B-49 | 1,843,841 | G | A |
| 8-50 | 1,868,285 | G | A |
| B-51 | 1,879,922 | G | A |
| B-52 | 1,892,007 | G | A |
| B-53 | 1,916,016 | G | A |
| B-54 | 1,937,604 | G | A |
| B-55 | 1,947,044 | G | A |
| B-56 | 1,948,411 | G | A |
| B-57 | 1,948,649 | G | A |
| B-58 | 1,967,697 | G | A |
| B-59 | 1,974,137 | G | A |
| B-60 | 2,028,284 | C | T |
| B-61 | 2,050,998 | G | A |
| B-62 | 2,052,708 | G | A |
| B-63 | 2,054,372 | G | A |
| B-64 | 2,065,568 | G | A |
| B-65 | 2,067,167 | G | A |
| B-66 | 2,082,577 | G | A |
| B-67 | 2,121,006 | A | G |
| B-68 | 2,149,369 | C | T |
| B-69 | 2,159,680 | G | A |
| B-70 | 2,380,965 | G | A |
| B-71 | 2,477,728 | G | A |
| B-72 | 2,542,800 | G | A |
| B-73 | 2,570,107 | G | A |
| B-74 | 2,647,383 | G | A |
| B-75 | 2,726,248 | C | T |
| B-76 | 2,825,055 | C | T |
| B-77 | 2,837,078 | C | T |
| B-78 | 2,865,322 | C | T |
| B-79 | 2,872,907 | C | T |
| B-80 | 2,880,351 | C | T |
| B-81 | 2,889,394 | C | T |
| B-82 | 2,906,471 | C | T |
| B-83 | 2,927,044 | C | T |
| B-84 | 2,929,963 | C | T |
| B-85 | 2,940,673 | C | T |
| B-86 | 2,946,285 | C | T |
| B-87 | 2,962,909 | C | T |
| B-88 | 2,975,742 | C | T |
| B-89 | 2,987,052 | C | T |
| B-90 | 3,079,560 | C | T |
| B-91 | 3,083,927 | C | T |
| B-92 | 3,090,163 | C | T |

44. The method according to claim 23, further comprising a step of adding a culture of a gram-positive bacterium to the ingredient of the food product.
